(19) United States
(12) Patent Application Publication
(10) Pub. No.: US 2026/0179397 A1
(43) Pub. Date: Jun. 25, 2026

(54) SYSTEM FOR HIGH-THROUGHPUT DATA MEASUREMENTS OF SINGLE SYNAPSES

(71) Applicant: UNIVERSITY OF MARYLAND, BALTIMORE, Baltimore, MD (US)

(72) Inventors: Samuel T. BARLOW, Baltimore, MD (US); Thomas A. BLANPIED, Baltimore, MD (US)

(21) Appl. No.: 19/429,862

(22) Filed: Dec. 22, 2025

Related U.S. Application Data
(60) Provisional application No. 63/736,957, filed on Dec. 20, 2024.

(51) Int. Cl. G06V 20/69; G02B 21/00; G02B 21/36; G06T 7/00; G06V 10/25; G16H 30/20

(52) U.S. Cl. CPC G06V 20/695; G02B 21/0076; G02B 21/365; G06T 7/0012; G06V 10/25; G06V 20/693; G16H 30/20; G06T 2207/10056; G06T 2207/30024

(57) ABSTRACT

Techniques for measuring single synaptic signals under varying experimental conditions include controlling a video recording microscope to capture, at multiple different times, a first imaged area in a sample holder as a video frame when the sample holder is disposed on a stage and holds a sample of neuronal tissue combined with at least one fluorophore that emits a corresponding electromagnetic wavelength in a synapse during synaptic activity. At least one synaptic region of interest is determined based on a group of pixels in the first imaged area that record electromagnetic emissions from the fluorophore at the different times. An ordered time series of emission intensity is recorded in each of the synaptic region of interest. Peak emission intensity values in the time series are corrected for transmitter label transients. The ordered time series with corrected peak emission intensity values are stored in a data structure with a standard format.

### CROSS-REFERENCE TO RELATED APPLICATIONS

This application is a United States nonprovisional application which claims the benefit of U.S. provisional application Ser. No. 63/736,957, filed 20 Dec. 2024. The entire contents of the aforementioned application is hereby incorporated by reference as if fully set forth herein.

### GOVERNMENT FUNDING SUPPORT

This invention was made with government support under Grant nos. MH080046 and MH119826, awarded by the National Institutes of Health. The government has certain rights in the invention.

### BACKGROUND OF THE INVENTION

### 1. Field of the Invention

This invention relates to the general field of neuroscience and more specifically to optical systems and methods to measure the functional heterogeneity of single neuronal synapses.

### 2. Background of the Invention

Synapses are the fundamental information processing modules in the brain, performing computations that dictate how electrical activity propagates across neural circuits. Thus, a major goal for neuroscience is to identify the basic functional properties of individual synapses which define their computational output, such as vesicle release probability (Pᵣ), the magnitude and variance of receptor activation, and short-term plasticity behavior. However, the enormous diversity that exists among synapses is a significant barrier to achieving a quantitative understanding of synaptic function. The distinct transcriptomic identities of pre- and postsynaptic neurons drive expansive proteomic diversity among synapses, and synapses are also plastic, with further speciation emanating from each synapse's unique history of activity. Synapse functional diversity is reflective of this deep proteomic diversity, with Pᵣ varying widely between synapse types (0.05 to 1). Pᵣ can fluctuate across stimulus frequencies as new vesicle populations or short-term plasticity mechanisms are engaged, properties which also exhibit a dependence on synapse identity. How these permutations of presynaptic properties impact the activation probability of NMDA receptors (NMDARs) will define which patterns of activity lead to synapse strengthening or weakening, constituting another axis of synaptic heterogeneity with implications for neural circuit development. NMDARs (N-methyl-D-aspartate receptors) in synapses are crucial glutamate receptors acting as coincidence detectors, requiring both glutamate binding and postsynaptic depolarization (Mg²⁺ block removal) to open, allowing Ca²⁺ influx for synaptic plasticity (learning/memory), synapse formation, and maturation, with distinct functions for synaptic vs. extrasynaptic receptors affecting cell survival or death pathways. They're key to learning by strengthening or weakening connections between neurons.

Due to their expansive diversity, a quantitative understanding of synaptic communication across single neurons and circuits will only be achieved through a synapse-by-synapse readout of synaptic function. Optical methods are well-positioned to meet this need, and direct measurement of synaptic functional properties has been demonstrated using a variety of fluorescent indicators.

### SUMMARY OF THE INVENTION

Several barriers must be overcome to leverage these tools at scale. First, detailed dissection of synaptic function requires a variety of stimulation protocols, chemical conditions, and imaging modalities, resulting in complex experimental paradigms. To acquire these data efficiently and reproducibly, it is desirable to fully automate microscopy, electrical stimulators, and fluidics. Second, to process large datasets, automated segmentation methods that can extract and analyze the same synapses across hundreds of video recordings are essential. Third, intensity-time recordings from individual synapses must be baseline-corrected and normalized to ΔF/F before fluorescence signals can be extracted and analyzed. Indeed, major software packages have been developed to accelerate segmentation and fluorescence signal extraction for calcium (e.g., CaImAn, FIOLA) and voltage imaging (e.g., VoIPy) in vivo, but there are no comprehensive software packages for analysis of synaptic function. Finally, fluorescence data must be converted to interpretable statistics for insight into synaptic functional properties.

Thus, current technology faces several significant technical shortcomings in analyzing synaptic function. For example, the data source is a major limitation. Existing technologies are unable to analyze synaptic-level data for large numbers of synapses because the data is typically sourced from cellular-level sensors. Consequently, synaptic data is averaged across many synapses, resulting in a loss of detailed, synapse-specific information. As a further example, the volume of data generated by traditional methods is substantial, making it challenging to process and interpret the information quickly. This large data volume can overwhelm existing processing capabilities, leading to delays and inefficiencies. In addition, data usability is a critical issue. Current technologies often produce standardized, inflexible outputs that do not allow for detailed customization or manipulation by the end-user. This lack of flexibility can be a significant limitation when specific, nuanced analyses are required, such as in the study of synaptic function. These shortcomings highlight the need for advanced technologies that can provide detailed, synaptic-level data, handle large volumes of data efficiently, and offer customizable outputs for precise analysis.

In summary, traditional methods for analyzing synapse function generate large volumes of data, making it difficult to process and interpret the information in a timely manner. This is particularly challenging when studying synapse dysfunction in various diseases, where rapid and accurate analysis is crucial for understanding disease mechanisms and developing therapeutic interventions. Thus, there is a need for improvements to high-throughput analysis of single synapse function, including a need to image and analyze the activity of many synapses quickly and efficiently.

The invention described herein thus provides embodiments related to optical systems and methods to measure the functional heterogeneity of single neuronal synapses. Synapses are highly heterogeneous. The systems and methods are configured to include a variety of sensors and fluorescent reporters. For example, one embodiment of the invention includes the third-generation intensity-based glutamate sensing fluorescent reporter (iGluSnFR3), which allows robust detection of glutamate release from single presynapses to provide quantitative access to basic functional properties such as basal release probability and short-term plasticity dynamics.

Existing software packages possess solutions for drift correction, segmentation, and intensity-time trace analysis, but none have been specialized for analysis of iGluSnFR3 recordings, and large segments of the code base were either unnecessary or challenging to customize.

Further provided is a modular approach to allow end-to-end, high-throughput collection and analysis of hundreds of synaptic recordings, such as iGluSnFR3 recordings, through a combination of hardware automation, batch segmentation, and automatic analysis of iGluSnFR3 fluorescence transients. Thus, the scalable, versatile approach enabled deep functional profiling of presynaptic functional heterogeneity (e.g. number of quanta released, Pᵣ, paired-pulse ratio, Readily Releasable Pool (RRP) size) across hundreds of boutons, which allows separation of boutons into functional classes according to their iGluSnFR3 responses. RRP size refers to the number of synaptic vesicles immediately available for release at a synapse, varying greatly by bouton type and location, from around 4-10 vesicles in small hippocampal excitatory synapses to potentially over 100 in larger or tonic terminals, influencing spontaneous release rates and overall neurotransmission. This pool's size isn't fixed, changing with activity, but serves as a key factor in determining how much neurotransmitter is released per action potential. Still further, the system can be extended across the synaptic cleft by combining iGluSnFR3 with a red-shifted, postsynaptically-targeted Ca⁺⁺ reporter to simultaneously image the ionotropic activation of postsynaptic NMDARs by endogenous glutamate release at single dendritic spines.

Directly imaging the flow of information during synaptic transmission at single dendritic spines enables detailed interrogation of synaptic functional heterogeneity and the patterns of glutamatergic activity which favor NMDAR-mediated plasticity induction. The systems and methods can discriminate populations of synapses that respond to pharmacological manipulation from those which are non-responsive. Thus, this invention provides (in multiple embodiments) systems and methods for synapse-by-synapse structure-function analyses to untangle synapse heterogeneity across single neurons and circuits.

In a first set of embodiments, a system for measuring single synaptic signals under varying experimental conditions includes a video recording microscope, at least one computer memory, and at least one processor. The video recording microscope is configured to view, and capture as a video frame, an imaged area in a sample holder disposed on a stage. The sample holder is configured to contain a sample of neuronal tissue combined with at least one fluorophore that emits a corresponding electromagnetic wavelength in a synapse during synaptic activity. The at least one memory includes one or more sequences of instructions, wherein the at least one memory and the one or more sequences of instructions are configured to, with the at least one processor, cause the processor to perform at least the following steps. A step is included to control the video recording microscope to capture, at a plurality of different times, a first imaged area in the sample holder when the sample holder is disposed on the stage and holds the sample of neuronal tissue combined with the fluorophore. A step is included to determine at least one synaptic region of interest based on a group of a plurality of pixels in the first imaged area that record electromagnetic emissions from the at least one fluorophore at the plurality of different times. A step is included to record an ordered time series of emission intensity in each of the at least one synaptic region of interest at the plurality of times. A step is included to correct peak emission intensity values in the time series for known fluorophore transients. A step is included to store the ordered time series with corrected peak emission intensity values in a data structure with a standard format.

In some embodiments of this set, the at least one memory and the one or more sequences of instructions are further configured to cause the processor to perform the following steps before said step to correct peak emission intensity values in the time series for transmitter label transients. A step is included to remove a first set of outlier emission values from the ordered time series based on a rolling median value to produce a first baseline time series in each of the at least one synaptic region of interest. Another step is included to record a rolling average time series of the first baseline time series in each of the at least one synaptic region of interest. Still another step is included to use the rolling average to determine a corrected normalized ordered time series of emission intensity and any potential fluorescence signal in each of the at least one synaptic region of interest. Further, a step is include to record a new baseline ordered time series of emission intensity based on a rolling average excluding outliers and potential signals. Still further, a step is included to use new baseline ordered time series of emission intensity to record a new normalized time ordered time series for each synapse for each fluorophore of the at least one fluorophore in each of the at least one synaptic region of interest.

In some embodiments of the first set, the stage is moveable; and the at least one memory and the one or more sequences of instructions are further configured to cause the processor to cause the moveable stage to move such that the video recording microscope views, and capture as a video frame, a second different imaged area in the sample holder.

In some embodiments of the first set, the video recording microscope is a confocal microscope configured to bring into focus a selectable depth in the sample held in the sample holder on the stage; and the at least one region of interest is a three dimensional region of interest comprising multiple depths in the sample.

In some embodiments of the first set, the system includes an electrical stimulation apparatus configured to apply an electrical voltage across a sample in the sample holder on the stage. The at least one memory and the one or more sequences of instructions are configured to, with the at least one processor, cause the processor to operate the electrical stimulation apparatus.

In some embodiments of the first set the system includes a perfusion apparatus configured to introduce at least one fluid into the sample holder on the stage. The at least one memory and the one or more sequences of instructions are further configured to, with the at least one processor, cause the processor to operate the perfusion apparatus. In some of these embodiments, the fluid comprises a solution of calcium ions.

In some embodiments of the first set, a first fluorophore of the at least one fluorophore is configured to emit a corresponding electromagnetic wavelength upon contact with glutamate. In some of these embodiments, the first fluorophore is iGluSnFR3.

In some embodiments of the first set, a first fluorophore of the at least one fluorophore is bound to a protein in a wall of a vessicle that holds a neurotransmitter. In some of these embodiments, the first fluorophore is synaptophysin-mRuby.

In some embodiments of the first set, wherein a first fluorophore of the at least one fluorophore is iGluSnFR3 and a second fluorophore of the at least one fluorophore is synaptophysin-mRuby.

In other sets of embodiments, a method and a non-transient computer-readable medium are configured to perform the steps of the above system.

### BRIEF DESCRIPTION OF THE DRAWINGS

Certain embodiments are illustrated by way of example, and not by way of limitation in the figures of the accompanying drawings.

FIG. 1A is a block diagram showing an end-to-end system for the automated imaging approach, according to an embodiment.

FIG. 1B is a block diagram that illustrates an example of imaged areas in a sample dish, according to an embodiment.

FIG. 2A through FIG. 2C are images that depict examples of time-lapse recordings along axons that can be batch segmented based on a fluorescent marker iGluSnFR3 or Synaptophysin-mRuby to produce one form of synaptic regions of interest, according to an embodiment.

FIG. 2D shows the algorithm's output for two regions of interest. The left traces show stimulus-evoked iGluSnFR3 activity; the right traces show spontaneous, AP-independent iGluSnFR3 transients, according to an embodiment.

FIG. 2E shows the exponential decay time constant (τ_{decay}), full-width at half-maximum (t½), 10-90% rise time (trise), 90-10% decay time (t_{decay}), and for stimulus-evoked transients, the time interval between peak and stimulus onset (Δt), fit to the data according to an embodiment.

FIGS. 2F and 2G show an example of a time lapse peak activity in the imaged area used as a second form of synaptic region of interest based on activity, overlaid with a time lapse from a single fluorophore, according to an embodiment.

FIG. 3 is a flow chart that illustrates an example of a method for operating the system of FIG. 1A, according to an embodiment.

FIG. 4 is a flow chart showing more detail in several steps of the method of FIG. 3, according to an embodiment.

FIG. 5A shows an example of an imaging region (51.2×51.2 μm) with an axon co815 expressing iGluSnFR3 (grey LUT) and Synaptophysin-mRuby3 (fire LUT), according to an embodiment.

FIG. 5B shows a spatial map of batch segmented ROIs according to Synaptophysin-mRuby3 expression (blue) or detected iGluSnFR3 activity (red) at [Ca2+]bath=2 mM for one trial, according to an embodiment.

FIG. 6A shows data for the number of ROIs detected per imaging region, according to an embodiment.

FIG. 6B shows data for the average AP821 independent, spontaneous iGluSnFR3 transients at each [Ca⁺⁺] bath for either segmentation method, marker first then activity, according to an embodiment.

FIG. 7A shows a normalized histogram of peak ΔF/F collected via marker or activity segmentation across [Ca⁺⁺] bath, according to an embodiment.

FIG. 7B shows a normalized histogram of τ_{decay}, according to an embodiment.

FIG. 7C shows a normalized histogram of t_{1/2}, according to an embodiment.

FIG. 8 shows the averaged iGluSnFR3 response for stimulus-evoked and spontaneous glutamate release across [Ca⁺⁺]bath=0.5, 1, 2, or 4 mM, according to an embodiment.

FIG. 9 shows the scatterplot of average peak amplitude (peak ΔF/F) vs. [Ca²⁺] bath for stimulus evoked and spontaneous iGluSnFR3 transients, according to an embodiment.

FIG. 10A is a normalized histogram of peak ΔF/F comparing identified spontaneous (light) with stimulus-evoked (dark) iGluSnFR3 transients at each [Ca⁺⁺]_{bath}, according to an embodiment.

FIG. 10B is a normalized histogram of τ_{decay}, according to an embodiment.

FIG. 11 is a scatterplot of the ratio of evoked response (E_{ΔF/F}) to spontaneous response (S_{ΔF/F}), i.e., E_{ΔF/F}/S_{ΔF/F} vs. [Ca⁺⁺]_{bath}, according to an embodiment.

FIG. 12 a scatterplot of variance (σ2) of E_{ΔF/F}/S_{ΔF/F} vs. mean (\tilde{x}) of E_{ΔF/F}/S_{ΔF/F} in an example of a bouton, according to an embodiment.

FIG. 13 is a violin plot of the distribution of solved Nsites per bouton, according to an embodiment.

FIG. 14 is a scatterplot of E_{ΔF/F}/S_{ΔF/F}/Nsites vs. [Ca⁺⁺]_{bath}, according to an embodiment.

FIG. 15 is a graph showing data on the probability of measuring stimulus-evoked iGluSnFR3 transient (PiGlu), according to an embodiment.

FIG. 16A is a plot showing the coefficient of variation (CB) of the peak ΔF/F for measured iGluSnFR3 transients, according to an embodiment.

FIG. 16B and FIG. 16C are plots showing the CV of τ_{decay} and the CV of Δt, respectively, according to an embodiment.

FIG. 17 is a UMAP representation of statistics of iGluSnFR3 activity at 251 boutons, according to an embodiment.

FIG. 18A shows data for PiGlu vs. [Ca⁺⁺] bath, grouped by cluster ID, according to an embodiment.

FIG. 18B shows the estimated number of synaptic vesicles (SVs) released (E_{ΔF/F}/S_{ΔF/F}) vs. [Ca⁺⁺]_{bath}, according to an embodiment.

FIG. 18C shows the estimated RRP size (Nsites) vs. cluster ID, according to an embodiment.

FIG. 19 is an example imaging area 192 with each bouton separated by cluster ID, according to an embodiment.

FIG. 20 is a set of graphs showing the averaged iGluSnFR3 waveform of single action potential (AP) trials for bouton 34, according to an embodiment.

FIG. 21A presents the observed probability, PiGlu and the calculated uniform Pr vs. [Ca⁺⁺]_{bath}, according to an embodiment.

FIG. 21B presents the E_{ΔF/F}/S_{ΔF/F} curve for Bouton 34 vs. [Ca2+]_{bath}, according to an embodiment.

FIG. 21C presents a scatterplot of variance (62) of E_{ΔF/F}/S_{ΔF/F} vs. mean (\tilde{x}) E_{ΔF/F}/S_{ΔF/F} for Bouton 34, according to an embodiment.

FIG. 22 shows the averaged iGluSnFR3 response for each stimulus protocol administered in this experiment across [Ca⁺⁺]bath=1, 2 mM, according to an embodiment.

FIG. 23 shows the effects of subtraction for determining paired-pulse ratios (PPRs) at individual boutons, according to an embodiment.

FIG. 24 is a set of violin plots showing the value of PPR as a function of [Ca⁺⁺] bath, interstimulus interval (ISI), according to an embodiment.

FIG. 25 shows categorized boutons according to which ISI produced the maximum value of PPR for boutons, termed the “Facilitation Bias,” according to an embodiment

FIG. 26A, FIG. 26B, and FIG. 26C show measures of plasticity mapped to an example of an imaging area for [Ca⁺⁺]_{bath}=1 mM, according to an embodiment.

FIG. 27A shows individual trial responses by [Ca⁺⁺]_{bath} with the average response for Bouton 5, according to an embodiment.

FIG. 27B shows PPR curves across ISIs for 1 mM and 2 mM Ca⁺⁺ for Bouton 5.

FIG. 28A shows individual trial responses color-coded by [Ca⁺⁺]_{bath} with the averaged response for Bouton 9, according to an embodiment.

FIG. 28B shows PPR curves across ISIs for 1 mM and 2 mM Ca⁺⁺ for Bouton 9, according to an embodiment.

FIG. 29 is a set of photographs showing a example of an imaging region 192 with a neuron expressing both iGluSnFR3 (1^{st} column) and spine-HaloTag™ dyed with JF646-BAPTA-HTL-AM (2^{nd} column) and merged (3^{rd} column), according to an embodiment.

FIG. 30A presents a ΔF/F vs. time trace with spontaneous activity collected from iGluSnFR3 (green) and JF646-BAPTA-AM (magenta) from a single dendritic spine head, according to an embodiment.

FIG. 30B is a scatterplot of JF646 peak amplitude vs. the iGluSnFR3 peak amplitude for the spine in FIG. 30A, according to an embodiment.

FIG. 31 shows the averaged waveforms of all collected transmission events from spines before and after exposure to a control solution or a solution containing 100 μM AP5 iGluSnFR3 (top) and JF646-BAPTA (bottom), according to an embodiment.

FIG. 32 is a set of plots of population correlations between iGluSnFR3 and JF646-BAPTA signals from normalized transmission events by spine (z-score), according to an embodiment.

FIG. 33 shows an example of an imaging area with dendritic spine ROIs shaded according to the recorded number of transmission events (Nₜᵣₐₙₛₘᵢₛₛᵢₒₙ), according to an embodiment.

FIG. 34A and FIG. 34B show time-aligned iGluSnFR3 and JF646-BAPTA signals for each transmission event for Spine 4 and Spine 20, according to an embodiment.

FIG. 35 shows the distribution of τ_{decay} vs. spine identity, according to an embodiment.

FIG. 36 plots the average iGluSnFR3 response in response to stimulus across [Ca⁺⁺]_{bath} and cluster ID, according to an embodiment.

FIG. 37A through FIG. 37K are plots showing additional functional properties of the three bouton classes, according to an embodiment. FIG. 37A, coefficient of variation (CV) of peak ΔF/F vs. [Ca⁺⁺]_{bath}; FIG. 37B, CV of τ_{decay} vs. [Ca⁺⁺]_{bath}; FIG. 37C, CV of Δt vs. [Ca⁺⁺]_{bath};

FIG. 37D, τ_{decay} vs. [Ca⁺⁺]_{bath}; FIG. 37E, Δt vs. [Ca⁺⁺]_{bath}; FIG. 37F, t½ vs. [Ca⁺⁺]_{bath}; FIG. 37G, trise (10-90%) vs. [Ca⁺⁺]_{bath}; FIG. 37H, τ_{decay} (90-10%) vs. [Ca⁺⁺]_{bath}; FIG. 37I, uniform release probability, Pr vs. [Ca⁺⁺]_{bath}; FIG. 37J, RRP fraction released per AP (E_{ΔF/F}/S_{ΔF/F})/Nsites vs. [Ca⁺⁺]_{bath}; FIG. 37K, binomial model Q vs. cluster ID.

FIG. 38A and FIG. 38B show exemplary imaging regions color-coded according to the slope of their correlation between JF646 and iGluSnFR3 signals, before (FIG. 38A) and after (FIG. 38B) wash-in of the control solution, according to an embodiment.

FIG. 39A shows the correlated iGluSnFR3 (top) and JF646 (bottom) activity before and after wash-in of control solution, according to an embodiment.

FIG. 39B is a scatter plot of JF646 amplitude vs. iGluSnFR3 amplitude, before and after wash-in of control solution, according to an embodiment.

FIG. 40A and FIG. 40B show the same imaging region before and after wash-in of a solution containing 100 μM DL-AP5, an NMDAR antagonist, respectively, according to an embodiment.

FIG. 41A and FIG. 41B report on the activity at a spine indicated by the arrow shown in FIG. 40A through FIG. 40B. In FIG. 41A, the correlated iGluSnFR3 (top) and JF646 (bottom) activity before and after wash-in of 100 μM DL-AP5. FIG. 41B is a scatter plot of JF646 amplitude vs. iGluSnFR3 amplitude, before and after wash-in of 100 μM DL-AP5, according to an embodiment.

FIG. 42A and FIG. 42B show correlated iGluSnFR3 (top) and JF646 (bottom) activity before and after wash-in of 100 μM AP5, according to an embodiment.

FIG. 43 shows the imaging region from FIG. 19, shaded according to number of SVs released at 4 mM Ca⁺⁺, according to an embodiment.

FIG. 44A and FIG. 44B show the iGluSnFR3 responses at all [Ca⁺⁺]_{bath} tested, according to an embodiment.

FIG. 45 and FIG. 46 show divergent iGluSnFR3 behavior for PiGlu vs. [Ca⁺⁺]_{bath}, according to an embodiment.

FIG. 47 and FIG. 48 show divergent iGluSnFR3 behavior for estimated SVs released per AP (E_{ΔF/F}/S_{ΔF/F}) vs. [Ca2+]_{bath}, according to an embodiment.

FIG. 49 is a block diagram that illustrates a computer system 4900 upon which an embodiment of the invention may be implemented.

FIG. 50 illustrates a chip set 5000 upon which an embodiment of the invention may be implemented.

### DETAILED DESCRIPTION OF THE INVENTION

### 1. Overview

### 1.1 Structures

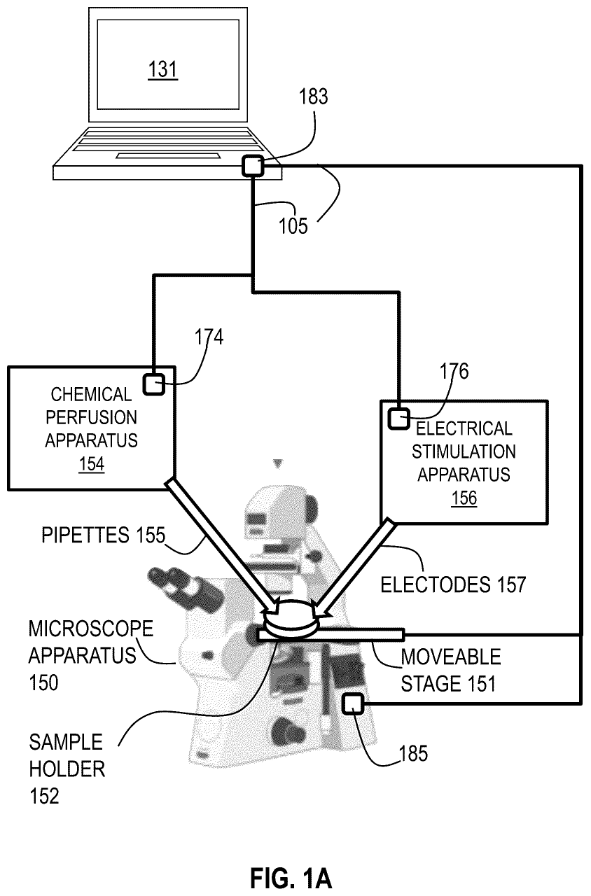

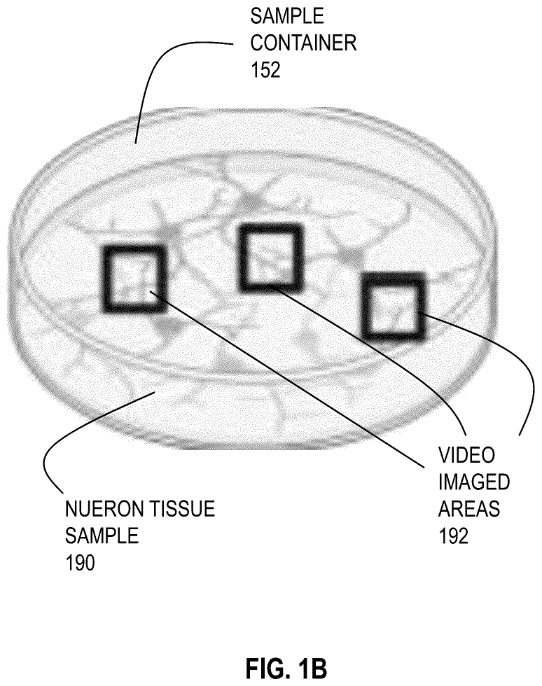

FIG. 1A is a block diagram showing an end-to-end system 100 for an automated imaging approach for measuring single synaptic signals under varying experimental conditions. The system includes a computer 131 serving as the central control unit with software module 183, which connects via wired or wireless connections 105 to peripheral devices, each with their own software modules. The software module includes one or more data structures to record protocols for perfusion and electrical stimulation and to record data captured by the system and data output by the system.

A module 174 controls a Chemical Perfusion Apparatus 154, which controls Pipettes 155 positioned above the moveable microscope stage and configured to dispense fluids, such as a solution of Calcium into a sample holder 152 disposed on the stage. A second module 176 controls an Electrical Stimulation Apparatus 156, which controls Electrodes 157 also positioned near the stage and configured to apply an electric voltage across a sample in the sample holder to stimulate neuronal tissue held in the sample holder.

A module 185 controls a Microscope Apparatus 150 with a Moveable Stage 151 mounted on it. The stage holds a Sample Holder 185 where neuronal tissue samples are placed for imaging. The pipettes and electrodes converge toward the sample holder area, indicating their positioning for delivering chemical solutions and electrical stimulation to the sample during experiments. Connection 105 links the microscope apparatus 150 to the computer system 131, enabling automated control of the stage movement and image capture.

The computer 131 includes at least one processor and at least one memory including one or more sequences of instructions. The at least one memory and the one or more sequences of instructions are configured to, with the at least one processor, cause the processor to control the video recording microscope, operate the electrical stimulation apparatus, and operate the perfusion apparatus. In some embodiments, the video recording microscope is a confocal microscope configured to bring into focus a selectable depth in the sample held in the sample holder on the stage.

FIG. 1B is a block diagram that illustrates an example of imaged areas in a sample dish. A sample container 152 is configured as a circular petri dish or similar vessel designed to hold a neuron tissue sample 190 for microscopic examination. Within the sample container, the neuron tissue sample is visible as a network of interconnected cellular structures with branching morphology characteristic of neuronal cells and their processes. Superimposed on the neuron tissue sample are three video imaged areas 192, represented as dark-bordered square regions positioned at different locations across the tissue sample.

These video imaged areas indicate discrete fields of view that can be captured by a video recording microscope during the imaging process. The arrangement of multiple imaged areas across the sample container illustrates the system's capability to capture video frames at different spatial locations within a single sample holder, enabling high-throughput data collection from multiple regions of the neuronal tissue. The moveable stage 151 allows the video recording microscope to sequentially view and capture different imaged areas within the sample holder, thereby facilitating the collection of synaptic activity data from numerous locations across the neuron tissue sample during experimental procedures. The moveable stage 151 also allows the sample holder 152 to be positioned under the pipettes 155 of the perfusion apparatus 154 and into the electric field of the electrodes 157 of the electrical stimulation apparatus 156.

### 1.2 Data

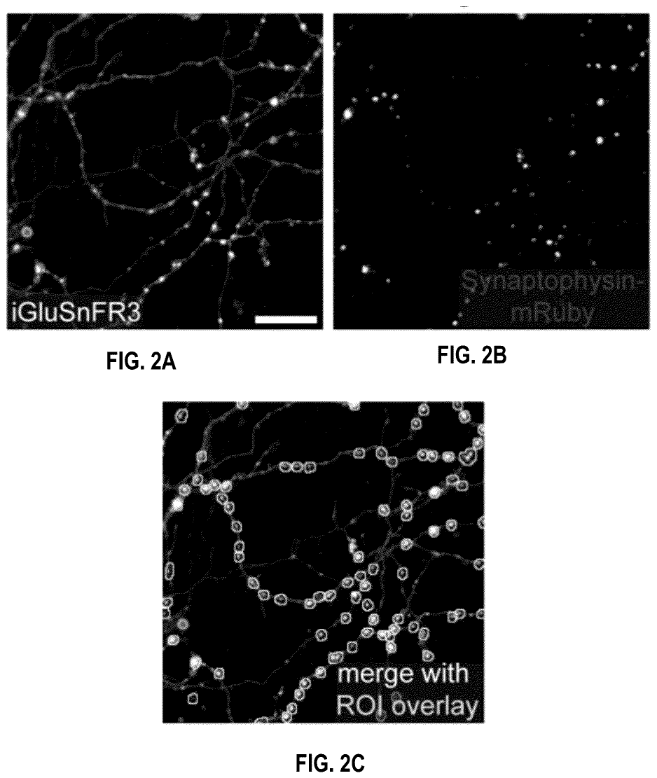

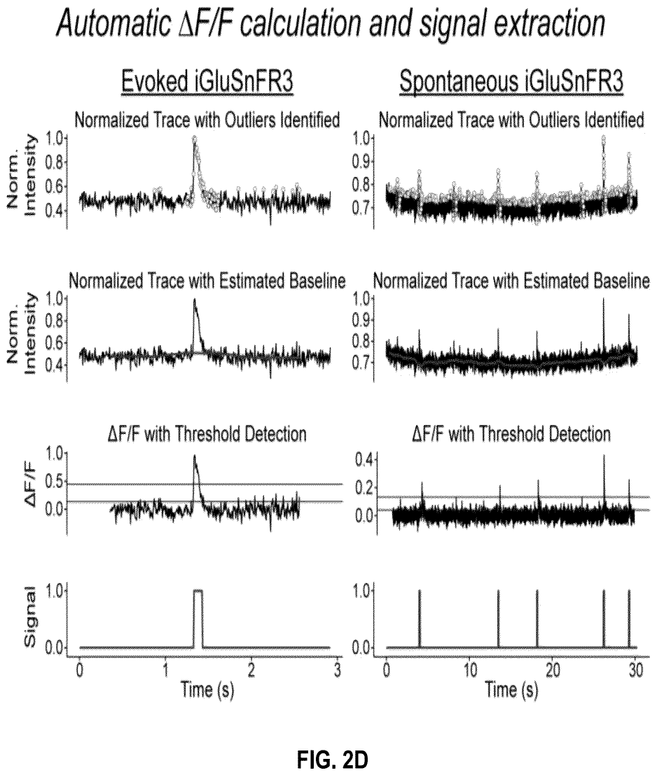

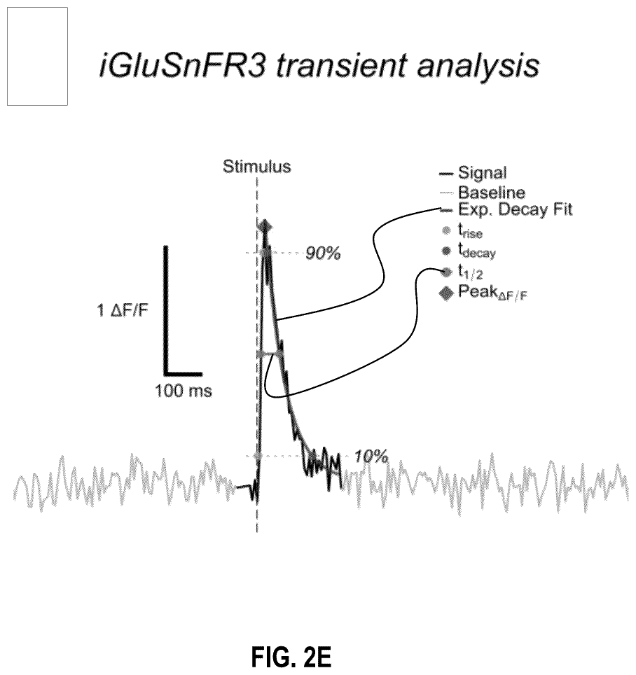

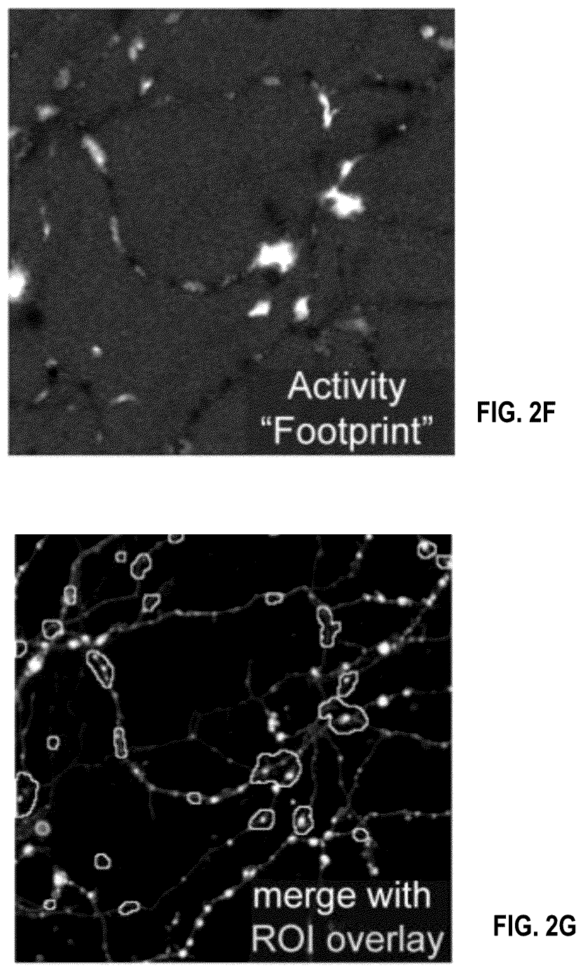

The sample of neuronal tissue is combined with at least one fluorophore that emits a corresponding electromagnetic wavelength in a synapse during synaptic activity. FIG. 2A through FIG. 2C depict examples of time-lapse recordings along axons that can be batch segmented based on fluorescent markers to produce synaptic regions of interest.

FIG. 2A shows neuronal tissue expressing the iGluSnFR3 fluorescent reporter, as an example of synaptic fluorophore. The iGluSnFR3 fluorophore is configured to emit a corresponding electromagnetic wavelength upon contact with glutamate, allowing robust detection of glutamate release from single presynapses. The image shows a network of neuronal processes, appearing as bright white linear structures against a dark black background, representing axons and dendrites extending across the field of view. Along these neuronal processes, numerous bright punctate structures are visible, which represent individual synaptic boutons or presynaptic terminals where glutamate release can be detected by the iGluSnFR3 sensor.

FIG. 2B displays the distribution of Synaptophysin-mRuby fluorescent markers, as an example of a second fluorophore associated with synapses within neuronal tissue. The image shows numerous bright punctate signals of varying intensities scattered across a dark background, with the fluorescent signals appearing in shades ranging from grey to bright white-yellow at their most intense points. These discrete fluorescent puncta represent individual synaptic vesicle clusters, as synaptophysin is a protein found in the membrane wall of synaptic vesicles that hold neurotransmitters. Thus, the synaptophysin-mRuby fluorophore is bound to a protein in a wall of a vesicle that holds a neurotransmitter.

FIG. 2C shows a merged image with region of interest (ROI) overlay, representing a composite view combining the raw fluorescence data with computationally determined region of interest boundaries. The image displays a network of neuronal processes with numerous bright spots representing sites of fluorescent activity. Outlined regions of interest have been overlaid on the image, delineating specific synaptic areas that have been automatically segmented for analysis.

FIG. 2D shows the algorithm's output for automatic ΔF/F calculation and signal extraction, comparing evoked and spontaneous synaptic activity in two parallel columns. The left column shows evoked iGluSnFR3 responses, while the right column displays spontaneous iGluSnFR3 transients.

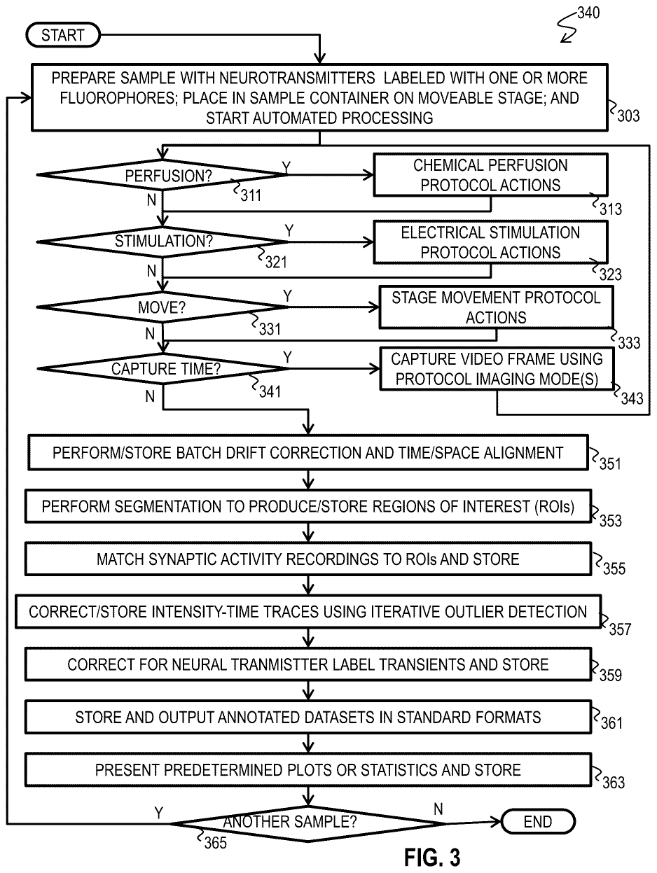

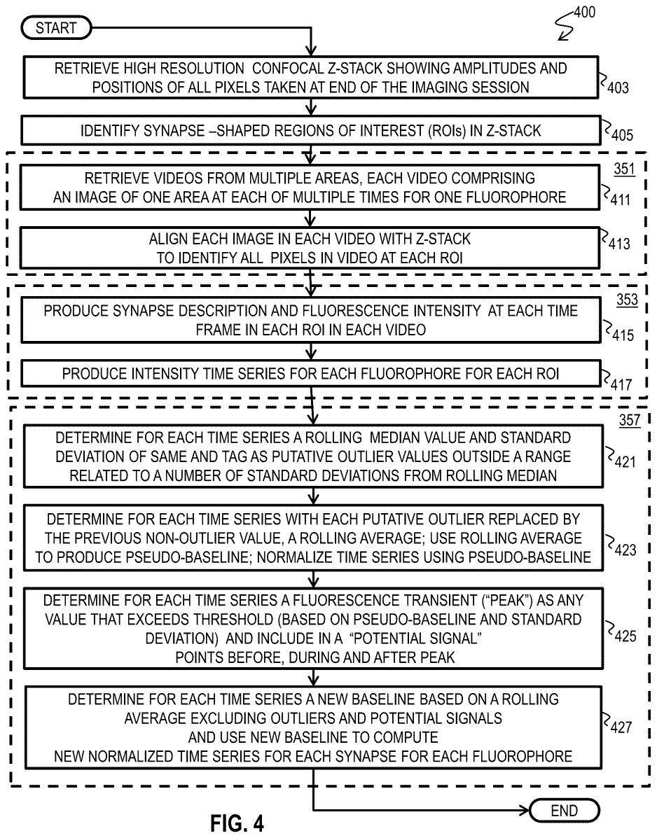

As described in more detail below with reference to the flow charts In FIG. 3 and FIG. 4, processing includes multiple stages: normalized traces with outliers identified, where raw fluorescence data appears with rolling median used for outlier detection and circles marking identified outlier points; normalized traces divided by baseline fluorescence (F) with estimated baseline depicted as curves overlaid on the fluorescence traces; ΔF/F traces, where ΔF is the difference between fluorescence after stimulation and the baseline fluorescence, with threshold detection where horizontal lines indicate detection thresholds; and final binary signal output with pulses indicating detected synaptic events at their corresponding time points. Baseline fluorescens is computed iteratively by first detecting outlier that could be noise and signals, then detecting signals then defining baseline absent outliers and signals, as described in more detail below.

FIG. 2E shows the transient analysis methodology for extracting quantitative parameters from fluorescence signals, especially moment of a stimulation event. In the illustrated example the transient response of iGluSnFR3 is used to fit the data from the corresponding channel of the image detection. The graph displays a characteristic synaptic fluorescence response with a sharp rise following a stimulus event, followed by an exponential decay. Several key measurement parameters are annotated: the exponential decay time constant (τ_{decay}), full-width at half-maximum (t_{1/2}), 10-90% rise time (tᵣᵢₛₑ), 90-10% decay time (t_{decay}), and for stimulus-evoked transients, the time interval between peak and stimulus onset (Δt).

FIG. 2F shows an activity footprint visualization from fluorescence microscopy imaging of neuronal tissue. The grayscale image shows a field of view containing multiple bright spots of varying sizes and intensities scattered across a darker background. These bright regions represent areas of synaptic activity where peak fluorophore emissions have been detected and recorded over time as describe in more detail below with reference to step 353 in FIG. 3.

This activity footprint serves as a second optional spatial map that enables automated segmentation algorithms to identify and isolate individual synapses for subsequent time series analysis and represents an example of an alternative definition of a region of interest (ROI). FIG. 2G shows a merged image with ROI overlay based on the activity footprint. Outlined regions of interest have been overlaid on the image, delineating specific synaptic areas identified from the activity footprint for analysis. This represents a second form of synaptic region of interest based on peak activity overlaid with time-lapse data from a single fluorophore These ROI can be compared to the more numerous smaller ROI depicted in FIG. 2C.

### 1.3 Methods

FIG. 3 is a flow chart that illustrates an example of a method 340 for operating the system of FIG. 1A.

The process begins with step 303, which involves preparing a sample with neurotransmitters labeled with one or more fluorophores, placing the sample in a sample container on a moveable stage, and starting automated processing. Any known method of marking synapses with fluorophores can be used, including glutamate detectors, labeled neurotransmitters, neurotransmitter vesicles labels, or Calcium detectors, introduced by perfusion or via genetic code introduced to the cell's protein formation process. In some embodiments, multiple fluorophores are introduced each with a corresponding unique wavelength for optical detection.

The flowchart then enters a decision loop structure containing three sequential decision diamonds. The first decision diamond 311 queries whether perfusion is required, and if yes, proceeds to step 313 for chemical perfusion protocol actions. The perfusion protocols are programmed into module 183 and driven by commands from module 183 for the application programming interface (API) of the perfusion module 174. The second decision diamond 321 queries whether stimulation is required, and if yes, proceeds to step 323 for electrical stimulation protocol actions. The electrical stimulation protocols are programmed into module 183 and driven by commands from module 183 for the application programming interface (API) of the electrical stimulation module 176. The third decision diamond 331 queries whether movement is required, and if yes, proceeds to step 333 for stage movement protocol actions, e.g., to move the sample container to the perfusion or electrical stimulation arms or to change the field of view 192 in the sample holder.

Following these conditional steps, decision diamond 341 queries whether it is capture time, and if yes, proceeds to step 343 to capture a video frame using protocol imaging modes. The modes can refer to colors as recorded by the mixture of three base colors in the color coded pixel intensities. The capture time can be after every interval in a range of 0.002 seconds (s) to 0.020 seconds (s), such as using sampling rates of 500 hertz (Hz) to 50 Hz.

The process then continues through a series of sequential processing steps.

Step 351 performs and stores batch drift correction and time/space alignment, as described in more detail below with reference to FIG. 4. Drift correction accounts for small stage movements between perfusion rounds at single imaging regions, and for distortion due to pressure from pipettes or electrodes. In various embodiments, step 351 demonstrates excellent spatial specificity for single boutons and allowing differentiation of boutons separated by less than 3 μm.

Step 353 performs segmentation to produce and store regions of interest (ROIs). The “ROI maps” were distributed to folders containing individual imaging trials. Step 355 matches synaptic activity recordings to ROIs and stores the data. If a fiducial marker of synapses is available, segmentation is performed using fiducial marker of synapses to generate a map of synapses. If fiducial marker of synapses is not available, segment using activity footprint to generate a map of synapses.

For fiducial marker of synapses, at the end of each iGluSnFR3 imaging session, confocal z-stacks were recorded of the same imaging fields to capture the position of Synaptophysin-mRuby puncta (representing putative boutons) along the axonal arbor. Most confocal z-stack image processing was performed with custom macros in FIJI according to known methods. Briefly, the middle 3.33 μm of each z-stack were extracted and converted to maximum intensity projections to eliminate putative boutons outside the widefield imaging plane. Background subtraction was performed by identifying the lowest 1% of pixel intensity per channel and subtracting this value from its respective image channel. A mask of the axonal arbor was constructed by automatically thresholding the iGluSnFR3 channel on the top 5% of pixel intensities. Binary axonal arbor masks were smoothed and small background particles were removed. Putative synaptic puncta were identified in the Synaptophysin-mRuby channel with the plugin SynQuant. Only Synaptophysin-mRuby ROIs within the boundary of the mask of the axonal arbor were analyzed. After drift correcting for small stage movements between perfusion rounds at single imaging regions, the “ROI maps” were distributed to folders containing individual imaging trials. A custom FIJI macro iterated through all subdirectories, opening videos and applying their respective ROI maps, populating a separate directory with .csv files of the intensity-time recordings at each ROI, which we then read into R for analysis.

For activity-based segmentation, a method described in Mendonca et al. 2022 was applied, with some modifications. Briefly, a moving average filter with a 5-point span was used to smooth the temporal profile of the iGluSnFR3 responses. A band-pass Gaussian filter (0.05-200 Hz) was then applied to amplify the iGluSnFR3 signal. At each pixel we subtracted the mean value and divided by the standard deviation, which had the effect of suppressing inactive stretches of the axonal arbor and amplifying active stretches (the “activity footprint” of the axon). From these, max intensity projections were created, which were thresholded on the top 3% of all pixels to generate ROI maps of activity along the axonal arbor. These “activity maps” were mapped to their respective videos and extracted .csv files of intensity-time recordings for each ROI, which were then read into R for analysis.

Step 357 corrects and stores intensity-time traces at each ROI using iterative outlier detection, described in more detail below in FIG. 4. Basically, to normalize iGluSnFR3 intensity-time traces to compute normalized fluorescent intensities across various captured images, ΔF/F, baseline fluctuations were corrected, and fluorescence signals extracted at scale, using a custom algorithm written in R (peakFinder.R). The algorithm proceeds in three stages, iteratively refining its approximation of the trace's baseline.

Step 359 corrects for neural transmitter label transients and stores the results. That is, the rise and exponential decay associated with a fluorophore is fit to the corrected normalized intensity-time traces to smooth the data and to identify the time(s) and numbers of any stimulation that induced the signal(s). In some embodiments, this step excludes a blanket of time points around the identified signals based on the expected duration of the signals, such as based on the known kinetics of the sensor and other characteristics. In some embodiments these points are excluded in step 425, described below, from calculating a rolling average of the signals. In at least one embodiment, the method iterates the excluding of points to progressively refine the signals. Thus, the method is configured to analyze signals and signal traces of synaptic function activities, including signals of relatively well-behaved traces and signals that include noise, and further wherein the signal includes shifts in a baseline, such as when a sensor moves around a cell.

Step 361 stores and outputs annotated datasets in standard formats, such as spreadsheet or comma separate value (CSV) text, enabling downstream analysis of synaptic functional properties such as basal release probability and short-term plasticity dynamics.

Step 363 presents predetermined plots or statistics and stores them. For example, step 363.

Finally, decision diamond 365 queries whether there is another sample to process, returning to the beginning of the loop if yes, or proceeding to END if no.

FIG. 4 is a flow chart showing more detail in several steps of the method of FIG. 3.

The method 400 begins at step 403, where the system retrieves a high resolution confocal Z-stack showing amplitudes and positions of all pixels at all depths in the sample, taken at the end of the imaging session. Step 405 involves identifying synapse-shaped regions of interest (ROIs) in the Z-stack.

Step 351, described above, encompasses steps 411 and 413, where step 411 retrieves videos from multiple areas, with each video comprising an image of one area at each of multiple times for one fluorophore, and step 413 aligns each image in each video with the Z-stack to identify all pixels in the video at each ROI.

Step 353, described above, contains steps 415 and 417, where step 415 produces a synapse description and fluorescence intensity at each time frame in each ROI in each video, and step 417 produces an intensity time series for each fluorophore for each ROI. Step 357 encompasses steps 421, 423, 425, and 427 related to baseline F correction and normalized signal detection ΔF/F.

Step 421 determines for each time series a rolling median value and standard deviation, tagging putative outlier values outside a range related to a number of standard deviations from the rolling median. The standard deviation is the standard deviation of the whole uncorrected time series. The rolling median is computed during a window of 400 to 800 ms, e.g., 400 to 750 ms, with successive windows one time step apart. In some embodiments, the threshold is 1.5 standard deviations from the median value. Thus, the algorithm flagged outlier indices as those which rise above a threshold of a (standard deviation) from the rolling median (0.75 s span) of the raw fluorescence intensity (median filter). Once a first approximation of the outliers is known, a more refined approximation of the baseline, F, was made on the raw intensity trace in step 423.

In step 423, a rolling median (0.75 s span) which excluded the known outlier indices was determined. The outlier indices were replaced with the last non-NA value (i.e. known intensity at non-signal indices) using a “last observation carried forward” function in R (na.locf( )).

In step 425, traces were adjusted to ΔF/F by dividing the raw intensity by the approximate baseline, also called a “pseudo baseline”.

In step 425, using a Schmitt trigger thresholding approach, putative fluorescence signals were identified when they exceeded an upper threshold of 3.5σ. The signals terminated when they decayed below a lower threshold of 1.5σ. The indices corresponding to these putative fluorescence signals were flagged. To identify the full extent of putative fluorescence signals and properly exclude them from the baseline approximation, additional points before and after putative fluorescence signals were flagged as signal indices. In some embodiments, the before and after points were selected according to the known rise and decay kinetics of the fluorescent sensor being imaged, as described above in step 359. Thus, in some embodiments, step 359 is a sub-step of step 425.

In step 427 With the original outliers and the putative signal indices flagged, a new baseline (F) was calculated from the rolling average of the raw intensity trace excluding outliers and putative signals, and the final iteration of ΔF/F was calculated. The final detection of fluorescence transients was achieved with a more stringent threshold (lower, 1.5σ, upper, 5σ) and annotated for further analysis.

In time series of JF646 fluorophore emissions, the slower kinetics of JF₆₄₆ and the frequent convolution of JF₆₄₆ transients with one another made it difficult to approximate the baseline and accurately convert traces to ΔF/F using a median filter. Therefore, a percentile filter approach was used in step 421 for these traces. In this case, the trace was divided into 10 equivalent time bins (e.g. 0-2 s, 2-4 s, etc.), and indices which comprised the bottom 30% of intensity values were identified in each bin. Similar to above, these were flagged as putative baseline indices, and the baseline fluorescence intensity at these indices were interpolated using na.locf( ). A rolling average of these points with a 0.75 second span was calculated to approximate the baseline. The trace was then adjusted to ΔF/F, and putative signals identified with a Schmitt trigger (lower, 1.5σ, upper, 3.5σ) in step 425. We then repeated this process with the putative signals being excluded from the percentile filter. The final iteration of ΔF/F was calculated and JF₆₄₆ transients were once again identified with a stringent threshold (lower, 1.5σ, upper, 5σ) and annotated for further analysis.

The ordered time series with corrected peak emission intensity values are stored in a data structure with a standard format, as described above in step 361.

These systems and techniques shortened the time lag between planning experiments to performing those experiments by operating experimental equipment automatically and generating publication-quality figures from weeks to days with fewer errors introduced by manual manipulations.

Provided according to several embodiments, are systems and methods for high-throughput analysis of single synapse function. At least one embodiment includes all-optical systems and methods to measure the functional heterogeneity of single synapses, segment synaptic activity (in terms of intensity and time), normalize synaptic activity with respect to the baseline using an iterative baseline identification algorithm. The systems and methods allow batch analysis of single synapse function of fluorescence recordings by correcting and normalizing fluorescence traces in a batch format and based on the known kinetics of the input fluorescent biosensor. In at least one embodiment, the system includes an iterative outlier detection to distinguish baseline noise from true signals. In another embodiment, the systems and methods outputs an annotated dataset (e.g., CSV file), such as for analysis in common software environments such as Excel, Origin, Python, MatLab, and R.

Thus this system provides a synaptic measurement pipeline that extracts presynaptic functional properties (e.g. release probability, frequency, and quantal content) of individual synapses using GluSnFR3 during electrical stimulation or action potential-independent paradigms. The pipeline extracts intensity-time traces from automatically segmented, putative synapses according to their GluSnFR3 activity. Because regions-of-interest display broad variation in noise levels and baseline stability, an iterative outlier detection approach was implemented which enables flexible identification of the baseline across a variety of trace conditions and improves the accuracy and precision with which we can determine GluSnFR3 ΔF/F. This high-throughput approach is configured to efficiently collect and analyze hundreds of optical recordings of stimulus-evoked or spontaneous glutamate release activity across the axonal arbors of cultured rat hippocampal neurons using the third-generation glutamate fluorescent reporter, iGluSnFR3. This embodiment leverages iGluSnFR3 to track the function of single, putative boutons in response to single stimulus.

For example, the system and method can be used in the development of therapeutics that act on synaptic activity, e.g., therapeutic development for addressing neurological and neuropsychiatric illnesses. In another embodiment, the system and method can be configured to test the efficacy of drugs targeting synapse function. In still another embodiment, the system and method can be configured in a personalized medicine method, such as for testing drugs on human-derived neurons. Thus, the system and method can be used in the development of targeted therapy.

### 2. Example Embodiments

This invention is not limited to the particular processes, compounds, compositions, or methods described in this section, as these may vary. The terminology used in the description is for the purpose of describing the particular versions or embodiments only, and is not intended to limit the scope of the present invention which will be limited only by the appended claims. Although any methods and materials similar or equivalent to those described herein can be used in the practice or testing of embodiments of the present invention, the preferred methods, devices, systems, compounds, compositions and materials are also described. Thus, the invention provides, according to multiple embodiments, and as described, non-limiting examples of embodiments of the systems and methods for synapse-by-synapse structure-function analyses to untangle synapse heterogeneity across single neurons and circuits

The present examples relate to all-optical methods to measure the functional heterogeneity of single synapses as applied to cultured rat hippocampal neurons. We focused on the third-generation intensity-based glutamate sensing fluorescent reporter (iGluSnFR3), which allows robust detection of glutamate release from single presynapses to provide quantitative access to basic functional properties such as basal release probability and short-term plasticity dynamics. Existing software packages possess solutions for drift correction, segmentation, and intensity-time trace analysis, but none were specialized for analysis of iGluSnFR3 recordings, and large segments of the code base were either unnecessary or challenging to customize.

We therefore developed a modular approach, enabling end-to-end, high-throughput collection and analysis of hundreds of iGluSnFR3 recordings, through a combination of hardware automation, batch segmentation, and automatic analysis of iGluSnFR3 fluorescence transients. This scalable, versatile approach enabled deep functional profiling of presynaptic functional heterogeneity (e.g. number of quanta released, Pr, paired-pulse ratio) across hundreds of boutons, which enabled separation of boutons into functional classes according to their iGluSnFR3 responses.

In addition, we extended the approach across the synaptic cleft by combining iGluSnFR3 with a red shifted, postsynaptically-targeted Ca⁺⁺ reporter to simultaneously image the ionotropic activation of postsynaptic NMDARs by endogenous glutamate release at single dendritic spines. Recall, NMDARs (N-methyl-D-aspartate receptors) in synapses are crucial glutamate receptors acting as coincidence detectors, requiring both glutamate binding and postsynaptic depolarization. Directly imaging the flow of information during synaptic transmission at single dendritic spines will enable detailed interrogation of synaptic functional heterogeneity and the patterns of glutamatergic activity which favor NMDAR-mediated plasticity induction. These innovations lay the groundwork for synapse-by-synapse structure-function analyses to untangle synapse heterogeneity across single neurons and circuits.

### 3. Embodiments of the Invention

### 3A. Introduction and Discussion

Here, we developed a high-throughput optical physiology approach to address several aspects of synaptic functional heterogeneity using iGluSnFR3 in hippocampal neuron cultures. First, we observed broad diversity in synaptic transmission strength across boutons, as both the estimated number of synaptic vesicles (SVs) released per stimulus and Readily Releasable Pool (RRP) (RRP) size exhibited broad inter-bouton variation. Second, by enabling systematic analysis of responses probed with complex stimulus paradigms and across multiple ionic or pharmacological conditions, we found that the basic characteristics (e.g. amplitude, variance) of stimulus-evoked iGluSnFR3 responses at single boutons were sufficient to separate boutons into multiple functional classes. Such functional classification of single synapses is useful for understanding synaptic heterogeneity across single neurons and circuits, and how synaptic function is perturbed by genetic and pharmacological manipulation. Third, diverse short-term plasticity behavior was observed: even boutons near one another and with similar basal release properties could exhibit a large range of paired-pulse facilitation dynamics, implying distinct configurations of the presynaptic release machinery.

The high-throughput approach to measure pre- and postsynaptic functional properties simultaneously, was leveraged to access NMDAR-mediated synaptic transmission at single spines by pairing iGluSnFR3 with a red-shifted Ca⁺⁺ sensor (JF₆₄₆-BAPTA). This approach allows future investigations to determine the patterns of glutamatergic activity which favor NMDAR activation across synapse types and how these may be disrupted in disease models which feature NMDAR dysfunction, opening new lines of inquiry into synaptic functional heterogeneity across single neurons and circuits.

Our approach combined three key technical innovations. First, we devised an automated hardware scheme controlled entirely by a Python script that supports the design of complex, versatile experiments which featured multiple imaging modes, stage locations, stimulus protocols, and perfusion rounds. Second, we developed a suite of easily customized scripts across ImageJ and MatLab for batch drift correction and segmentation of optical physiology recordings. These scripts are available in a preprint at domain org, subdomain doi at file 10.1101/2024.12.23.629904 posted Dec. 23, 2024. R-358 based file handling routines automatically matched recordings of synaptic activity to their appropriate maps of ROIs, allowing facile tracking the iGluSnFR3 activity at boutons across 40+ imaging rounds. Our custom R algorithm automatically corrected the baseline and normalized intensity-time traces, as described above, enabling the analysis of 10,000+ traces in tens of minutes on standard computers and automating the production of customizable outputs ranging from single-synapse functional reports to fully publication-quality summary figures. In addition, the algorithm extracted iGluSnFR3 transients according to the published kinetics of the fluorescent biosensor being imaged. In principle the algorithm can perform baseline correction, trace normalization, and feature extraction for a variety of fluorescent biosensors (e.g. GCaMP8f, JF₆₄₆-BAPTA). Custom R scripts were used to analyze these data and generate the figures presented herein. Together, these innovations constituted a data collection and analysis framework which shortened the time lag between experiments and publication-quality figures from weeks to days.

Synaptic strength is partly set by the number of SVs mobilized per AP, and understanding how this property varies across neurons and circuits is crucial to understanding how information flows in the brain. We used our high-throughput approach to directly estimate how many SVs were released per stimulus at single boutons by using quantal, AP-independent iGluSnFR3 transients as the benchmark for glutamate release from a single SV (E_{ΔF/F}/S_{ΔF/F}).

A fundamental parameter underlying synaptic strength is the number of synaptic vesicles (SVs) mobilized per action potential, (AP), e.g., due to electrical stimulation, and understanding how this property varies across neurons and circuits is crucial to understanding how information flows in the brain. The high-throughput approach was used to directly estimate how many SVs were released per stimulus at fields of single boutons by using quantal, AP-independent iGluSnFR3 transients as the benchmark for glutamate release from a single SV This is indicated by normalized fluorescence intensity evoked by a stimulus divided by the average normalized fluorescence intensity that arises spontaneously (E_{ΔF/F}/S_{ΔF/F}). As the probability of releasing a synaptic vesicle (Pᵣ) increased with increasing extracellular concentration of calcium ions ([Ca⁺⁺]_{bath}), boutons exhibited broad diversity in SVs released per stimulus, reflecting broad diversity in synapse strength. Since multiple Pᵣ states were sampled for each bouton, we were also able to perform quantal analysis for many of the boutons in our sample (n=122 boutons) and calculate their RRP size (Nₛᵢₜₑₛ). The average RRP size we measured for single boutons (Nₛᵢₜₑₛ=9.05±0.63) was in good agreement with ultrastructural measurements of docked SVs in a similar preparation (10.1±4.3, mean±s.d), Assuming the average active zone contains about 10 SVs in the RRP, our approach clearly captured boutons with multiple active zones, as RRP size ranged from 1-40 release sites. Thus, this unbiased, high-throughput approach likely captures presynapses that are normally omitted from morphological investigations and that may be regulated within the same bouton contacting one or more postsynaptic targets.

Given our access to both SVs released per stimulus and RRP size, the fraction of the RRP released per stimulus could be calculated for single boutons (see FIG. 14). FIG. 14 is a scatterplot of (EΔF/F/SΔF/F)/Nsites vs. [Ca⁺⁺] bath. RRP fraction trends were very similar at all boutons, suggesting that RRP fraction released per stimulus may be tightly regulated. Without wishing to be bound by theory, we expect that this technique could be used to measure RRP refilling and exhaustion during stimulus trains, another determinant of synaptic efficacy which may exhibit intersynapse variability.

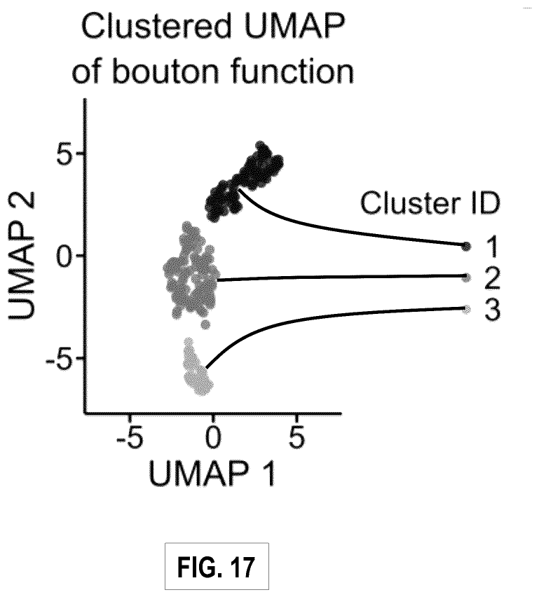

Having measured diverse SV release behavior and RRP sizes across boutons, the high-throughput approach captured substantial synaptic functional diversity. The basic characterization of stimulus-evoked iGluSnFR3 behavior was sufficient to separate boutons into multiple functional classes (see FIG. 17). FIG. 17 is a UMAP representation of statistics of iGluSnFR3 activity at 251 boutons. Twenty-four basic statistics describing each bouton's stimulus-evoked iGluSnFR3 responses were collected and projected in two dimensions via UMAP, which clustered into three discrete functional classes UMAP (Uniform Manifold Approximation and Projection) is a powerful non-linear dimensionality reduction tool in biology, widely used to visualize complex, high-dimensional data. Grouping the data by functional class, we observed that the key features for classification were the magnitude of the stimulated iGluSnFR3 response at 4 mM Ca⁺⁺ and whether boutons exhibited iGluSnFR3 activity at 0.5 mM Ca⁺⁺. Class 1 boutons exhibited reliable activity at 0.5 mM Ca⁺⁺ and also possessed the greatest number of release sites in their RRP (Nₛᵢₜₑₛ=14.7±2.0) In contrast, Class 3 boutons were never active at 0.5 mM Ca⁺⁺ and possessed small RRPs (Nₛᵢₜₑₛ=4.3±0.5).

One potential interpretation of these data is that each functional class represents boutons at a different developmental stage. Recent work at inhibitory basket cell-Purkinje cell synapses indicated that as these synapses matured, active zones increased in size and the coupling distance between release sites and Ca⁺⁺ channels decreased, increasing the reliability of synaptic transmission. Consistent with active zone expansion, RRP size expanded by ˜5 SVs per class from Class 3 to Class 1. iGluSnFR3 activity became much more probable from Class 3 to Class 1, consistent with reduced coupling distance between release sites and Ca⁺⁺ channels. This was most obvious at 1 mM Ca⁺⁺, where Class 3 boutons were reluctant to release glutamate (P_{iGlu}=0.19±0.02), while Class 1 boutons were almost always active (P_{iGlu}=0.89±0.02). Contrary to our expectations, however, the uniform Pᵣ derived from quantal analysis was nearly identical across classes for all Ca⁺⁺, suggesting that the distribution of release sites around Ca⁺⁺ channels did not differ between functional classes. However, we also note that iGluSnFR3 responses at Class 1 boutons had the fastest rise and decay kinetics of the three classes, which again supports a model in which Class 1 boutons are the most functionally mature of the observed bouton classes. But it remains unclear how the iGluSnFR3 response kinetics are affected by the alterations in the kinetics of the underlying SV exocytosis event.

An alternative explanation could be that Class 1 boutons are those which experience synaptic crosstalk from unlabeled boutons, but labeled boutons separated by only 1.8 μm possessed distinct iGluSnFR3 activity. By pairing our functional imaging approach with post-hoc immunocytochemistry or super-resolution imaging of presynaptic proteins, functional motifs of single boutons could be expected to be assigned to protein expression or protein organizational motifs to establish structure-function relationships for single synapses.

Short-term plasticity of the presynapse also plays a role in shaping synaptic computation. Depending on the input activity frequency, facilitation can occur, in which presynapses transiently increase Pᵣ to mobilize additional SVs upon stimulation. Given that the approach used here uncovered remarkable heterogeneity in basal glutamate properties, we probed whether heterogeneous short-term plasticity dynamics could be observed using paired stimuli. Nearly half of boutons (49%) exhibited facilitation which decayed as Interstimulus Interval (ISI) increased, exhibiting their largest PPR at ISI=60 milliseconds (ms). However, many boutons instead preferentially facilitated only when stimulated at longer ISIs (e.g. 100, 150 ms). These divergent facilitation properties might be explained simply by RRP size or functional class, in which certain RRP sizes or functional classes give rise to specific synaptic facilitation dynamics.

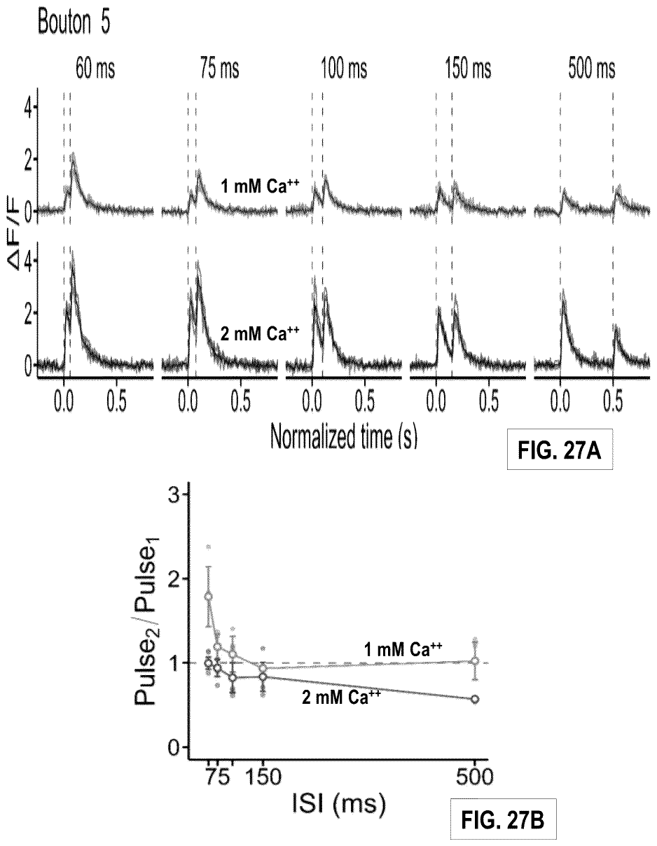

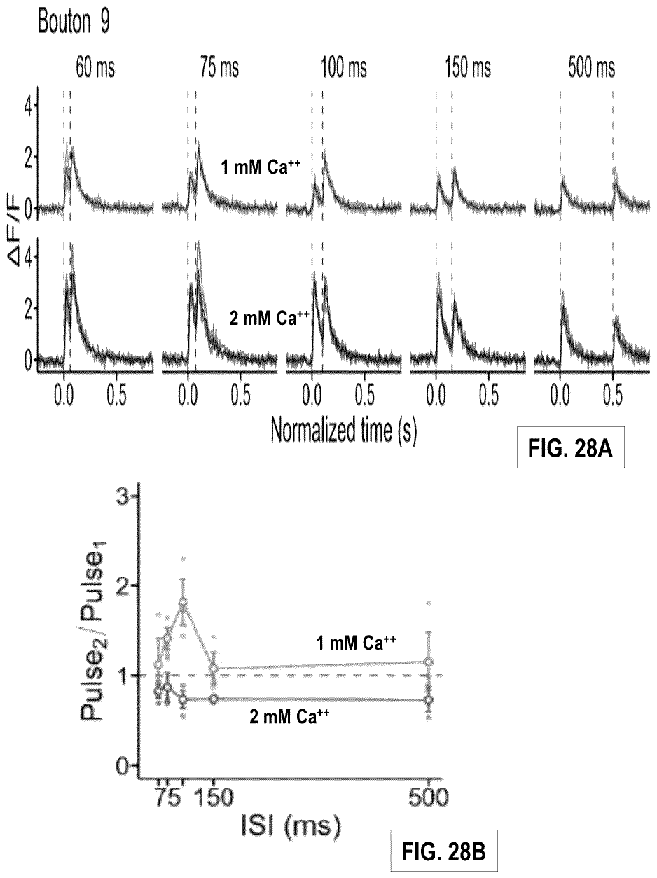

Some of the data presented here provides an interesting case study to the contrary. See FIG. 27A through FIG. 28B. FIG. 27A shows individual trial responses by [Ca⁺⁺]_{bath} with the averaged response in black. FIG. 27B shows PPR curves across ISIs for 1 mM and 2 mM Ca⁺⁺ for bouton 5. FIG. 28A shows individual trial responses color-coded by [Ca⁺⁺]_{bath} with the averaged response in black. FIG. 28B shows PPR curves across ISIs for 1 mM and 2 mM Ca⁺⁺ for bouton 9.

The magnitude of glutamate release (e.g., SVs released per stimulus) at these boutons was quite similar. Given that SVs released per stimulus strongly predicted functional class and RRP size, these boutons would probably fall into the same functional class. Nevertheless, their short-term plasticity behavior at 1 mM Ca⁺⁺ was strongly divergent, suggesting that these boutons possess distinct organizations of active zone machinery.

Notably, recent ultrastructural investigations used a high temporal resolution method, zap-and-freeze, to reveal that SVs transiently dock to the active zone membrane following stimulus. SVs remained docked for about 100 ms, potentially providing a mechanism by which presynapses could preferentially facilitate only at longer timescales. Whether the time constant for transient SV docking exhibits broad inter-bouton variation is unclear. These observations motivate structure-function investigations of synapse types with diverse short-term plasticity dynamics, which we anticipate will clarify how facilitation varies between synapses and may be utilized uniquely in different circuits.

iGluSnFR3 provides direct access to glutamatergic behavior at single synapses, but the transformational potential of this technology lies in combining iGluSnFR3 with other optical physiology reporters to access multiple physiological properties of the synapse simultaneously. For example, combining iGluSnFR3 with postsynaptic Ca⁺⁺ imaging at single dendritic spines could reveal the patterns of glutamatergic activity which favor NMDAR activation under physiological conditions. Toward this goal, we demonstrated that co-expression of iGluSnFR3 with spine-HaloTag enabled direct imaging of spontaneous glutamate release and its activation of NMDARs at single dendritic spines.

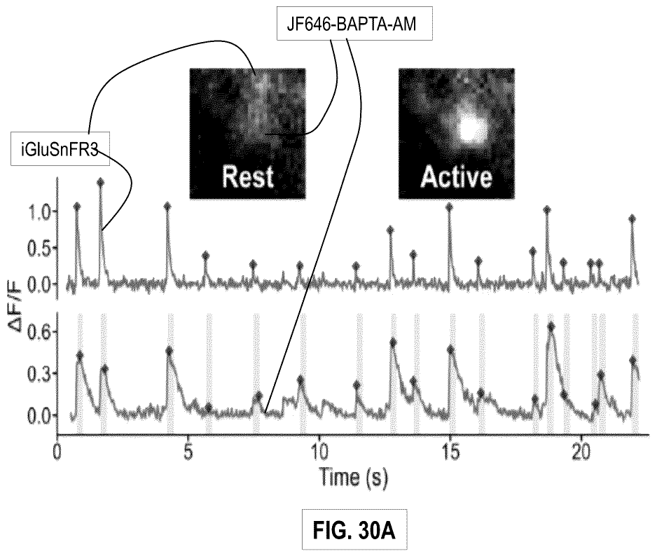

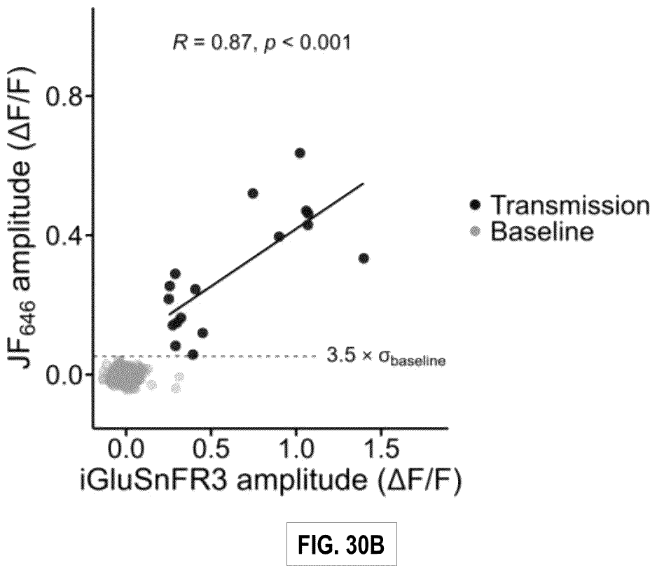

Larger glutamate release events triggered larger NMDAR-mediated Ca⁺⁺ flux at single spines, suggesting a dose-dependent effect of glutamate on the numbers of NMDARs activated. The small amplitude iGluSnFR3 events in FIG. 30A are in agreement with the spontaneous iGluSnFR3 events described herein, suggesting that single SV release only activates a subset of NMDARs at this spine. FIG. 30A presents a ΔF/F vs. time trace with spontaneous activity collected from iGluSnFR3 (green) and JF646-BAPTA-AM (magenta) from a single dendritic spine head. FIG. 30B is a scatterplot of JF646 peak amplitude vs. the iGluSnFR3 peak amplitude for the spine in FIG. 30A. The availability of NMDAR co-agonists like D-serine or glycine, or the NMDAR subtypes present at the synapse may play a role in this behavior.

Because these factors may be relevant to synaptic dysfunction in disease, high-throughput analysis of the heterogeneity of NMDAR activation across synapses will likely be particularly valuable. Ultimately, the high-throughput approach, according to several embodiments described herein, can improve imaging of synaptic function and opens new lines of inquiry for neuroscience. For example, therapeutics that target synapse function could be tested with our approach to determine their efficacy. At a more basic level, it could be used to understand the basic biophysics of NMDAR activation at single synapses. Deployed in brain slices, our approach could identify the patterns of NMDAR activation which trigger long-term potentiation at single synapses. Determining the heterogeneity of plasticity rules across synapse types and throughout brain development will drive novel insights into circuit function and its maturation.

This high-throughput approach captures many diverse aspects of synaptic functional heterogeneity, constituting a new field of synaptic functional “-omics.” We expect that synapse function-omics will have a broad impact in the study of neurological and neuropsychiatric illness where synaptic dysfunction is hypothesized but poorly understood. This in turn provides an objective measure of the efficacy or treatments, both in the trial and clinical stages.

### B. Specific Embodiments of the Invention

In one specific embodiment, a system is configured to receive data imaging data. The process involves several steps, optionally including a data collection step, an image segmentation step, an initial outlier identification step, at least one refinement step, and an output step.

These embodiments collectively highlight the versatility and scalability of the invention, providing a robust framework for analyzing synaptic function with high precision and throughput. The invention's ability to handle large data sets efficiently and its adaptability to different experimental conditions make it a valuable tool for advancing synaptic neuroscience and drug development.

### Example 1: General Methods and Materials

### 1A. DNA Constructs

pAAV.CAG.iGluSnFR3.v857.GPI was a gift from Kaspar Podgorski (Addgene plasmid #178335). pEF-Synaptophysin-mRuby was a gift from Edwin Chapman (Addgene plasmid #188980). LZF97_hSyn-spine-jRGECO1a was a gift from Don Arnold (Addgene plasmid #119198). psPAX2 (Addgene plasmid #12260) and pMD2.G (Addgene plasmid #12259) were gifts from Didier Trono. pFW_iGluSnFR3 was made by subcloning the promoter and open reading frame from pAAV.CAG.iGluSnFR3.v857.GPI into pFW using NEB HIFI Assembly. LZF97_hSyn-spine-HaloTag was made by replacing the jRGECO1a-TPR3-ZFBP sequence in LZF97_hSyn-spine-jRGECO1a with HaloTag using NEB HIFI Assembly. All sequences were confirmed by whole plasmid sequencing (Plasmidsaurus™) using Oxford Nanopore Technology™ with custom analysis and annotation.

### 1B. Lentivirus Production in HEK Cell Culture

Lentivirus was produced in HEK293T cells (ATCC CRL-3216) maintained in DMEM+10% FBS and penicillin/streptomycin at 37° C. and 5% CO₂. Cells were plated at 5×10⁶ cells/10 cm plate and transfected 12-24 hours later with 6 μg of either pFW_iGluSnFR3 or LZF97_hSyn-spine-HaloTag+4 μg psPAX2+2 μg pMD2.G using PEI for 4-6 hours. After 48 hours, the virus-containing media was harvested, debris removed by centrifugation at 1000 RPM for 5 min and 0.45 μm PES filtering, and single use aliquots were frozen at −80° C. for long term storage.

### 1C. Rat Hippocampal Neuron Culture and Transduction

All animal procedures were approved by the University of Maryland Animal Use and Care committee. Dissociated hippocampal cultures were prepared from E18 Sprague-Dawley rats of both sexes and plated on poly-L-lysine-coated coverslips (#1.5, 18 mm, Warner) at a density of 50,000 cells/coverslip according to methods known in the art. For experiments with iGluSnFR3 and Synaptophysin-mRuby, neurons were transfected with 1 μg of pAAV.CAG.iGluSnFR3.v857.GPI and 1 μg pEF-Synaptophysin-mRuby at DIV14-16 with Lipofectamine 2000 per manufacturer instructions. For experiments with iGluSnFR3 and spine-HaloTag, a subset of cells was infected with pFW_iGluSnFR3 and LZF97_hSyn-spine-HaloTag before plating, then plated along with uninfected cells. In this way, one could vary the ratio of infected cells to uninfected cells depending on the experimental requirements. We plated 10,000 dual-infected cells with 40,000 uninfected cells for a total density of 50,000 cells/well. Neurons were imaged between DIV17-23.

### 1D. Widefield and Confocal Microscopy

Widefield and confocal images were acquired on a Nikon TI2 inverted microscope equipped with an Andor Dragonfly spinning disk confocal, a Plan Apo 1D 60×/1.42 NA oil immersion objective. Excitation light (488/561/640 nm) was supplied by an Andor ILE and reflected to the sample through a 405/488/561/638 nm quadband polychroic (Chroma). Widefield and confocal images were acquired on a Nikon TI2 inverted microscope equipped with an Andor Dragonfly spinning disk confocal, a Plan Apo λD 60×/1.42 NA oil immersion objective. Excitation light (488/561/640 nm) was supplied by an Andor ILE and reflected to the sample through a 405/488/561/638 nm quadband polychroic (Chroma).

High-speed (200 Hz framerate) time-lapses of iGluSnFR3 activity were recorded in widefield mode, where the emission light bypassed the confocal unit to pass through appropriate emission filters (ET525/50, ET600/50, ET700/75 (Chroma)) to a Zyla 4.2+ sCMOS camera (Andor). 25.6×25.6 μm imaging regions were imaged at 20% laser power (488 nm, ˜8 W/cm²) with 5 msec exposures for experiments with iGluSnFR3 alone. For two-color experiments with iGluSnFR3 and spine-HaloTag, neurons were simultaneously illuminated with 488 and 640 nm laser lines. 51.2×51.2 μm imaging regions, with 2×2 pixel binning, were imaged at 30% laser power (488/640 nm, ˜11-13 W/cm2) with 20 msec exposures. The emission light was split by a 565 nm long-pass dichroic mirror to split the light from iGluSnFR3 and spine-HaloTag to two Zyla 4.2+ sCMOS cameras (Andor). For high-resolution confocal z-stacks, emission light was passed through the confocal unit to the appropriate emission filters to a Zyla 4.2+ sCMOS camera. Neurons were imaged in confocal mode at 50% laser power (488/561/640 nm, ˜1-2 W/cm²) with 200 msec exposures and 10 μm z-stacks (step size=0.3 μm) were acquired using a piezo-controlled stage (ASI).

### 1E. Glutamate Imaging

Cultured neurons co-expressing iGluSnFR3 and Synaptophysin-mRuby were imaged on DIV17-23. For imaging, neurons were transferred to an imaging chamber with parallel platinum electrodes spaced by about 1 cm and bathed in a modified Tyrode's buffer containing 136.5 mM NaCl, 3 mM KCl, 2 mM MgCl₂, 1 mM CaCl₂), 10 mM D-glucose, and 10 mM HEPES, at pH 7.4 (adjusted with 1 M NaOH), with 20 μM DNQX and 100 μM DL-AP5 to block recurrent excitation. When [Ca⁺⁺] was varied, NaCl was iso-osmotically substituted with CaCl₂) to maintain a nominal osmolarity of 308 mOsm. To help maintain temperature in the bath, the objective was heated to 37° C. with a heating collar (TOKAI HIT USA Inc., USA). In experiments with stimulus-evoked iGluSnFR3 activity, field stimuli (10 V/cm, 1 msec) were delivered by a stimulator box (S88X Square Pulse Stimulator, Grass Instrument Co.) triggered externally by a programmable stimulus generator (Master-8, A.M.P. Instruments, Israel).

In this method, 25.6×25.6 μm imaging regions with abundant iGluSnFR3+ axonal processes and Synaptophysin-mRuby puncta were selected and were subjected to a battery of stimulus protocols with 20-second rest periods in between protocols. Once the protocol sequence was complete, this was repeated at the next imaging region. When spontaneous iGluSnFR3 activity was imaged, the bath solution also contained 1 μM TTX to prevent APs. We repeatedly imaged single regions for 30 seconds at 200 Hz (5-second rest periods in between, 6-9 trials per region) to capture spontaneously released glutamate at individual boutons.

### 1F. Simultaneous Glutamate and Ca⁺⁺ Imaging

Cultured neurons co-expressing iGluSnFR3 and spine-HaloTag were prepared for imaging on DIV17-23. Spine-localized Ca⁺⁺ imaging was achieved by labeling spine-HaloTag with JF₆₄₆-BAPTA-HaloTagLigand (HTL)-AM. JF₆₄₆-BAPTA-HTL-AM was a gift from Luke Lavis. We aliquoted JF₆₄₆-BAPTA-HTL-AM as a 1 mM stock in anhydrous dimethyl sulfoxide (DMSO) and stored at −80° C.

To treat cells with JF₆₄₆-BAPTA-HTL-AM, a protocol similar to what has been previously described by Bradberry et al. 2021 was followed. Conditioned media (3×300 μL) was transferred from a well containing a coverslip with cultured neurons to an empty 12-well plate. For each experiment, a working stock of JF₆₄₆-BATPA-HTL-AM was diluted in a modified Tyrode's buffer containing 136.5 mM NaCl, 3 mM KCl, 2 mM MgCl₂, 1 mM CaCl₂, 10 mM D-glucose, 10 mM HEPES, and 2 μM JF₆₄₆-BAPTA-HTL-AM. For each coverslip incubation, 300 μL of the JF₆₄₆-BAPTA-HTL-AM working stock was added to a well with 300 μL of conditioned media, for a 1 μM effective dye concentration during incubation.

Coverslips of cultured neurons were transferred to the dye-containing wells and incubated for 30 minutes at 37° C. Following incubation, coverslips were immediately transferred sequentially to the other two wells with conditioned media to wash off free JF₆₄₆-BAPTA-HTL-AM before being returned to their original 12-well plate for recovery. Neurons were allowed to recover from the dye incubation for 30-60 minutes at 37° C. For imaging, neurons were transferred to an imaging chamber (Warner Instruments™) and bathed in a modified Tyrode's buffer containing 135 mM NaCl, 3 m KCl, 4 mM CaCl₂), 0 mM MgCl₂, 10 mM D-glucose, 10 mM HEPES, 1 mM Trolox (to preserve fluorescence of JF₆₄₆-BAPTA-HTL-AM), and 1 μM TTX in a climate-controlled chamber (37° C., 100% humidity, TOKAI HIT USA Inc.). Spontaneous glutamate release and subsequent NMDAR-mediated Ca⁺⁺ flux at single dendritic spines were observed for 20 seconds at 50 Hz before moving to another imaging region or washing in solution containing 100 μM DL-AP5. After wash-in, imaging of the same regions was repeated.

### 1G. Hardware Automation

A hardware automation scheme was developed to fully automate imaging experiments, improving throughput/reproducibility. A custom Python script enabled control of the microscope and stage (i.e. imaging positions), image acquisition, electrical stimulation, and solution exchange using a perfusion apparatus as described above with reference to steps 311 through 343.

The microscope and stage piezo were controlled from the script via the Andor (Oxford Instruments) REST API and Fusion software. This approach allowed us to store stage positions of multiple regions in a Python list object and iterate protocols over chosen imaging regions 192. We developed custom functions to automate updates to key imaging parameters (e.g. camera settings, filenames, focus stabilization) throughout the protocol, enabling more complex and robust automated imaging sequences.

In step 323, electrical field stimulation was controlled via the Master-8 and S88x Square Pulse Stimulator. At the beginning of each imaging protocol with electrical stimulus, we first established USB serial communication with the Master-8. To automatically update stimulus paradigms, a dictionary data structure was used to link stimulus paradigm parameters to imaging protocols. This enabled us to pre-configure the Master-8 prior to each imaging protocol to deliver diverse stimulus paradigms (e.g. single stimuli, paired stimuli, stimulus trains). To trigger stimulation during imaging, we predefined the frame in each imaging protocol (Fusion) during which a TTL pulse would be sent via BNC cable to the Master-8. After receiving the TTL from the microscope, the Master-8 triggered field stimulus of neurons by sending its pulse sequence via BNC cable to the S88X Square Pulse Stimulator.

In step 313, the workflow was similar for solution exchange. Sub-functions established USB serial communication with a ValveLink8.2 Controller (AutoMate Scientific™), which allowed toggle pinch valves on a gravity flow perfusion apparatus to operate, initiating fluid flow. To achieve fluid exchange, an Arduino microcontroller (Arduino, Italy) were simultaneously triggered which controlled a peristaltic pump, vacuuming excess fluid from the imaging chamber. The gravity flow perfusion apparatus was adjusted to a flow rate of ˜1.5 mL/min.

### 1H. Image Segmentation and Extraction of Intensity-Time Traces

These methods are described above with reference to step 403 and 404 of FIG. 4 and will not be repeated here.

### 1I. Fluorescence Signal Extraction

These methods are described above with reference to step 357 of FIG. 3 and steps 421 through 427 of FIG. 4 and will not be repeated here.

### 1J. Analysis of Single Stimulus-Evoked and Spontaneous iGluSnFR3 Transients

Annotated iGluSnFR3 transients from stimulus-evoked and spontaneous recordings were analyzed using custom analysis scripts in R. We measured the peak ΔF/F of found transients, and for stimulus-evoked transients, we calculated the time delay (Δt) between the stimulus and the peak ΔF/F to measure the speed with which iGluSnFR3 reports stimulus-secretion coupling. To measure the rise time, tᵣᵢₛₑ, we established a time cutoff of corresponding to the time of stimulus, prior to the time of peak ΔF/F. We then found the minimum ΔF/F prior to the peak maximum, and solved for the slope of a line between the minimum ΔF/F and the peak ΔF/F. From the peak ΔF/F, we established the 10% and 90% fluorescence levels for each transient and calculated the tᵣᵢₛₑ between these two points based on the found rise slope for each transient. To measure the decay time constant, τ_{decay}, for each iGluSnFR3 transient, we used the package nlstools to fit an exponential decay function, f(t)=Ae_{-t/τ} from the peak ΔF/F to the end of the annotated transient. From the calculated values for the pre-exponential factor, A, and τ_{decay}, we calculated the decay time from 90% to 10% of the peak ΔF/F, t_{decay}. Similarly, to calculate the full-width at half-maximum (t_{1/2}) of each transient, we used the 50% ΔF/F value for each peak to solve for the time of 50% ΔF/F on the peak rise (linear fit) and the peak decay (exponential decay fit), and subtracted these values to generate t_{1/2}. The procedure was identical for spontaneous iGluSnFR3 transients, except for the calculation of tᵣᵢₛₑ.

Since there was no stimulus to mark the beginning of an iGluSnFR3 transient, we established our time cutoff as 25 ms prior to the peak ΔF/F (informed by the published rise kinetics of iGluSnFR3, ˜19 ms). Otherwise, peak statistics were measured identically (omitting Δt, which could not be calculated). For both stimulus-evoked and spontaneous transients, signals were filtered out of the dataset if τ_{decay}<15 ms or τ_{decay}>300 ms, as we determined these represented spurious, high-frequency noise or non-glutamate release activity (e.g. iGluSnFR3+ vesicle trafficking through the axon, which had a characteristically slow time constant uncoupled from the stimulus), respectively.

Several properties of boutons were derived from our stimulus-evoked iGluSnFR3 trials. The probability of observing an iGluSnFR3 transient (P_{iGlu}) was defined as a binary for each trial: 1 if a signal was detected, or 0 if there was no signal detected. The sum of these binary values was divided by the total number of trials to define P_{iGlu} for an ROI at a single [Ca⁺⁺] bath.

To assess whether the stimulus-evoked glutamate release was mono- or multivesicular, we compared the average peak ΔF/F of stimulus-evoked iGluSnFR3 transients for each ROI (E_{ΔF/F}) to the average peak ΔF/F of spontaneous iGluSnFR3 transients at each [Ca2+]_{bath} (S_{ΔF/F}, see FIG. 3-FIG. 8 through FIG. 14), which represented putative single vesicle glutamate release events. When the value of the derived metric E_{ΔF/F}/S_{ΔF/F} was >1, we interpreted this as the release of multiple vesicles. The coefficients of variation (CVs, mean/σ) were calculated for all statistics at each ROI. For pair-wise comparisons, we calculated statistical significance via Kolmogorov-Smirnov and Mann-Whitney-Wilcoxon tests. To compare statistics across [Ca2+]_{bath}, we calculated one-way analysis of variance (ANOVA).

### 1K. Mean-Variance Analysis of iGluSnFR3 Transients

To perform mean-variance analysis (MVA) of iGluSnFR3 transients, we sampled the single action potential (AP)-evoked iGluSnFR3 activity at individual boutons across multiple [Ca2+]_{bath} (sampling multiple Pᵣ states). To accurately measure the mean and the variance of the iGluSnFR3 ΔF/F for each stimulus, we used an approach that was agnostic to the algorithmic definition of an iGluSnFR3 signal. The maximum of the iGluSnFR3 ΔF/F was measured in a time window starting from the stimulus (t=0) and extending to 250 ms post-stimulus.

This enabled measurement of sub-threshold maxima and noise, especially at 0.5 mM Ca⁺⁺, where most boutons were quiescent. We then converted the measured amplitudes to E_{ΔF/F}/S_{ΔF/F} and measured their variance. Each bouton possessed four data points of the form (x, y) corresponding to (mean, variance) at each [Ca⁺⁺]_{bath}, and each bouton was fit using the uniform probability binomial model of the form: σ2=Qx̅−x̅2/Nₛᵢₜₑₛ. In fitting the binomial model, Nₛᵢₜₑₛ was constrained to positive values <100 and Q was constrained to values between 0.8-1.2, as E_{ΔF/F}/S_{ΔF/F}=1 was expected to represent the iGluSnFR3 response due to a single vesicle, per the earlier analyses.

Boutons were accepted for analysis if the sum of the squared residuals was ≤1 and their maximum Pᵣ>0.45. From the binomial model, we calculated the following synaptic parameters: the uniform release probability (Pᵣ) at each [Ca2+]_{bath}, the quantal size of the iGluSnFR3 response (Q), and the total number of release sites in the readily releasable pool (Nₛᵢₜₑₛ).

### 1L. Generating a UMAP Representation of Bouton Physiology

To cluster boutons into functionally distinct classes (for example for the data reported in FIG. 17 through FIG. 19), we calculated a collection of statistics which summarized the intrinsic behavior of boutons during single AP trials. First, only iGluSnFR3 transients which exceeded our threshold of detection (see above, Fluorescence signal extraction) were used for calculating summary statistics. The probability of observing an iGluSnFR3 transient, P_{iGlu}, was the ratio of the number of transients identified at a given bouton to the number of single stimulus trials administered. Transients were only counted toward P_{iGlu} if a transient maximum occurred within 500 ms of a preceding stimulus. P_{iGlu} was calculated per bouton for each [Ca⁺⁺]_{bath}. We also calculated the mean amplitude and the variance for iGluSnFR3 transients in terms of E_{ΔF/F}/S_{ΔF/F} at each [Ca2+]_{bath}. Because the mean and variance were often unavailable for boutons which possessed no identifiable iGluSnFR3 transients at 0.5 mM Ca⁺⁺, boutons with NA were coerced to 0 for the purposes of the analysis.

For the remaining statistics, we restricted our analysis to [Ca2+]_{bath}=1, 2, or 4 mM, as data was too sparse to calculate coefficients of variation (CVs) at 0.5 mM Ca2+. We calculated the mean decay time constant, τ_{decay}, as well as the CV of peak ΔF/F, CV of τ_{decay}, and CV of Δt. The mean Δt was omitted as it exhibited little variation in magnitude across boutons (unlike mean amplitude and mean τ_{decay}).

For all statistics (except for P_{iGlu}, which was already normalized between 0 and 1), missing values were coerced to 0 and the values in each column were normalized between 0 and 1, equally weighting all of the statistics in the data matrix. This resulted in 24 data columns (each [Ca⁺⁺]_{bath} constituted an additional observation for each of the 7 summary statistics). We used the umap and dbscan libraries in R to generate UMAP scores for the data matrix and cluster the UMAP score output (DBSCAN minimum points per cluster=10). Cluster identities were re-associated with their original bouton IDs, which enabled us to categorize our bouton physiology data according to their UMAP class. Grouping the data in this way allowed us to visualize the defining characteristics of each bouton class.

### 1M. Analysis of iGluSnFR3 Transients Evoked by Paired Stimuli

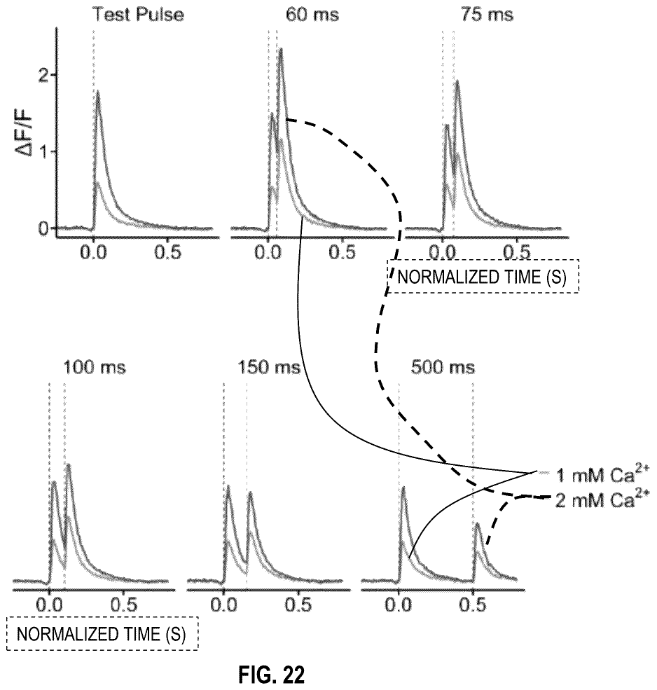

The protocol for the paired pulse experiment (see FIG. 22 through FIG. 28B) was administered with each protocol in triplicate. The protocol sequence began with a Test Pulse (a single stimulus), followed by paired pulses with inter-stimulus intervals (ISIs) of 60, 75, 100, 150, or 500 ms. There was a 20-second recovery period between each trial. This protocol sequence was first administered in a bath solution with 0.5 mM Ca⁺⁺. In between iterations of the protocol sequences, buffer solution containing 1 mM Ca⁺⁺ or 2 mM Ca⁺⁺ was washed in.

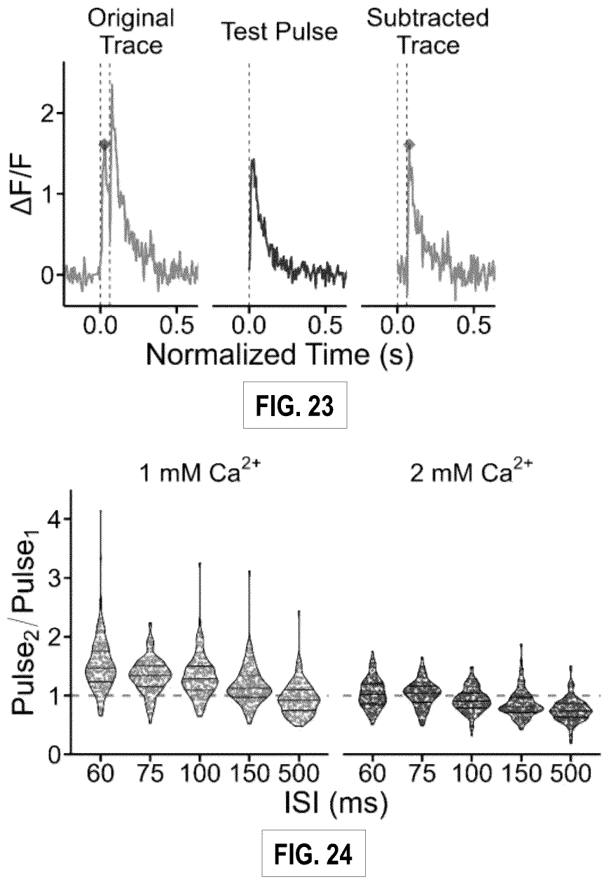

We averaged the responses from the three Test Pulse trials to generate the average response to a single stimulus for each ROI and each [Ca⁺⁺]_{bath} measured. We aligned the average response to the test pulse to the first stimulus in each paired pulse recording and subtracted the test pulse from the paired pulse recording, enabling more accurate measurement of the amplitude of the 2nd iGluSnFR3 transient (see FIG. 23). This was especially important in cases where a short ISI resulted in a convolution of the two iGluSnFR3 transients (e.g., ISI=60, 75, or 100 ms).

We performed this operation for every ROI in the dataset and then divided the traces into two stimulus epochs: the first stimulus epoch began at the first stimulus and ended at the second stimulus; the second stimulus epoch began with the second stimulus and terminated 500 ms later. For each trial, we identified the amplitude of the first pulse (Pulse₁) as the maximum ΔF/F within the first stimulus epoch of the unmodified trace. To determine the amplitude of the second pulse (Pulse₂), we identified the maximum ΔF/F within the second stimulus epoch in the subtracted trace. To eliminate noise from the measurement, we only retained amplitudes which exceeded the value of the average spontaneous transient amplitude. Paired pulse ratios were then calculated for each trial.

### 1N. Analysis of Simultaneous iGluSnFR3 and JF₆₄₆-BAPTA-AM Recordings

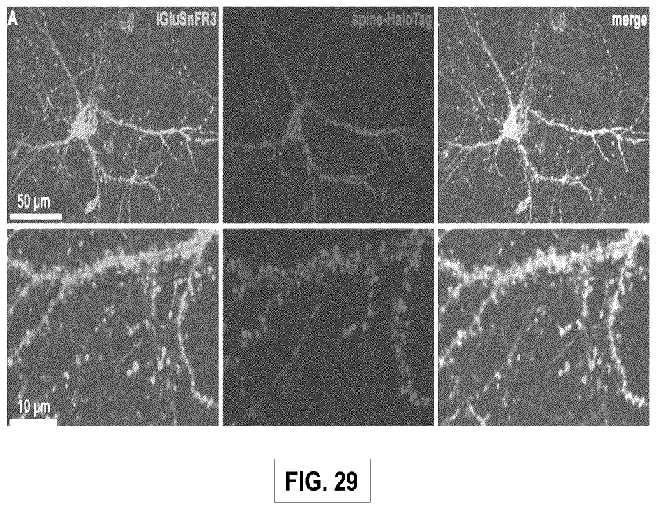

We segmented simultaneous recordings of iGluSnFR3 and JF₆₄₆-BAPTA-HTL-AM on the activity in the iGluSnFR3 channel as described above. These iGluSnFR3 activity maps were distributed to folders containing the recording for either the iGluSnFR3 or JF₆₄₆ channel and intensity-time traces were extracted as before. Because the ROI maps were identical between channels of the same recording, we were able to pair the traces by ROI in R. To characterize putative synaptic transmission events (paired iGluSnFR3/JF₆₄₆ signals), we took advantage of our robust peak discrimination in the iGluSnFR3 channel. Around each iGluSnFR3 signal identified by our peak finding algorithm, we defined a temporal window in which to search for a local maximum in the JF₆₄₆ channel (see FIG. 29 through FIG. 35). The lower boundary of the temporal window was 10 ms prior to the iGluSnFR3 peak maximum, and the upper boundary of the temporal window was 250 ms after the iGluSnFR3 peak maximum. In this way, we were able to pair the local maxima (which reflected the peak of paired Ca⁺⁺ transients) in each channel for bona fide glutamate release events, which could then be analyzed.

To discriminate putative transmission events from noise, we took advantage of the output from our peak finding algorithm, which tabulates putative baseline indices as part of the baseline adjustment routine. For each dendritic spine in the dataset, we selected all of the putative baseline indices for the JF₆₄₆-BAPTA channel and calculated the standard deviation of these points. A signal threshold of 3.5a was set for each spine, and JF₆₄₆-BAPTA signals above this threshold were considered transmission.

### 1O. Data Visualization

All data analysis and visualization was carried out using custom scripts in R. In general, we relied on structured data frames with several string variables by which data could be sorted and analyzed. Figures were plotted using a combination of the packages ggplot2 and gridExtra. For the maps of synaptic function, we used a custom workflow and the packages RImageJROI and EBImage. First, we generated .png files of the max-projected confocal z-stacks for individual imaging regions. These .png files were read into R as raster objects. Zip files containing the ROI information were generated for each imaging region in FIJI. Using RImageJROI, we were able to read the .zip files into R. Using ggplot2, we layered the ROI polygons from FIJI on top of the raster of the imaging region. Statistics could then be assigned to each ROI polygon on the basis of their unique ROI identity.

### 1P. Statistics

Statistical analyses were performed in R. Statistical differences between datasets were tested with pair-wise Mann-Whitney-Wilcoxon and Kolmogorov-Smirnov tests. For datasets with multiple groups, data were also tested with one-way ANOVA.

### Example 2: High-Throughput Framework for Analyzing Glutamate Release from Single Boutons with iGluSnFR3

We sought to leverage iGluSnFR3 to characterize the heterogeneous glutamate release properties of individual presynaptic boutons in cultured rat hippocampal neurons. To accelerate experimental throughput and improve reproducibility, we devised an automated framework for efficiently collecting large datasets (FIG. 1 through FIG. 4). We used microscopy software from Andor (Fusion) and their Python-accessible REST API to communicate with our microscope. See FIG. 1A, which shows the automated imaging approach using custom Python scripts. This process enables high-throughput optical physiology of presynaptic boutons. Python routines control the microscope, triggering imaging protocols, and controlling stage positions via a REST API (Andor™, Oxford Instruments™). Python sub-routines configure a programmable stimulus generator. Whenever an imaging protocol triggers a TTL pulse to the programmable stimulus generator, the pre-configured stimulus paradigm is sent to an electrical stimulator which stimulates neurons via parallel platinum wire electrodes. Other Python sub-routines toggle pinch valves on a gravity flow perfusion apparatus simultaneously with an Arduino-controlled vacuum to permit fluid exchange. The result is a versatile, automated approach to experimental design that dramatically improves reproducibility and throughput for assays of synapse physiology with iGluSnFR3. At the right are imaging regions of axons expressing iGluSnFR3 (gray LUT) and Synaptophysin-mRuby (fire LUT), a marker for the presynaptic bouton. Scale bar=5 μm.

With the REST API, we wrote imaging protocols that captured stimulus-evoked or spontaneous iGluSnFR3 activity with widefield imaging along axonal arbors at 200 Hz, followed by high-resolution, confocal z-stacks for an arbitrary number of imaging positions. Custom Python scripts allowed flexible, fully automated control of both electrical stimulation and solution exchange, enabling the design of versatile experiments to probe the physiological properties of presynaptic boutons using iGluSnFR3.

To capture iGluSnFR3 activity localized to presynaptic boutons, we transfected hippocampal neurons with iGluSnFR3 and Synaptophysin-mRuby, a fluorescent marker which labels the synaptic vesicle (SV) pool at boutons. We used a custom ImageJ macro based on the AI-based ImageJ plugin SynQuant™ to batch segment Synaptophysin-mRuby puncta within the boundary of iGluSnFR3+ axonal arbors. We applied these maps of regions-of-interest (ROIs) to our iGluSnFR3 timelapses (FIG. 2A), tracking single boutons across many imaging rounds. FIG. 2A shows time-lapse recordings of iGluSnFR3 activity along axons can be batch segmented based on iGluSnFR3 activity. Scale bar=10 μm.

Alternatively, we could batch segment videos using iGluSnFR3 activity itself by modifying a MATLAB script developed previously. See FIG. 2B, which shows time-lapse recordings of Synaptophysin-mRuby. The ROI based on merging these two signals is shown in FIG. 2C. Activity-based segmentation (e.g., signals in FIG. 2F and ROIs FIG. 2G) was useful for expediting analysis (our implementation did not rely on pre-processing steps, e.g. drift correction) or in situations where a synapse marker could not be co-expressed. However, activity-based segmentation also fails to capture inactive synapses, biasing subsequent statistical measures of presynaptic function. Despite its requirement for more image processing, we chose to use a fluorescent marker to track synapses where possible.

We frequently identified 20 or more ROIs along axonal arbors in single imaging regions (25.6×25.6 μm). In some experiments, we recorded the same imaging region 192 more than 40 times, resulting in >800 intensity-time traces of iGluSnFR3 activity to be analyzed per region. With multiple regions 192 in a single experiment, we required a scalable solution for extracting relevant iGluSnFR3 activity for analysis. As described above with reference to FIG. 4, we developed a custom algorithm in R based on a median filter, which automatically corrects baseline fluctuations due to noise and identifies iGluSnFR3 signals caused by stimulus-evoked and spontaneous glutamate release at single boutons (FIG. 2D). Briefly, a rolling median filter was used to approximate the baseline and identify intensity outliers in top traces for both experiments (stimulated, aka evoked, and spontaneous). Left, stimulus-evoked iGluSnFR3 activity; right, spontaneous, i.e., AP-independent iGluSnFR3 transients. Outlier detection was refined in subsequent steps to estimate where putative iGluSnFR3 transients occurred in the second trace for each experiment, and these putative signals were excluded from a smoothed estimate of the baseline passing through the noise in the second trace of each experiment. ΔF/F in the fourth trace is based on deviations from the smoothed baseline The algorithm's output for two examples of regions-of-interest (ROIs) are shown, with a stimulation pulse identified between 1 and 2 seconds in the first experiment, and five temporally confined peaks in the second experiment.

From the baseline-corrected trace, we extracted iGluSnFR3 transients for downstream analysis using a signal threshold of 5σ, where σ is the standard deviation of the noise. FIG. 2E shows an example of a stimulus-evoked iGluSnFR3 transient and the intrinsic peak parameters we analyzed: peak amplitude (peak ΔF/F), fitted exponential decay time constant (τ_{decay}), the full-width at half-maximum (t_{1/2}), 10-90% rise time (tᵣᵢₛₑ), 90-10% decay time (t_{decay}), and the time-to-peak following electrical stimulation onset (Δt).

### Example 3: Batch Segmentation Methods Extract Quantal, Spontaneous iGluSnFR3 Activity Localized to Boutons

Action potential (AP)-independent, spontaneous glutamate release events should localize to putative presynaptic boutons marked by Synatophysin-mRuby. To test whether our automated approach could capture this, we imaged spontaneous iGluSnFR3 activity at axonal arbors co-expressing iGluSnFR3 and Synaptophysin-mRuby in a modified Tyrode's buffer containing 1 μM tetrodotoxin (TTX) and either 0.5, 1, 2, or 4 mM Ca⁺⁺ ([Ca⁺⁺]_{bath}), segmenting recordings according to Synaptophysin-mRuby expression or iGluSnFR3 activity. In some embodiments, buffers were automatically introduced using the chemical perfusion apparatus 154.

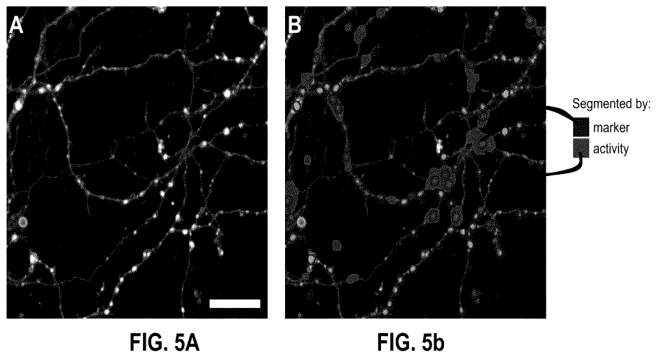

FIG. 5A shows an example of imaging area 192 (51.2×51.2 μm) with an axon co815 expressing iGluSnFR3 (grey LUT) and Synaptophysin-mRuby3 (fire LUT). Scale bar=10 μm. FIG. 5B is a spatial map of batch segmented ROIs according to Synaptophysin-mRuby3 expression or detected iGluSnFR3 activity (dark) at [Ca⁺⁺] bath=2 mM for one trial. This figure shows the boutons (dark) collected by our fluorescent marker segmentation compared to activity-based segmentation (grey) for a single imaging trial. The segmentation comparison indicates that several boutons marked by Synaptophysin-mRuby3 encompass true glutamate release sites, though many more boutons tended to be silent during the 30 s recording period. Notably, while activity segmentation appears to merge the responses of multiple boutons in some instances, the strong concordance of activity ROIs with boutons expressing Synaptophysin-mRuby suggests that both methods can capture iGluSnFR3 activity localized to single boutons.

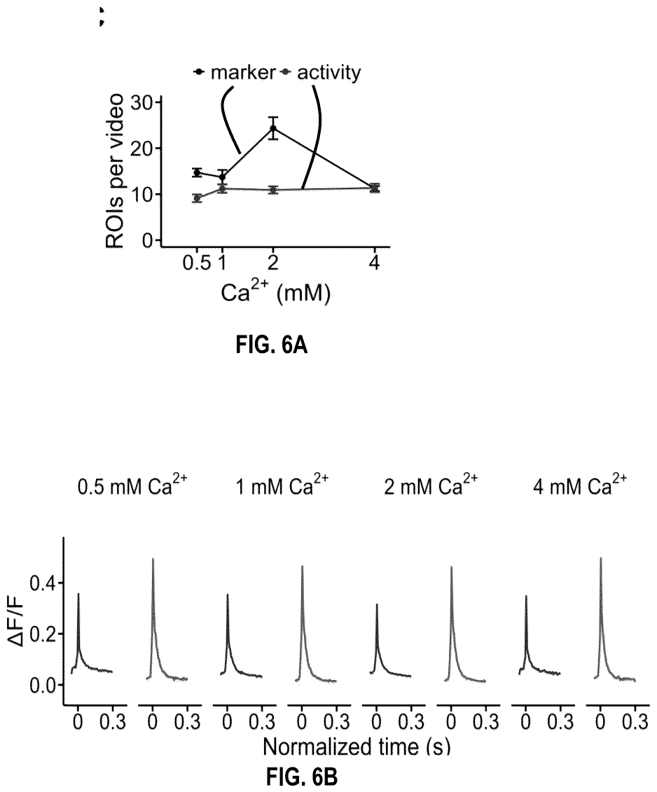

FIG. 6A shows data for the number of ROIs detected per imaging region with each segmentation method. It is evident that the lower number of ROIs collected by activity segmentation reflects that not every bouton will spontaneously release glutamate during the imaging period (30 seconds). However, we also note that our implementation of activity segmentation relies on thresholding activity maps on the top 3% of all pixel intensities. Boutons with low signal-to-noise ratio (SNR) can escape detection (see FIG. 2D), which may underestimate the number of active boutons.

FIG. 6B shows data for the average AP821 independent, spontaneous iGluSnFR3 transients at each [Ca⁺⁺] bath for either segmentation method, marker first then activity. The average iGluSnFR3 response collected by our high-throughput approach showed little variation in iGluSnFR3 transient shape across [Ca⁺⁺] bath when compared within segmentation method (FIG. 2D-6B), reflecting that spontaneous iGluSnFR3 transients are attributable to glutamate release from single SVs at all [Ca⁺⁺] bath. Activity-based segmentation always resulted in a larger average iGluSnFR3 response, likely because activity segmentation captures the site of maximal iGluSnFR3 signal, which can be interpreted as the putative SV release site. The figure shows the average AP821 independent, spontaneous iGluSnFR3 transients at each [Ca⁺⁺] bath for either segmentation method. For both methods, the shape and amplitude of the average iGluSnFR3 transient was similar across all [Ca⁺⁺] bath, supporting the notion that spontaneous iGluSnFR3 activity represents single vesicle release. By contrast, marker-based segmentation may be subject to orientation effects that reduce SNR, such that the maximal iGluSnFR3 signal could be offset from the SV cloud marked by Synaptophysin-mRuby (visible in top-right, FIG. 5B).

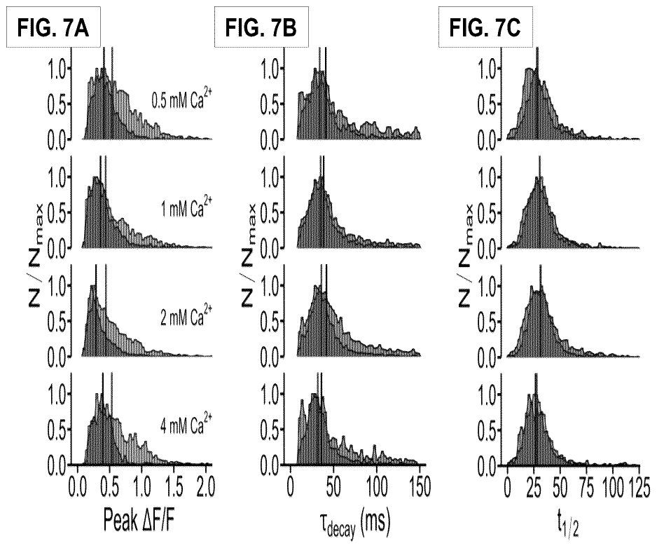

FIG. 7A through FIG. 7C show the normalized histograms for selected iGluSnFR3 event parameters across [Ca⁺⁺]_{bath} and segmentation method (dark by marker, grey by activity. (FIG. 2E-7A: Normalized histogram of peak ΔF/F collected via marker or activity segmentation across [Ca⁺⁺] bath. FIG. 7A shows a normalized histogram of peak ΔF/F collected via marker or activity segmentation across [Ca⁺⁺] bath. FIG. 7B shows a normalized histogram of τ_{decay}. FIG. 7C shows a normalized histogram of t_{1/2}. Vertical lines indicate the medians of the distributions. Detected iGluSnFR3 transients were persistently larger in amplitude with faster τdecay when extracted via activity segmentation.

None of the distributions of peak parameters varied substantially in magnitude across [Ca⁺⁺] bath when compared within segmentation method. Comparing our segmentation approaches, activity segmentation produced iGluSnFR3 events with larger amplitudes and smaller τ_{decay}, supporting the conclusion that activity segmentation produces ROIs more likely to capture true glutamate release hotspots. However, the data presented here also indicate that Synaptophysin-mRuby segmentation reliably captures quantal activity at putative single boutons. For our subsequent experiments, we used Synaptophysin-mRuby segmentation, which enabled us to track bouton behavior even when boutons were inactive.

### Example 4: Estimating the Number of Synaptic Vesicles Released During Evoked Transmission

The number of SVs released during an AP is a critical determinant of the strength of transmission at single synapses. This can be assessed at single synapses using SV-targeted pHluorins to directly count released SVs and measure the Pᵣ of SV release sites at single boutons following an AP. Conversely, the total number of fusion-competent SVs available to be released during an AP, the readily releasable pool (RRP), can be measured using mean-variance analysis (MVA). MVA is typically applied to electrophysiology recordings to calculate the size of the RRP through the relationship I=Nₛᵢₜₑₛ×Pᵣ×Q, where I is the mean amplitude of the postsynaptic receptor current, Nₛᵢₜₑₛ is the total number of release sites in the RRP, Pᵣ is the uniform release probability for the SVs in the RRP, and Q is the current associated with a single SV. However, electrophysiology generally measures presynaptic properties of ensembles of synapses, whereas single-synapse resolution requires fortuitous circuit architecture. Optical sensors which directly monitor glutamate release (e.g. iGluSnFR3) go beyond pHluorin imaging or electrophysiology, accessing both the relative magnitude of glutamate release during a single AP and the total size of the RRP through MVA with single-synapse resolution.

Since iGluSnFR3 is sensitive to putative single SV release events, we reasoned that our high throughput approach should be able to determine both how many SVs are released per AP and the overall size of the RRP, enabling determination of the RRP fraction mobilized during each AP at single bouton resolution. To achieve this, we imaged the iGluSnFR3 response evoked by a single AP (10 trials, 20 seconds between trials) at boutons labeled by Synaptophysin-mRuby (n=244 boutons) across different [Ca⁺⁺] bath (0.5, 1, 2, and 4 mM).

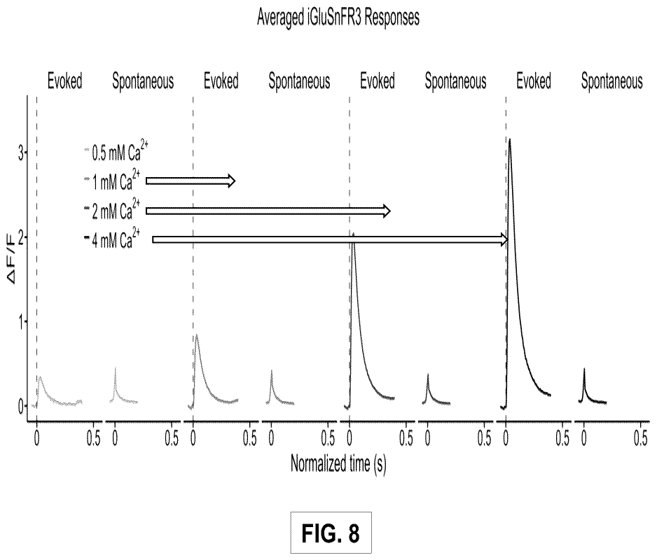

FIG. 8 shows the averaged iGluSnFR3 response for stimulus-evoked and spontaneous glutamate release across [Ca⁺⁺]bath=0.5, 1, 2, or 4 mM. FIG. 8 compares the average stimulus-evoked response to the average spontaneous response (spontaneous data corresponds to marker-based segmentation data from FIG. 5 through FIG. 7). Dashed vertical lines indicate stimulus.

The amplitude of evoked iGluSnFR3 transients increased substantially with increasing [Ca⁺⁺]_{bath} as more SVs were released during each AP, in contrast to spontaneous iGluSnFR3 transients, which did not increase in amplitude.

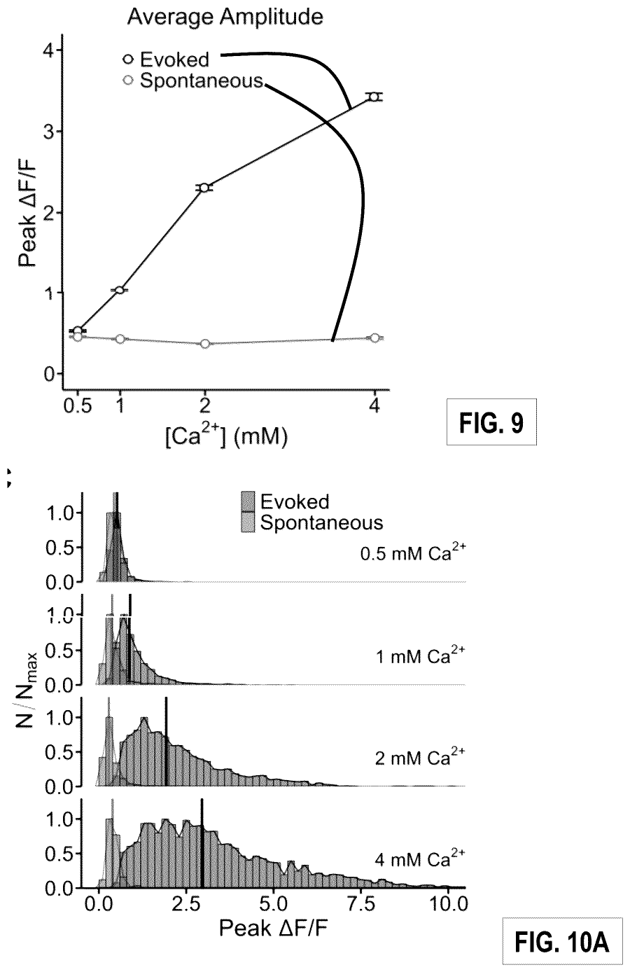

FIG. 9 shows the scatterplot of average peak amplitude (peak ΔF/F) vs. [Ca²⁺] bath for stimulus evoked and spontaneous iGluSnFR3 transients. This shows the dramatic increase in evoked response while the spontaneous response stay rather constant as [Ca²⁺] concentration increases.

FIG. 10A is a normalized histogram of peak ΔF/F comparing identified spontaneous (light) with stimulus-evoked (dark) iGluSnFR3 transients at each [Ca⁺⁺] bath. FIG. 10B is a normalized histogram of τ_{decay} comparing spontaneous (light) with stimulus-evoked (dark). From the normalized histograms of peak ΔF/F and τ_{decay}, we observed that the statistics for evoked iGluSnFR3 events at [Ca⁺⁺] bath=0.5 mM exhibited substantial overlap with their spontaneous counterparts, suggesting boutons only release a single SV per AP at this [Ca⁺⁺] concentration. Most boutons transitioned to releasing multiple SVs per stimulus as [Ca⁺⁺] concentration increased. See FIG. 3C-10A, a normalized histogram of peak ΔF/F comparing identified spontaneous (orange) with stimulus-evoked (black) iGluSnFR3 transients at each [Ca⁺⁺] bath. At 2 and 4 mM Ca⁺⁺, boutons transitioned to multivesicular release. In FIG. 10A and FIG. 10B, vertical lines indicate median values of the distributions for stimulus evoked and spontaneous iGluSnFR3 transients, respectively.

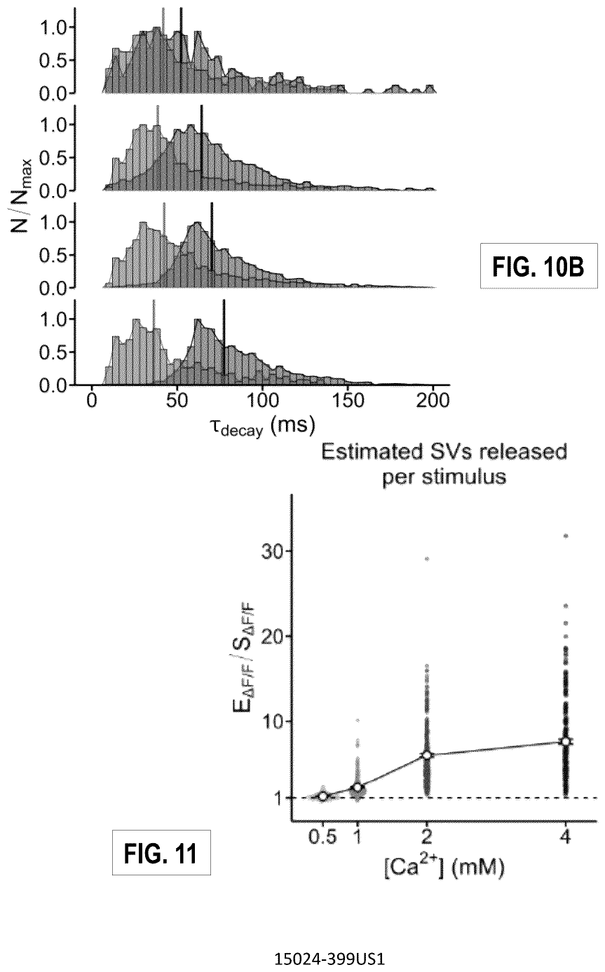

To count the SVs released per AP, we normalized the peak ΔF/F of each evoked iGluSnFR3 response (E_{ΔF/F}) to the population average of the spontaneous iGluSnFR3 responses (S_{ΔF/F}). FIG. 11 is a scatterplot of the ratio of evoked response (E_{ΔF/F}) to spontaneous response (S_{ΔF/F}) i.e., E_{ΔF/F}/S_{ΔF/F} vs. [Ca⁺⁺] bath. E_{ΔF/F}/S_{ΔF/F} is the peak ΔF/F of stimulus-evoked iGluSnFR3 transients (E_{ΔF/F}) normalized to the population average of the peak ΔF/F of spontaneous iGluSnFR3 transients (S_{ΔF/F}) at each respective [Ca⁺⁺]_{bath}. Dots indicate the mean value of (E_{ΔF/F}/S_{ΔF/F} at individual boutons. This figure shows the average value of E_{ΔF/F}/S_{ΔF/F} vs. [Ca⁺⁺]_{bath}, where each colored point is the average response of a single bouton. At 0.5 mM Ca2+, the few boutons that were active released approximately 1 SV (E_{ΔF/F}/S_{ΔF/F}=1.12±0.01), while at 4 mM Ca⁺⁺, boutons released approximately 8.5 vesicles per AP (E_{ΔF/F}/S_{ΔF/F}=8.48±0.32). The responses at 0.5 mM Ca²⁺ cluster around E_{ΔF/F}/S_{ΔF/F}=1 (horizontal dashed line), indicating univesicular release at this Ca⁺⁺.

Ultrastructural observations of boutons in cultured mouse hippocampal neurons indicate that there are 10.1±4.3 (mean±s.d.) docked SVs per active zone, suggesting that at 4 mM Ca⁺⁺, most boutons in our sample release >80% of the SVs in the RRP. However, some boutons released >20 putative SVs per AP at 4 mM Ca⁺⁺, implying that a subset of boutons in our sample either experienced synaptic crosstalk or possessed multiple active zones.

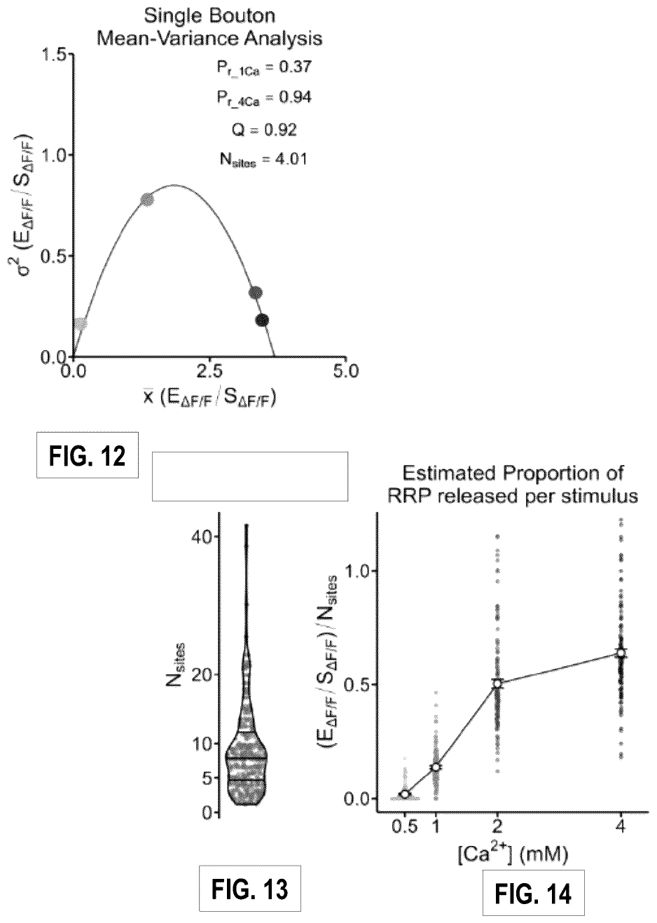

To better understand what fraction of the RRP was mobilized per AP at individual boutons, we fit the mean (x̅) and variance (σ2) of the E_{ΔF/F}/S_{ΔF/F} values for each bouton with a uniform probability binomial model, σ2=Qx̅−x̅2/Nₛᵢₜₑₛ. FIG. 12 a scatterplot of variance (σ2) of E_{ΔF/F}/S_{ΔF/F} vs. mean (x̅) of E_{ΔF/F}/S_{ΔF/F} in an example of a bouton. Mean-variance distributions were fit with a binomial of the form σ2=Qx̅−x̅2/Nsites, where Q is the single vesicle iGluSnFR3 response and Nsites is the total number of release sites at each bouton (i.e. the readily releasable pool, RRP). From the fitted binomial, we extracted the uniform release probability at each release site, Pr, for each [Ca⁺⁺]_{bath}. The Pr for 1 and 4 mM Ca⁺⁺, as well as the solved values for Q and Nsites are shown on the plot.

Of the 244 boutons in our sample, 122 possessed mean-variance behavior that was well described by the model, yielding an average RRP size of Nₛᵢₜₑₛ=9.05±0.63. FIG. 13 is a violin plot of the distribution of solved Nsites per bouton. This plot is in good agreement with previous ultrastructural measurements and within the range of Nₛᵢₜₑₛ (1-10) observed by other glutamate imaging methods. The behavior of 122/244 boutons in our dataset could be well-described by a binomial fit. Of these 122 boutons, most were quiescent at 0.5 mM Ca⁺⁺ (E_{ΔF/F}/S_{ΔF/F}=0.101±0.013), but at 1, 2, and 4 mM Ca⁺⁺ boutons released 1.11±0.08, 3.89±0.22, and 4.98±0.25 SVs, respectively. When we compared estimated SV release (e.g., mean E_{ΔF/F}/S_{ΔF/F}) to RRP size at each bouton at 1, 2, and 4 mM Ca⁺⁺, boutons mobilized 13.7%, 50.4%, and 63.8% of their total RRP, respectively. FIG. 14 is a scatterplot of E_{ΔF/F}/S_{ΔF/F}/Nsites vs. [Ca⁺⁺]_{bath}. As the value of E_{ΔF/F}/S_{ΔF/F} was a proxy for the total number of vesicles released per stimulus, the ratio of E_{ΔF/F}/S_{ΔF/F} to Nsites permitted us to estimate the proportion of the RRP mobilized per stimulus at each bouton across [Ca⁺⁺]_{bath}.

Taken together, our method extends previous approaches by directly comparing the RRP size calculated from MVA with the approximate number of SVs released per AP at single boutons, permitting a direct assessment of presynaptic efficacy. This approach can provide further access to the time constants associated with vesicle recycling and release site replenishment at individual boutons by measuring how the fraction of RRP released per AP changes with stimulus frequency.

### Example 5: Tracking the Glutamate Release Behavior of Single Boutons Reveals Three Classes of Boutons

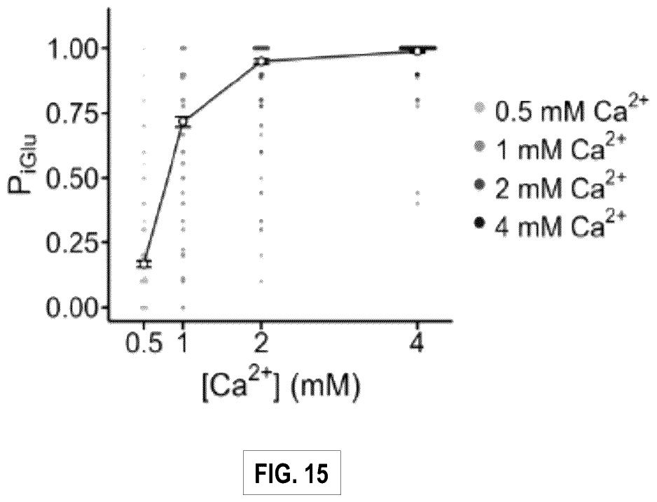

The high information content of our iGluSnFR3 recordings suggested that boutons could be classified according to their stimulated iGluSnFR3 response behavior. We examined a set of bouton features for each of the 244 boutons in our sample to characterize their iGluSnFR3 behavior in response to a single AP. First, we calculated the probability of observing stimulus-evoked iGluSnFR3 activity (P_{iGlu}) for each bouton across [Ca⁺⁺] bath. FIG. 15 is a graph showing the probability of stimulus-evoked iGluSnFR3 transients (PiGlu).

At [Ca⁺⁺]_{bath}=0.5 mM, most boutons were quiescent (P_{iGlu}=0.16±0.01), but P_{iGlu} increased sharply at [Ca⁺⁺]_{bath}=1 mM (P_{iGlu}=0.68±0.02). At [Ca⁺⁺]_{bath}=4 mM, almost all boutons exhibited iGluSnFR3 activity (P_{iGlu}=0.96±0.01), suggesting nearly all boutons in our sample were glutamatergic or near enough to a glutamatergic synapse to experience glutamate spillover during periods of strong glutamate release.

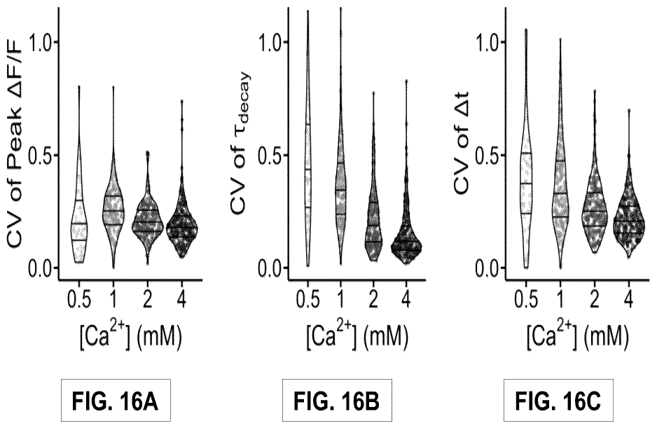

We also calculated the coefficient of variation (CV) across trials for intrinsic peak parameters, including peak ΔF/F for measured iGluSnFR3 transients. FIG. 16A is a plot showing the coefficient of variation (CB) of the peak ΔF/F. for measured iGluSnFR3 transients. FIG. 16B and FIG. 16C are plots showing the CV of τ_{decay} and the CV of Δt, respectively. For Δt and τ_{decay}, the interquartile range (IQR) of the CV distributions decreased as [Ca⁺⁺]_{bath} increased, indicating that the onset and decay kinetics of evoked iGluSnFR3 responses between trials became more uniform with increasing Ca⁺⁺ availability for each bouton. Each dot in the violin plots indicates the value of CVΔF/F for a single bouton. The trend for CV of peak ΔF/F (CV_{ΔF/F}) was more complex. At 0.5 mM Ca⁺⁺, the few boutons that were active could only release a single SV following stimulation (see FIG. 12, above), biasing CV_{ΔF/F} toward small values. By contrast, at all other [Ca⁺⁺]_{bath} we observed a broad range of CV_{ΔF/F} values, suggesting the number of SVs released at individual boutons varied between stimuli.

Adding these statistics together with the mean amplitude and variance of the iGluSnFR3 response at each [Ca⁺⁺]_{bath}, we thus compiled 24 features for each bouton that could be analyzed for their relationship to one another using available software from uniform manifold approximation and projection (UMAP). FIG. 17 is a UMAP representation of statistics of iGluSnFR3 activity at 251 boutons. there are three clusters of points indicative of three classes of boutons as determined using a DBSCAN routine of the UMAP library.

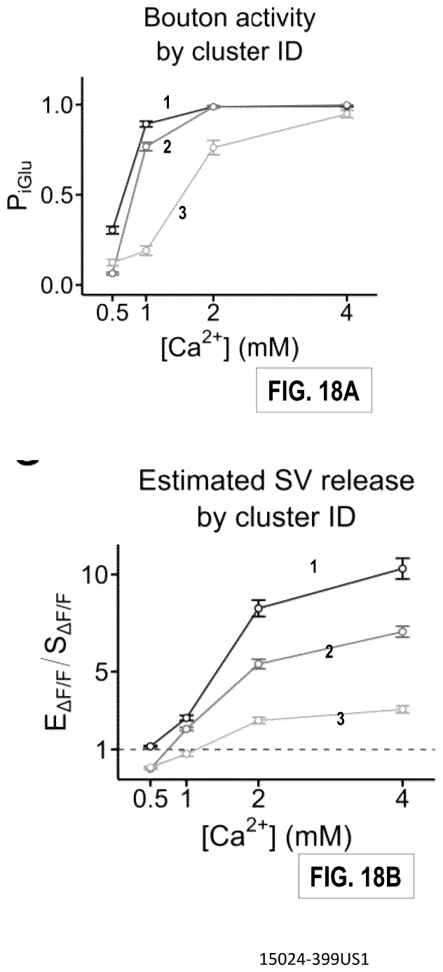

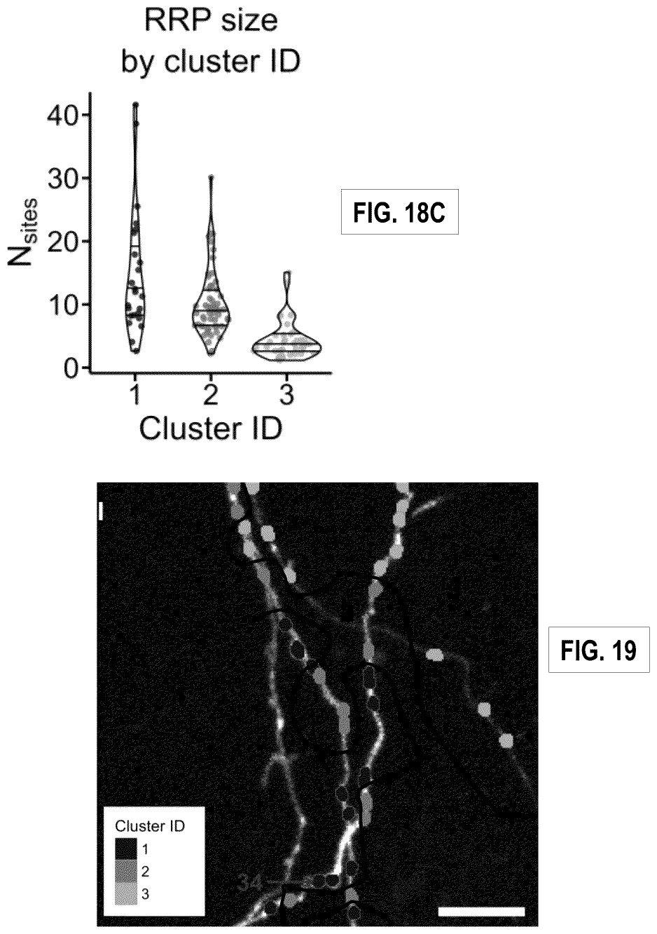

FIG. 18A shows data for P_{iGlu} vs. [Ca⁺⁺]_{bath}, grouped by cluster ID (1, 2, 3). These curves reveal that Class 1 boutons were almost always active at [Ca⁺⁺]_{bath}=1 mM (P_{iGlu}=0.89±0.02), while Class 3 boutons were only active once every 5 stimuli (P_{iGlu}=0.19±0.03).

FIG. 18B shows the estimated number of synaptic vesicles (SVs) released (E_{ΔF/F}/S_{ΔF/F}) vs. [Ca⁺⁺]_{bath}. When SVs were in their lowest Pᵣ state at 0.5 mM Ca⁺⁺, active Class 1 boutons reliably released a single SV (E_{ΔF/F}/S_{ΔF/F}=1.16±0.04) while Class 2 and 3 boutons were categorically quiescent. When SVs were in their highest Pᵣ state at 4 mM Ca⁺⁺, Class 1 boutons released ˜10 SVs, >3-fold more than Class 3 boutons (Class 1: E_{ΔF/F}/S_{ΔF/F}=10.3±0.5, Class 3: E_{ΔF/F}/S_{ΔF/F}=3.1±0.2). Thus, large-scale functional analysis is suitable to classify synapses based purely on presynaptic release characteristics, highlighting the utility of the imaging and analysis pipeline.

The methods used here also can incorporate other non-functional characteristics into UMAP classification, e.g. intensity of the fluorescent presynaptic marker or region size. By grouping the data according to cluster identity, we identified defining features of each bouton grouping:

To examine the basis of the observed functional classification, we mapped the cluster IDs onto the bouton parameters extracted from MVA. The distribution of Nₛᵢₜₑₛ according to cluster ID suggested that the fidelity and magnitude with which Class 1 boutons responded to stimuli could be explained by their larger RRPs. FIG. 18C shows the estimated RRP size (Nsites) vs. cluster ID. For Class 1: Nₛᵢₜₑₛ=14.7±2.). These large RRPs imply Class 1 boutons possess multiple active zones.

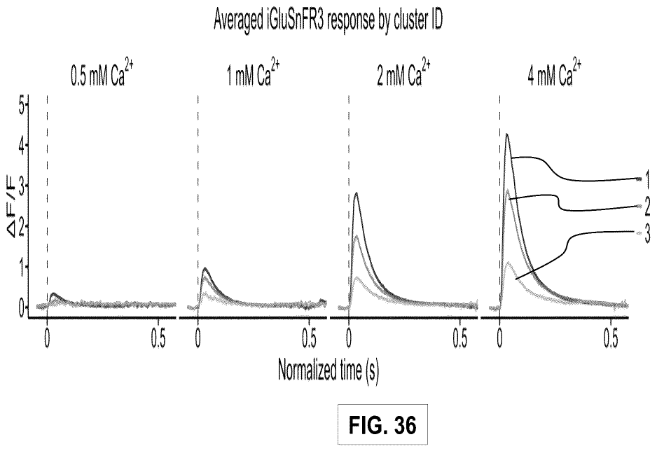

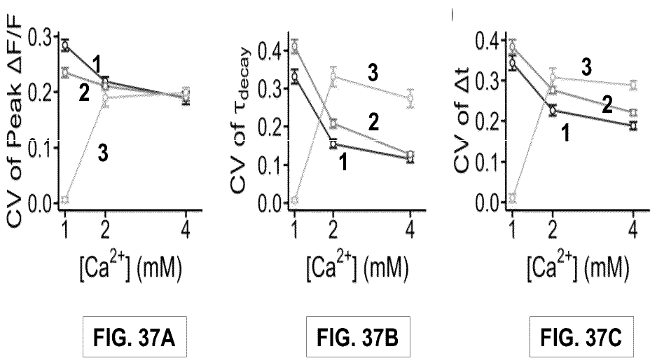

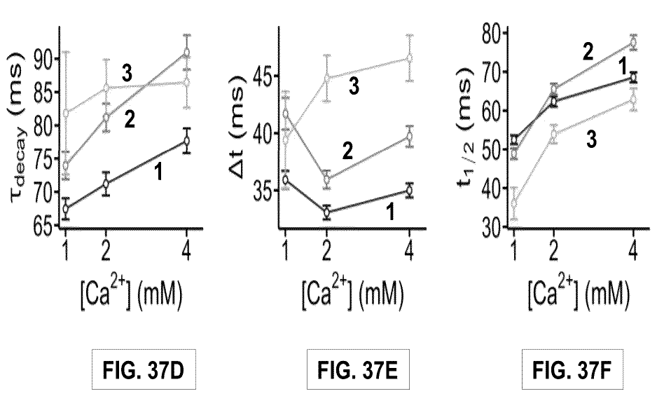

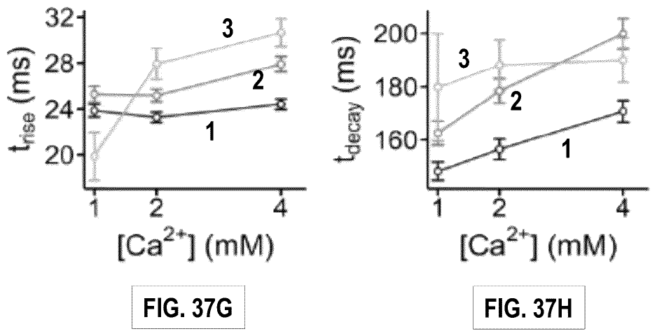

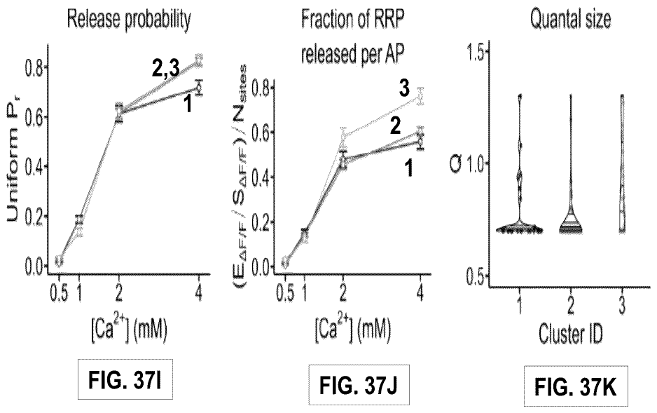

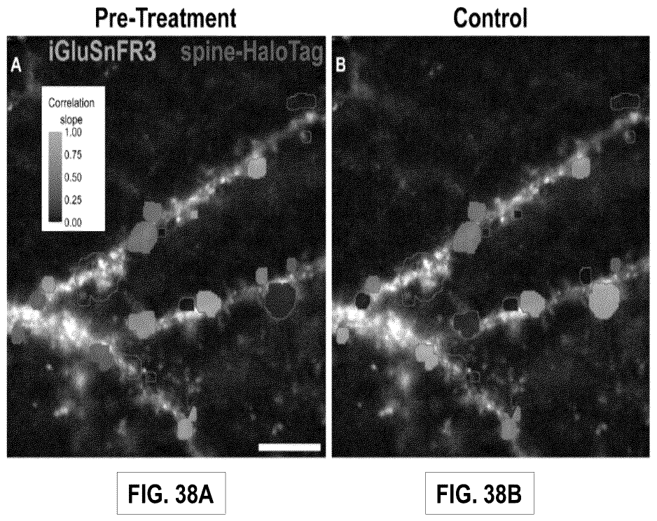

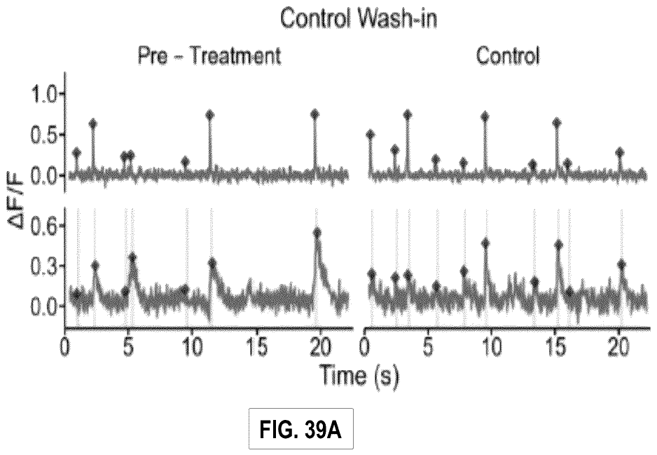

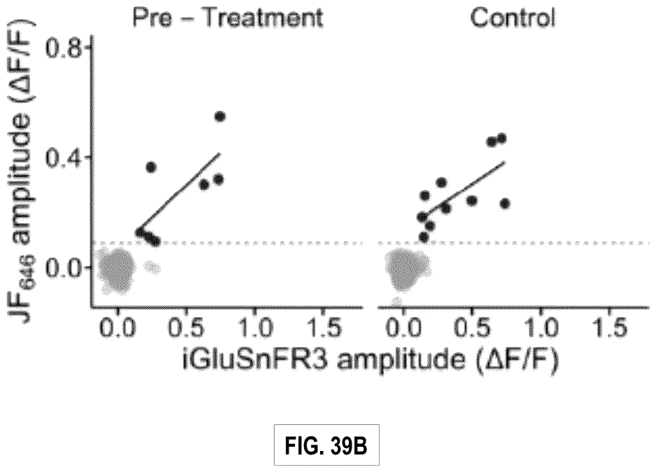

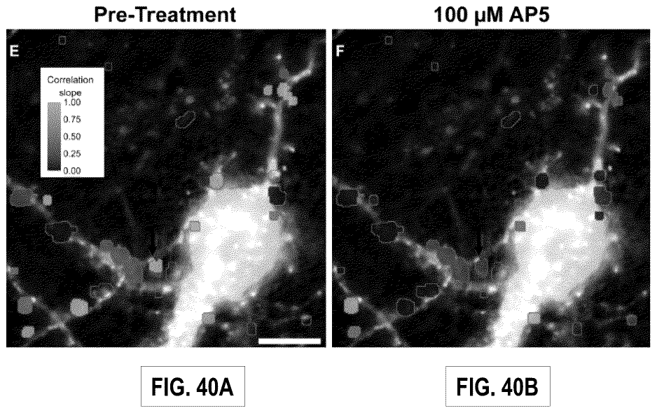

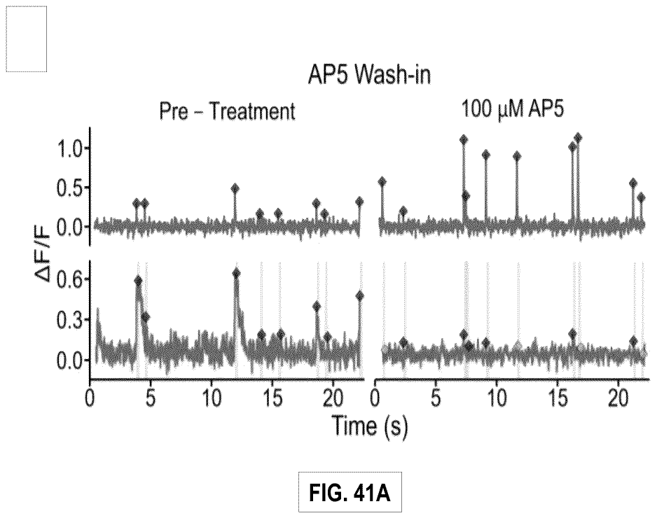

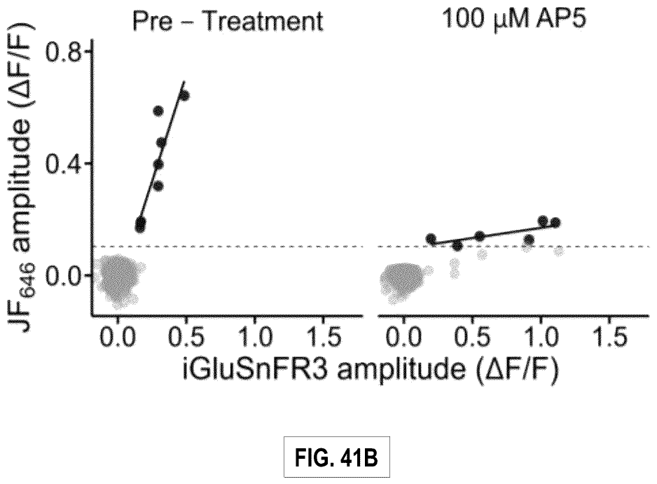

However, we also note these boutons possessed faster iGluSnFR3 kinetics (smaller Δt, tᵣᵢₛₑ, τ_{decay}) with lower variance in their response on all metrics relative to Class 2 and 3 boutons. FIG. 36 plots the average iGluSnFR3 response in response to stimulus across [Ca⁺⁺] bath and cluster ID. FIG. 37A through FIG. 37K are plots showing additional functional properties of the three bouton classes. FIG. 37A, coefficient of variation (CV) of peak ΔF/F vs. [Ca⁺⁺] bath; FIG. 37B, CV of τ_{decay} vs. [Ca⁺⁺] bath; FIG. 37C, CV of Δt vs. [Ca⁺⁺] bath; FIG. 37D, τ_{decay} vs. [Ca⁺⁺] bath; FIG. 37E, Δt vs. [Ca⁺⁺] bath; FIG. 37F, t½ vs. [Ca⁺⁺] bath; FIG. 37G, tᵣᵢₛₑ (10-90%) vs. [Ca⁺⁺] bath; FIG. 37H, τ_{decay} (90-10%) vs. [Ca⁺⁺] bath; FIG. 37I, uniform release probability, Pr vs. [Ca⁺⁺] bath; FIG. 37J, RRP fraction released per AP (EΔF/F/SΔF/F)/Nsites vs. [Ca⁺⁺] bath;

FIG. 37K, binomial model Q vs. cluster ID. Interestingly, the calculated Pᵣ of SV release sites was very similar for all boutons in the dataset regardless of cluster ID. Not wishing to be bound by theory, it is possible that while release site organization relative to Ca⁺⁺ channel clusters and/or Ca⁺⁺ influx during the AP may be similar across bouton classes, the release sites at Class 1 boutons are more functionally mature, driving SV fusion in a faster and more synchronized fashion following an AP.

A key advantage of optical physiology is that functional heterogeneity is spatially resolved. To visualize how bouton physiology varies along the axonal arbor, we mapped cluster IDs to their originating axonal arbors. FIG. 19 is an example imaging area 192 with each bouton separated by cluster ID. To aid visual comparison across boutons, the script generates a “Bouton Report” for each bouton that summarizes key features.

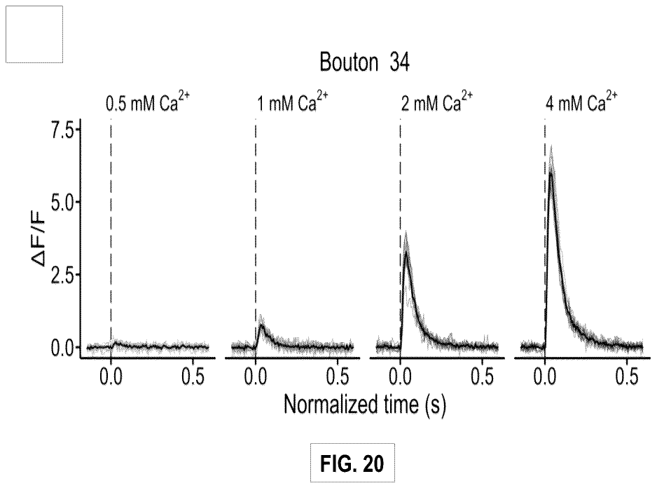

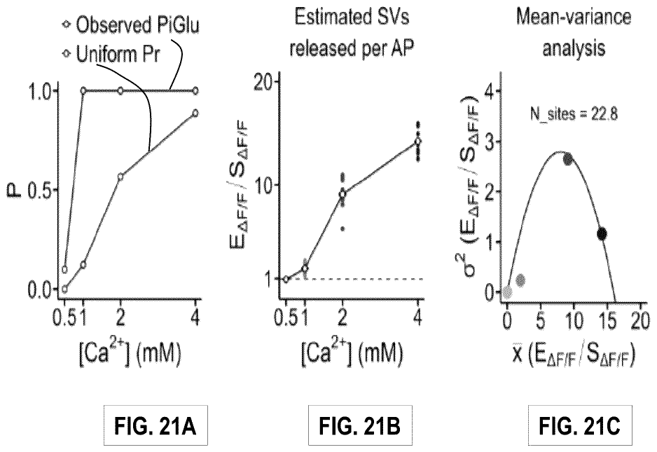

FIG. 20 through FIG. 21C show an example of a targeted functional analysis of Bouton 34, which includes the iGluSnFR3 response for all 40 single-stimulus trials. FIG. 20 is a set of graphs showing the averaged iGluSnFR3 waveform of single action potential (AP) trials for bouton 34. FIG. 21A presents the observed probability, PiGlu and the calculated uniform Pr vs. [Ca⁺⁺]_{bath}. This compares of P_{iGlu} with the observed uniform Pᵣ from MVA. FIG. 21B presents the E_{ΔF/F}/S_{ΔF/F} curve for Bouton 34 vs. [Ca2+]_{bath} which shows the estimated SVs released per AP for each trial. FIG. 21C presents a scatterplot of variance (σ2) of E_{ΔF/F}/S_{ΔF/F} vs. mean (\tilde{x}) E_{ΔF/F}/S_{ΔF/F} for Bouton 34. The binomial fit of the mean-variance curve fits the data. The colored dots indicate the measured σ2 and x at each [Ca⁺⁺] bath, while the black curve indicates the binomial fit.

Together, these tools enable scalable, functional profiling of individual boutons with high information content, which allows development of single synapse “function-omics”.

### Example 6: Paired Stimuli Reveal Heterogeneous Short-Term Plasticity Across Single Boutons

The presynaptic terminal can contribute to synapse response via short-term plasticity, in which pairs of stimuli delivered in short succession can drive increased (facilitation) or decreased (depression) neurotransmitter release on the second pulse. Encouraged by the heterogeneity we observed in our single stimulus experiments, we next asked whether our high-throughput approach could capture similar diversity in the short-term plasticity at single boutons. We imaged axonal arbors co-expressing iGluSnFR3 and Synaptophysin-mRuby, and recorded iGluSnFR3 activity in response to a battery of stimulus paradigms, including a single stimulus (test pulse) and paired stimuli separated by inter-stimulus intervals (ISIs) of 60, 75, 100, 150, or 500 ms. For each region, each of these stimulus protocols was administered in triplicate (20 seconds between trials). We began each imaging session with [Ca⁺⁺]_{bath}=0.5 mM, administering the protocol sequence before washing in buffer containing 1 mM or 2 mM Ca⁺⁺. We collected iGluSnFR3 activity from 149 putative boutons at 6 neurons. At [Ca⁺⁺]_{bath}=0.5 mM, more than 75% of boutons failed to respond at all ISIs, so the analysis below is presented with only [Ca⁺⁺]_{bath}=1 and 2 mM.

FIG. 22 shows the averaged iGluSnFR3 response for each stimulus protocol administered in this experiment across [Ca⁺⁺]bath=1, 2 mM. In this study, the goal was to directly measure the paired-pulse ratio (PPR) at single boutons, capturing heterogeneous short-term plasticity behavior along the axonal arbor. However, the kinetics of iGluSnFR3 resulted in a convolution of the responses to the 1st and 2nd stimuli at ISIs≤150 ms, obscuring the true amplitude of the second stimulus response. Dashed vertical lines indicate the stimulus.

To circumvent this, we subtracted the average response to a single stimulus at each bouton from the responses for each paired pulse trial, allowing measurement of the 2nd stimulus response at all ISIs (FIG. 23), and subsequently, PPRs for each bouton for each stimulus protocol. FIG. 23 shows the effects of subtraction for determining paired-pulse ratios (PPRs) at individual

boutons. Left: the amplitude of Pulse1 was the maximum ΔF/F observed within the first stimulus epoch in the unmodified, Original Trace for each trial. Middle: for each bouton, we determined the average iGluSnFR3 response to a single stimulus (Test Pulse) from three trials. Right: the amplitude of Pulse2 was the maximum ΔF/F observed within the second stimulus epoch after subtracting the Test Pulse from the Original Trace for each trial.

FIG. 24 is a set of violin plots showing the value of PPR as a function of [Ca⁺⁺] bath, interstimulus interval (ISI). Each shows the measured PPRs at all ISIs and [Ca⁺⁺]_{bath}. At [Ca⁺⁺]_{bath}=2 mM, most boutons depressed, consistent with an elevated initial release probability that depletes release sites following the 1st stimulus and failure to dock and prime new SVs prior to the second stimulus. A dot indicates the mean value of PPR for each bouton. The dashed line indicates PPR=1, the threshold between facilitation (PPR>1) and depression (PPR<1).

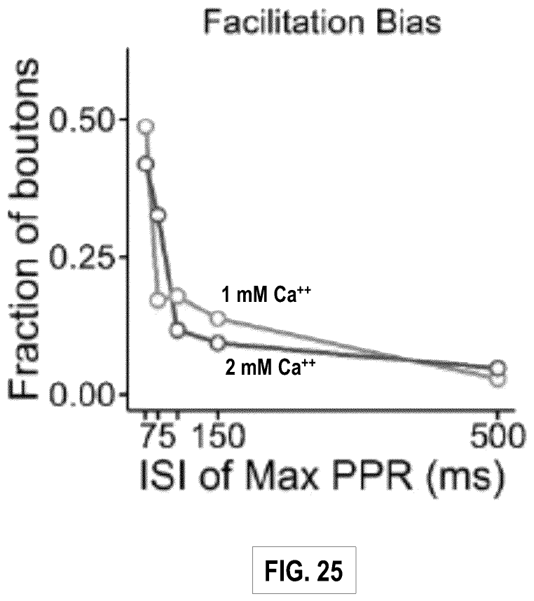

By contrast, at [Ca⁺⁺]bath=1 mM we observed that boutons could facilitate, depress, or exhibit no change in their response at the second stimulus for all ISIs. To ask whether boutons exhibited heterogeneous short-term plasticity dynamics, we measured which ISI generated the largest PPR at each bouton. We observed that 49% of boutons exhibited a “facilitation preference” for our shortest measured ISI at 60 ms. FIG. 25 shows categorized boutons according to which ISI produced the maximum value of PPR for boutons, termed the “Facilitation Bias.” Intriguingly, the facilitation preference for the second largest category of boutons (18%) was at ISI=100 ms, suggesting boutons possess diverse time constants which describe their short-term plasticity dynamics. Of the facilitating boutons (PPR≥1.1), 48.6% facilitated most strongly at ISI=60 ms and [Ca⁺⁺]_{bath}=1 mM.

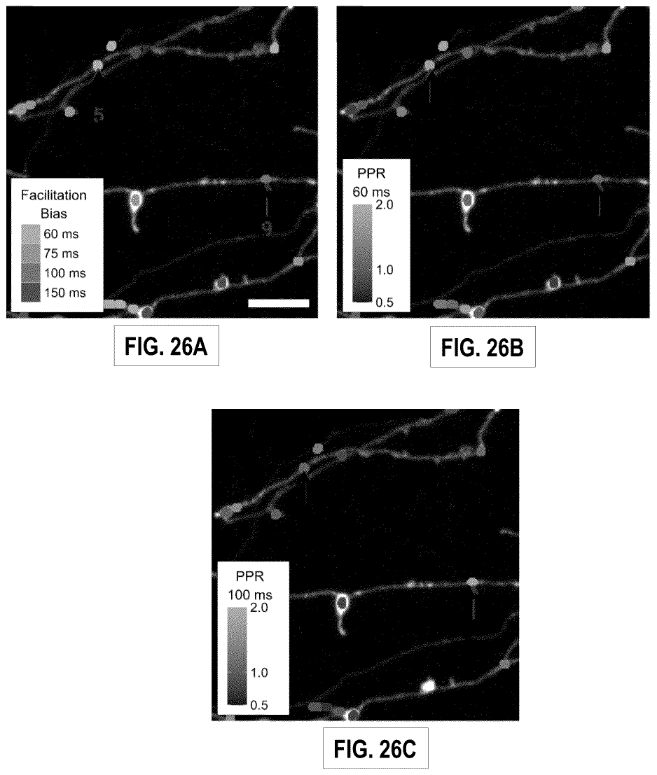

To better understand the heterogeneity of plasticity across the axonal arbor, we mapped the response to paired stimuli. FIG. 26A, FIG. 26B, and FIG. 26C show measures of plasticity mapped to an example of an imaging area 192 for [Ca⁺⁺]_{bath}=1 mM. Facilitation preference is mapped in FIG. 26A, and PPRs for selected ISIs (60, 100 ms) are mapped in FIG. 26B and FIG. 26C, respectively. Boutons in this imaging area were liable to preferentially facilitate at any of the ISIs tested except for 500 m.

We selected two boutons with divergent properties for targeted analysis: FIG. 27A shows individual trial responses by [Ca⁺⁺] bath with the average response for Bouton 5. FIG. 27B shows PPR curves across ISIs for 1 mM and 2 mM Ca⁺⁺ for Bouton 5. Bouton 5 is also depicted at upper left in FIG. 26A. FIG. 28A shows individual trial responses by [Ca⁺⁺]_{bath} with the averaged response for Bouton 9. FIG. 28B shows PPR curves across ISIs for 1 mM and 2 mM Ca⁺⁺ for Bouton 9. Bouton 9 is also depicted at middle right in FIG. 26A.

Strikingly, while Boutons 5 and 9 possessed similar glutamate release magnitudes in FIG. 27A and FIG. 28A, they diverge strongly in the presentation of their PPR curves in FIG. 27B and FIG. 28B). This suggests that while they may operate using similar numbers of release sites, distinctive mechanisms mediate their facilitation behavior. The functional imaging toolkit demonstrated here could be combined with post-hoc immunocytochemistry or super-resolution imaging of synaptic proteins to define how protein expression and organization at the synapse drive diverse short-term plasticity behavior across single neurons.

In this imaging area of FIG. 26A, no boutons facilitated most strongly at ISI=500 ms. Scale bar=5 μm. FIG. 26B and FIG. 26C show PPR mapped to the same axonal arbor as in FIG. 26A. Lighter colors indicate facilitation, while darker colors indicate depression. FIG. 26B shows PPRs for ISI=60 ms, [Ca⁺⁺] bath=1 mM. FIG. 26C shows PPRs for ISI=100 mseconds, [Ca⁺⁺] bath=1 mM. In FIG. 26A through FIG. 5G-26C, red arrows indicate boutons targeted for functional analysis.

### Example 7: Simultaneous Imaging of iGluSnFR3 and a Far-Red Ca⁺⁺ Reporter Enables Direct, all-Optical Measurement of NMDAR-Mediated Synaptic Transmission at Single Dendritic Spines

In standard postsynaptic patch-clamp recordings, measures of presynaptic function are indirect and postsynaptic measures lack spatial specificity. Dual optical assays of pre- and postsynaptic function help to untangle the basic functional properties of single synapses. To address this need, we focused on detecting activation of NMDARs, since dysregulated NMDAR-mediated synaptic transmission is implicated in plasticity and multiple neuropsychiatric illnesses. We paired iGluSnFR3 with a red-shifted Ca⁺⁺ reporter targeted to the dendritic spine to allow us to directly resolve NMDAR-mediated synaptic transmission at individual dendritic spines with simultaneous presynaptic readout. Previous work introduced spine-jRGECO1a, a fusion construct that expresses jRGECO1a in actin-rich compartments of the cell like the dendritic spine. However, 488 nm light causes jRGECO1a to photoswitch, dramatically reducing SNR and rendering spine-jRGECO1a incompatible with iGluSnFR3 in our hands. To avoid this, we replaced the coding sequence for jRGECO1a with that of HaloTag, which could be labeled with a far-red chemigenetic Ca⁺⁺ sensor, JF₆₄₆-BAPTA-HaloTagLigand-AM (hereafter, JF₆₄₆-BAPTA). Co-expression of iGluSnFR3 with spine-HaloTag thus enabled simultaneous access to dendritic spine glutamate and Ca⁺⁺ dynamics.

FIG. 29 is a set of photographs showing a example of an imaging region 192 with a neuron expressing both iGluSnFR3 (1^{st} column) and spine-HaloTag™ dyed with JF646-BAPTA-HTL-AM (2^{nd} column) and merged (3^{rd} column). Spine-HaloTag accumulates in actin-rich compartments of the cell, like the dendritic spine head. FIG. 30A presents a ΔF/F vs. time trace with spontaneous activity collected from iGluSnFR3 and JF646-BAPTA-AM from a single dendritic spine head. In the iGluSnFR3 trace, our peak finding algorithm identified iGluSnFR3 signals and their peak maxima (diamonds). In the JF646-BAPTA-AM trace, grey shaded vertical bars indicate times around iGluSnFR3 signals. To identify the peaks of JF646-BAPTA signals, we searched for local maxima in these grey shaded regions (diamonds). Insets show the corresponding dendritic spine at rest and during active synaptic transmission.

To observe NMDAR activation by spontaneously released glutamate, we imaged hippocampal neurons co-expressing iGluSnFR3 and spine-HaloTag in buffer with 4 mM Ca⁺⁺, 0 mM Mg⁺⁺, and 1 μM TTX. We segmented these recordings by iGluSnFR3 activity, observing iGluSnFR3 transients localized to dendritic spines that were often (but not always) followed by a JF₆₄₆-BAPTA transient. To analyze these, we used the iGluSnFR3 peaks detected by our algorithm to guide our identification of correlated JF₆₄₆-BAPTA peaks. We considered these event pairs to be putative synaptic transmission events if the JF₆₄₆-BAPTA amplitude exceeded a threshold of +3.5σ (σ is the standard deviation of the JF₆₄₆-BAPTA baseline. FIG. 30B is a scatterplot of JF646 peak amplitude vs. the iGluSnFR3 peak amplitude for the spine in FIG. 30A

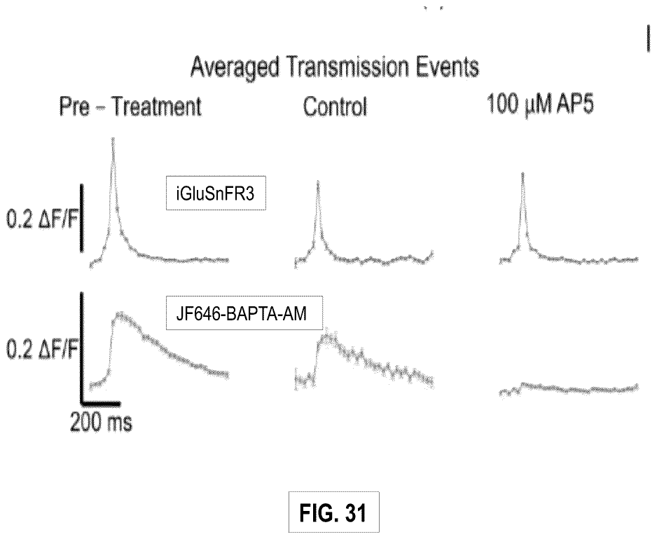

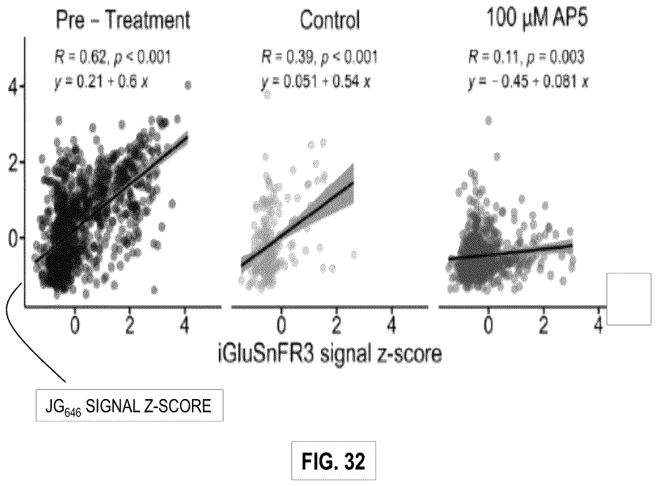

To determine whether these JF₆₄₆-BAPTA signals represent NMDAR activation by glutamate, we imaged neurons before and after perfusion of an NMDAR antagonist, AP5, or a control solution. For most spines, the amplitude correlation between iGluSnFR3 and JF₆₄₆-BAPTA signals remained the same following perfusion of control solution, but perfusion of 100 μM AP5 abolished JF₆₄₆-BAPTA signals without affecting iGluSnFR3. FIG. 31 shows the averaged waveforms of all collected transmission events from spines before and after exposure to a control solution or a solution containing 100 μM AP5 iGluSnFR3 (top) and JF646-BAPTA (bottom). FIG. 32 is a set of plots of population correlations between iGluSnFR3 and JF646-BAPTA signals from normalized transmission events by spine (z-score).

When we time-aligned transmission events and averaged them together, it became obvious that AP5 treatment abolished JF₆₄₆-BAPTA events, indicating that this Ca⁺⁺ activity originated from NMDAR activation. By contrast, the amplitude of iGluSnFR3 events after wash-in of either solution decreased similarly, presumably due to photobleaching.

The high steady-state affinity of NMDARs has often been taken to indicate that even the glutamate released from single vesicles will be sufficient to maximize activation of all NMDARs in a synapse. On the contrary, we observed many spines which exhibited a dose-dependent behavior similar to that seen in FIG. 30A and FIG. 30B, suggesting large glutamate transients activate greater numbers of NMDARs.

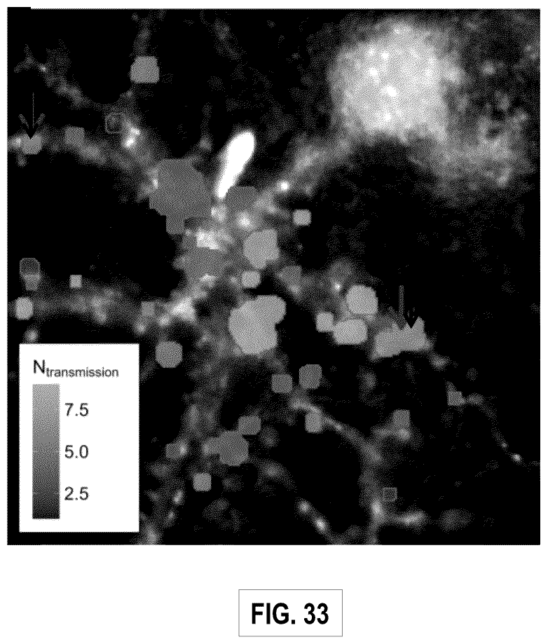

To analyze this, we normalized for spine-to-spine variation in iGluSnFR3 and JF₆₄₆-BAPTA by converting each spine's distribution of amplitudes to z-scores. FIG. 33 shows an example of an imaging area with dendritic spine ROIs shaded according to the recorded number of transmission events (Nₜᵣₐₙₛₘᵢₛₛᵢₒₙ). we collected all transmission events from spines before and after exposure to a control solution or a solution containing 100 μM AP5.

FIG. 32 shows from the averaged waveforms that AP5 treatment abolished JF646-BAPTA signals, but not iGluSnFR3 signals, indicating that JF646-BAPTA signals represent NMDAR-mediated Ca2+ flux. In FIG. 31 and FIG. 32, we normalized transmission events by spine (z-score) and plotted the population correlations between iGluSnFR3 and JF646-BAPTA signals. The strong positive correlation in the Pre-Treatment condition suggests that the amount of Ca⁺⁺ fluxed by NMDARs is dependent on the local concentration of glutamate during NMDAR activation. The slope of the correlation is largely intact following treatment of spines with a control solution, while treatment with 100 μM AP5 abolished the correlation. The Pearson correlation coefficient (R) and statistical significance are indicated on each subplot.

Similar to the single spine behavior seen in FIG. 30A and FIG. 30B, at the population level we observed a strong positive correlation between JF₆₄₆-BAPTA and iGluSnFR3 amplitudes before and after perfusion with a control solution. By contrast, treatment with AP5 abolished the amplitude correlation. FIG. 30B is a scatterplot of JF646 peak amplitude vs. the iGluSnFR3 peak amplitude for the spine shown in FIG. 30A. Our peak finding algorithm identified putative baseline indices as part of its baseline adjustment routine. For each dendritic spine, we calculated the standard deviation (a) of the baseline noise. We set a threshold of 3.5σ, and JF646-BAPTA signals above this threshold were considered putative synaptic transmission events.

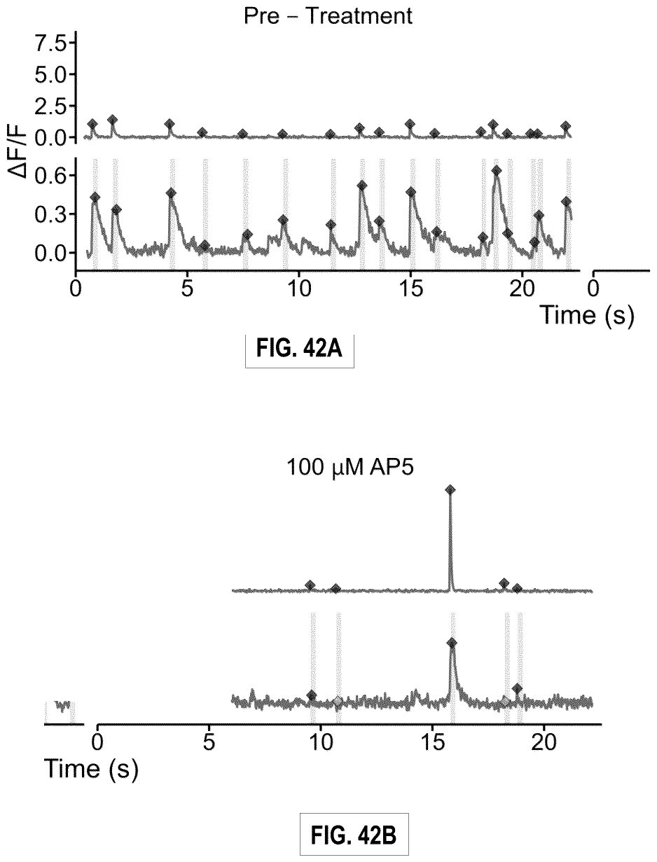

Remarkably, rare spines exhibited NMDAR activation following large iGluSnFR3 transients even after AP5 treatment. FIG. 42A and FIG. 42B show correlated iGluSnFR3 (top) and JF646 (bottom) activity before and after wash-in of 100 μM AP5. This suggests that glutamate can transiently out-compete AP5 to trigger NMDAR-mediated Ca⁺⁺ flux when the glutamate concentration in the synaptic cleft is sufficiently large. Note that this is the same spine as displayed in FIG. 30A. The y-axis of the iGluSnFR3 plot has been rescaled to accommodate the extremely large spontaneous iGluSnFR3 transient observed after 100 μM AP5 treatment. The data suggest that when the glutamate concentration in the cleft is sufficiently large, it can briefly out-compete AP5 to activate NMDARs.

Consistent with this, classic electrophysiology experiments examining NMDAR activation kinetics in outside-out patches from rat hippocampal neurons showed that application of 100 μM AP5 followed by a 100 ms application of 100 μM glutamate resulted in rapid and reliable NMDAR activation. Since our fluorescent reporter approach can access NMDAR-mediated synaptic transmission in situ at single dendritic spines, we anticipate this enables new lines of inquiry into the biophysics of NMDAR activation at the synapse during relevant neuronal activity patterns.

We also mapped the number of transmission events (Nₜᵣₐₙₛₘᵢₛₛᵢₒₙ) at an example of an imaging area 192 to appraise the heterogeneous activity across the dendritic arbor. FIG. 33 shows an example of an imaging area with dendritic spine ROIs shaded according to the recorded number of transmission events (Nₜᵣₐₙₛₘᵢₛₛᵢₒₙ). Nₜᵣₐₙₛₘᵢₛₛᵢₒₙ for this imaging area varied from 1 event to >10 events over the 30 seconds imaging epoch.

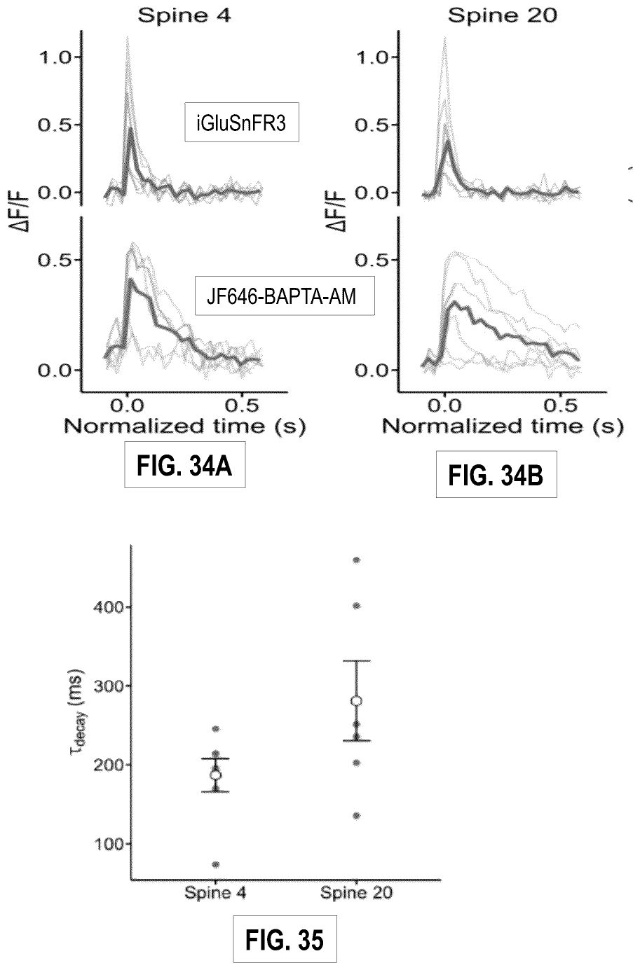

Two spines with similar activity profiles were selected for comparative analysis, indicated by the arrows. FIG. 34A and FIG. 34B show time-aligned iGluSnFR3 and JF646-BAPTA signals for each transmission event for Spine 4 and Spine 20. Average waveform is indicated by the bolded curve. The figures show distinct decay kinetics for JF₆₄₆-BAPTA events between Spine 4 and Spine 20, while the overall magnitude of JF₆₄₆-BAPTA transients were similar. FIG. 35 shows the distribution of τ_{decay} vs. spine identity. JF646-BAPTA signals at Spine 4 and Spine 20 possess distinct decay kinetics. The observation of kinetic differences between spines is consistent with distinct expression of NMDAR subtypes. These single spine recordings therefore are expected to be paired with super-resolution imaging of synaptic NMDARs. Such an approach can resolve open questions about how synaptic function is modified by the observed preference of NMDARs for certain glutamate release sites.

### Example 8: Boutons Separated by about 1.8 m Exhibit Divergent iGluSnFR3 Behavior

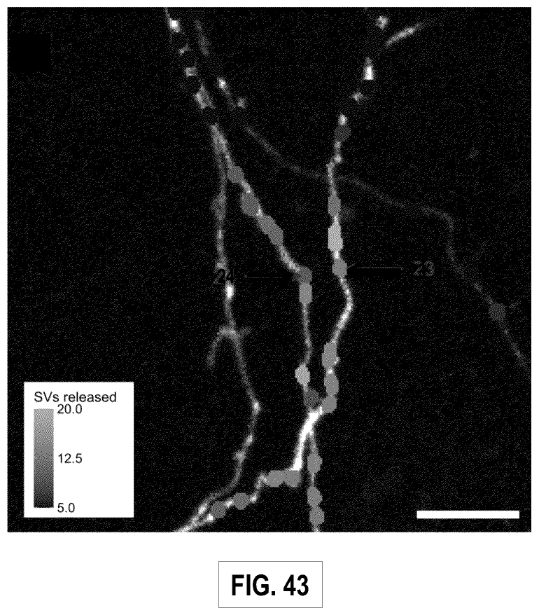

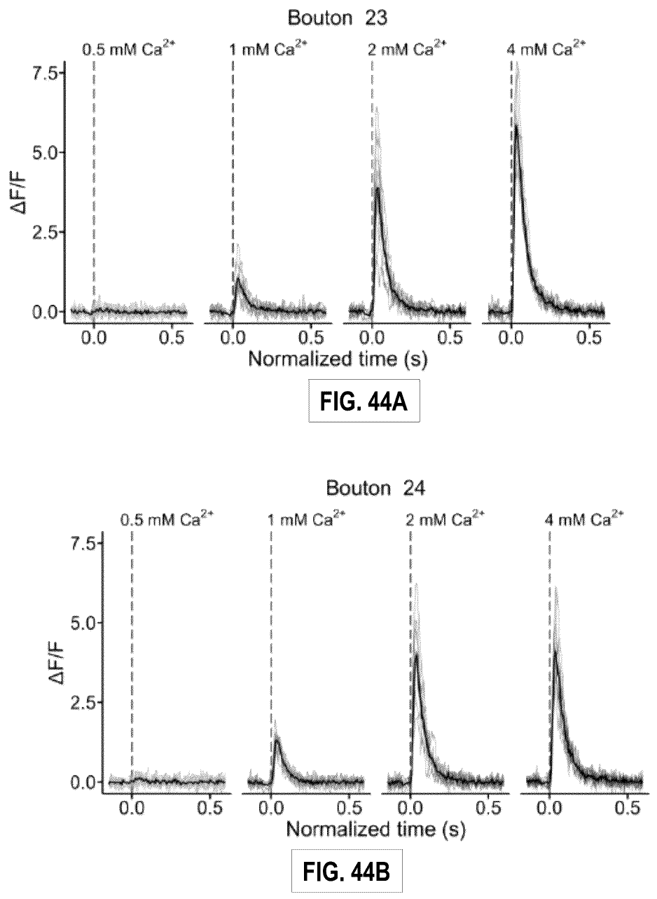

FIG. 43 shows the imaging region from FIG. 19, shaded according to number of SVs released at 4 mM Ca⁺⁺. Boutons 23 and 24 were selected for further analysis. FIG. 44A and FIG. 44B show the iGluSnFR3 responses at all [Ca⁺⁺]_{bath} tested. Though the boutons are only separated by 1.8 μm on separate branches of the same axon, they exhibit divergent iGluSnFR3 behavior.

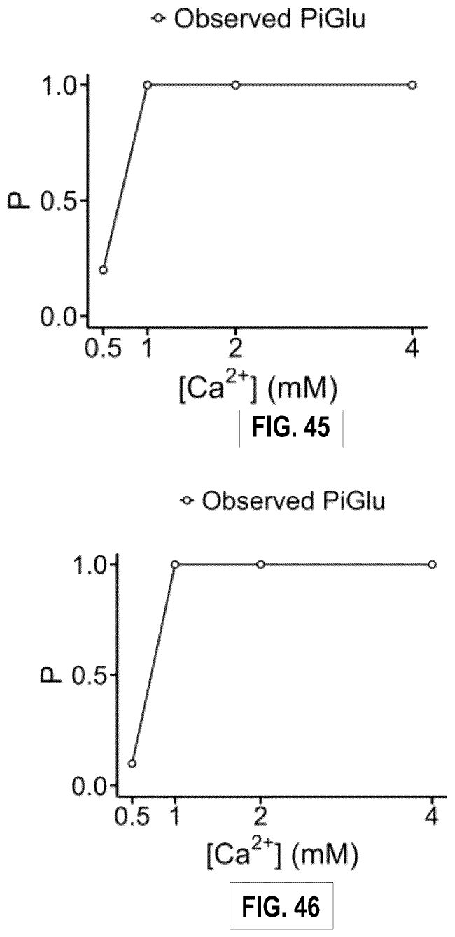

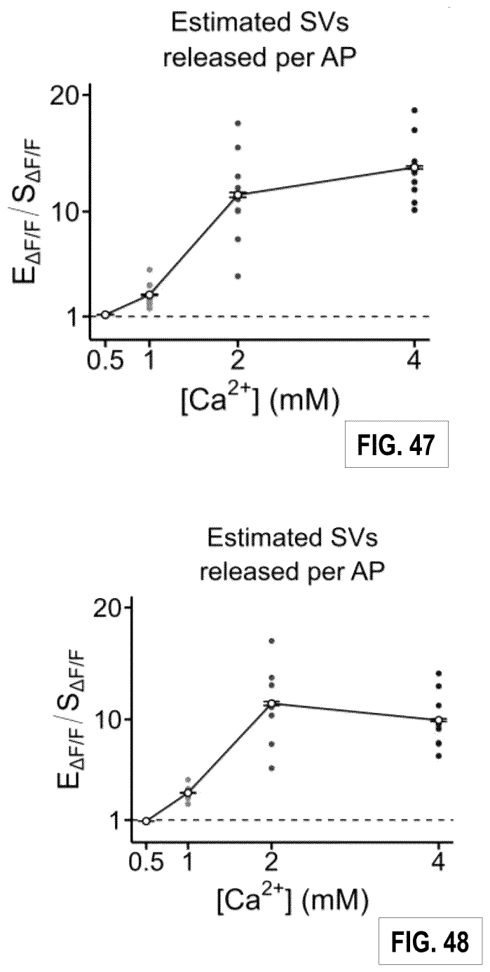

FIG. 45 and FIG. 46 show divergent iGluSnFR3 behavior for P_{iGlu} vs. [Ca⁺⁺]_{bath} at 0.5 mM. In FIG. 45, the synapse is active 30% of the time, while in FIG. 46, the synapse is active 10% of the time. FIG. 47 and FIG. 48 show divergent iGluSnFR3 behavior for estimated SVs released per AP (E_{ΔF/F}/S_{ΔF/F}) vs. [Ca2+]_{bath} Note that Bouton 24 appears to exhaust at 4 mM Ca⁺⁺.

### Example 9: Additional Functional Properties of the Three Bouton Classes Suggest Differences in the SV Release Machinery

FIG. 36 plots the average iGluSnFR3 response to stimulus across [Ca⁺⁺]_{bath} and cluster ID. FIG. 37A through FIG. 37K are plots showing additional functional properties of the three bouton classes. FIG. 37A, coefficient of variation (CV) of peak ΔF/F vs. [Ca⁺⁺]_{bath}; FIG. 37B, CV of τ_{decay} vs. [Ca⁺⁺]_{bath}; FIG. 37C, CV of Δt vs. [Ca⁺⁺]_{bath}; FIG. 37D, τ_{decay} vs. [Ca⁺⁺]_{bath}; FIG. 37E, Δt vs. [Ca⁺⁺]_{bath}; FIG. 37F, t½ vs. [Ca⁺⁺]_{bath}; FIG. 37G, trise (10-90%) vs. [Ca⁺⁺]_{bath}; FIG. 37H, τ_{decay} (90-10%) vs. [Ca⁺⁺]_{bath}; FIG. 37I, uniform release probability, Pr vs. [Ca⁺⁺]_{bath}; FIG. 37J, RRP fraction released per AP (E_{ΔF/F}/S_{ΔF/F})/Nsites vs. [Ca⁺⁺]_{bath}; FIG. 37K, binomial model Q vs. cluster ID, For the plots presented in FIG. 37A through FIG. 37F, 0.5 mM Ca2+ has been omitted due to the sparse data in clusters 2 and 3.

### Definitions

Unless defined otherwise, all technical and scientific terms used herein have the same meaning as commonly understood by one of ordinary skill in the art. Although various methods and materials similar or equivalent to those described herein can be used in the practice or testing of the present invention, suitable methods and materials used are described below. However, the skilled artisan understands that the methods and materials used and described are examples and may not be the only ones suitable for use in the invention. Moreover, as measurements are subject to inherent variability, any temperature, weight, volume, time interval, pH, salinity, molarity or molality, range, concentration, and any other measurements, quantities, or numerical expressions given herein are intended to be approximate and not exact or critical figures unless expressly stated to the contrary.

In the foregoing specification, the invention has been described with reference to specific embodiments thereof. It will, however, be evident that various modifications and changes may be made thereto without departing from the broader spirit and scope of the invention. The specification and drawings are, accordingly, to be regarded in an illustrative rather than a restrictive sense. Throughout this specification and the claims, unless the context requires otherwise, the word “comprise” and its variations, such as “comprises” and “comprising,” will be understood to imply the inclusion of a stated item, element or step or group of items, elements or steps but not the inclusion of any other item, element, step, or group of items, elements, or steps. Furthermore, the indefinite article “a” or “an” is meant to indicate one or more of the item, element, or step modified by the article.

As used herein, the term “about” means plus or minus 20 percent of the recited value so that, for example, “about 0.125” means 0.125±0.025, and “about 1.0” means 1.0±0.2. Notwithstanding that the numerical ranges and parameters setting for the broad scope of the invention are approximations, the numerical values set forth in specific non-limiting examples are reported as precisely as possible. Any numerical value, however, inherently contains certain errors necessarily resulting from the standard deviation found in their respective testing measurements at the time of this writing.

### 3. Computer Hardware Overview

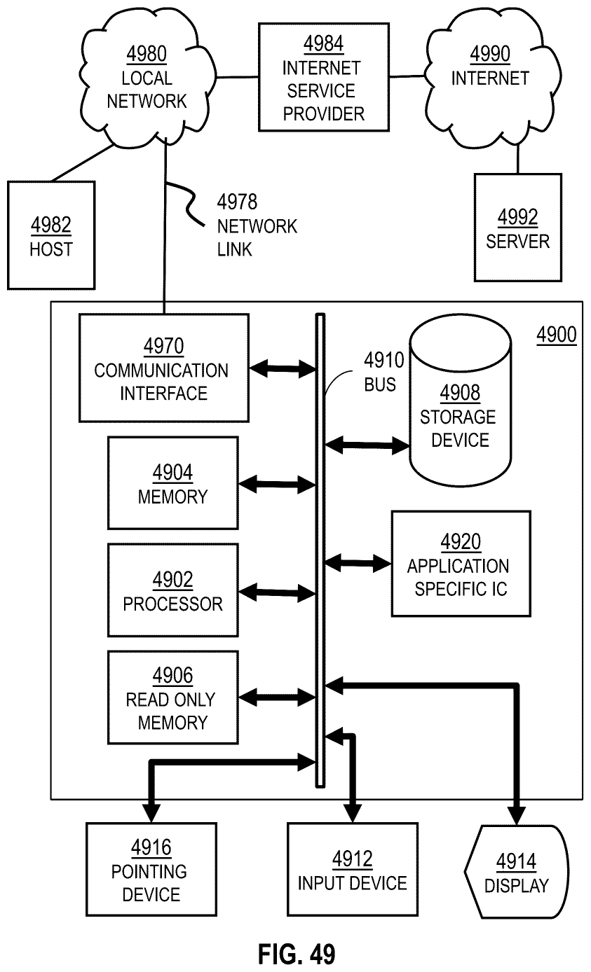

FIG. 49 is a block diagram that illustrates a computer system 4900 upon which an embodiment of the invention may be implemented. Computer system 4900 includes a communication mechanism such as a bus 4910 for passing information between other internal and external components of the computer system 4900. Information is represented as physical signals of a measurable phenomenon, typically electric voltages, but including, in other embodiments, such phenomena as magnetic, electromagnetic, pressure, chemical, molecular atomic and quantum interactions. For example, north and south magnetic fields, or a zero and non-zero electric voltage, represent two states (0, 1) of a binary digit (bit). Other phenomena can represent digits of a higher base. A superposition of multiple simultaneous quantum states before measurement represents a quantum bit (qubit). A sequence of one or more digits constitutes digital data that is used to represent a number or code for a character. In some embodiments, information called analog data is represented by a near continuum of measurable values within a particular range. Computer system 4900, or a portion thereof, constitutes a means for performing one or more steps of one or more methods described herein.

A sequence of binary digits constitutes digital data that is used to represent a number or code for a character. A bus 4910 includes many parallel conductors of information so that information is transferred quickly among devices coupled to the bus 4910. One or more processors 4902 for processing information are coupled with the bus 4910. A processor 4902 performs a set of operations on information. The set of operations include bringing information in from the bus 4910 and placing information on the bus 4910. The set of operations also typically include comparing two or more units of information, shifting positions of units of information, and combining two or more units of information, such as by addition or multiplication. A sequence of operations to be executed by the processor 4902 constitutes computer instructions.

Computer system 4900 also includes a memory 4904 coupled to bus 4910. The memory 4904, such as a random access memory (RAM) or other dynamic storage device, stores information including computer instructions. Dynamic memory allows information stored therein to be changed by the computer system 4900. RAM allows a unit of information stored at a location called a memory address to be stored and retrieved independently of information at neighboring addresses. The memory 4904 is also used by the processor 4902 to store temporary values during execution of computer instructions. The computer system 4900 also includes a read only memory (ROM) 4906 or other static storage device coupled to the bus 4910 for storing static information, including instructions, that is not changed by the computer system 4900. Also coupled to bus 4910 is a non-volatile (persistent) storage device 4908, such as a magnetic disk or optical disk, for storing information, including instructions, that persists even when the computer system 4900 is turned off or otherwise loses power.

Information, including instructions, is provided to the bus 4910 for use by the processor from an external input device 4912, such as a keyboard containing alphanumeric keys operated by a human user, or a sensor. A sensor detects conditions in its vicinity and transforms those detections into signals compatible with the signals used to represent information in computer system 4900. Other external devices coupled to bus 4910, used primarily for interacting with humans, include a display device 4914, such as a cathode ray tube (CRT) or a liquid crystal display (LCD), for presenting images, and a pointing device 4916, such as a mouse or a trackball or cursor direction keys, for controlling a position of a small cursor image presented on the display 4914 and issuing commands associated with graphical elements presented on the display 4914.

In the illustrated embodiment, special purpose hardware, such as an application specific integrated circuit (IC) 4920, is coupled to bus 4910. The special purpose hardware is configured to perform operations not performed by processor 4902 quickly enough for special purposes. Examples of application specific ICs include graphics accelerator cards for generating images for display 4914, cryptographic boards for encrypting and decrypting messages sent over a network, speech recognition, and interfaces to special external devices, such as robotic arms and medical scanning equipment that repeatedly perform some complex sequence of operations that are more efficiently implemented in hardware.

Computer system 4900 also includes one or more instances of a communications interface 4970 coupled to bus 4910. Communication interface 4970 provides a two-way communication coupling to a variety of external devices that operate with their own processors, such as printers, scanners and external disks. In general the coupling is with a network link 4978 that is connected to a local network 4980 to which a variety of external devices with their own processors are connected. For example, communication interface 4970 may be a parallel port or a serial port or a universal serial bus (USB) port on a personal computer. In some embodiments, communications interface 4970 is an integrated services digital network (ISDN) card or a digital subscriber line (DSL) card or a telephone modem that provides an information communication connection to a corresponding type of telephone line. In some embodiments, a communication interface 4970 is a cable modem that converts signals on bus 4910 into signals for a communication connection over a coaxial cable or into optical signals for a communication connection over a fiber optic cable. As another example, communications interface 4970 may be a local area network (LAN) card to provide a data communication connection to a compatible LAN, such as Ethernet. Wireless links may also be implemented. Carrier waves, such as acoustic waves and electromagnetic waves, including radio, optical and infrared waves travel through space without wires or cables. Signals include man-made variations in amplitude, frequency, phase, polarization or other physical properties of carrier waves. For wireless links, the communications interface 4970 sends and receives electrical, acoustic or electromagnetic signals, including infrared and optical signals, that carry information streams, such as digital data.

The term computer-readable medium is used herein to refer to any medium that participates in providing information to processor 4902, including instructions for execution. Such a medium may take many forms, including, but not limited to, non-volatile media, volatile media and transmission media. Non-volatile media include, for example, optical or magnetic disks, such as storage device 4908. Volatile media include, for example, dynamic memory 4904. Transmission media include, for example, coaxial cables, copper wire, fiber optic cables, and waves that travel through space without wires or cables, such as acoustic waves and electromagnetic waves, including radio, optical and infrared waves. The term computer-readable storage medium is used herein to refer to any medium that participates in providing information to processor 4902, except for transmission media.

Common forms of computer-readable media include, for example, a floppy disk, a flexible disk, a hard disk, a magnetic tape, or any other magnetic medium, a compact disk ROM (CD-ROM), a digital video disk (DVD) or any other optical medium, punch cards, paper tape, or any other physical medium with patterns of holes, a RAM, a programmable ROM (PROM), an erasable PROM (EPROM), a FLASH-EPROM, or any other memory chip or cartridge, a carrier wave, or any other medium from which a computer can read. The term non-transitory computer-readable storage medium is used herein to refer to any medium that participates in providing information to processor 4902, except for carrier waves and other signals.

Logic encoded in one or more tangible media includes one or both of processor instructions on a computer-readable storage media and special purpose hardware, such as ASIC 4920.

Network link 4978 typically provides information communication through one or more networks to other devices that use or process the information. For example, network link 4978 may provide a connection through local network 4980 to a host computer 4982 or to equipment 4984 operated by an Internet Service Provider (ISP). ISP equipment 4984 in turn provides data communication services through the public, world-wide packet-switching communication network of networks now commonly referred to as the Internet 4990. A computer called a server 4992 connected to the Internet provides a service in response to information received over the Internet. For example, server 4992 provides information representing video data for presentation at display 4914.

The invention is related to the use of computer system 4900 for implementing the techniques described herein. According to one embodiment of the invention, those techniques are performed by computer system 4900 in response to processor 4902 executing one or more sequences of one or more instructions contained in memory 4904. Such instructions, also called software and program code, may be read into memory 4904 from another computer-readable medium such as storage device 4908. Execution of the sequences of instructions contained in memory 4904 causes processor 4902 to perform the method steps described herein. In alternative embodiments, hardware, such as application specific integrated circuit 4920, may be used in place of or in combination with software to implement the invention. Thus, embodiments of the invention are not limited to any specific combination of hardware and software.

The signals transmitted over network link 4978 and other networks through communications interface 4970, carry information to and from computer system 4900. Computer system 4900 can send and receive information, including program code, through the networks 4980, 4990 among others, through network link 4978 and communications interface 4970. In an example using the Internet 4990, a server 4992 transmits program code for a particular application, requested by a message sent from computer 4900, through Internet 4990, ISP equipment 4984, local network 4980 and communications interface 4970. The received code may be executed by processor 4902 as it is received, or may be stored in storage device 4908 or other non-volatile storage for later execution, or both. In this manner, computer system 4900 may obtain application program code in the form of a signal on a carrier wave.

Various forms of computer readable media may be involved in carrying one or more sequence of instructions or data or both to processor 4902 for execution. For example, instructions and data may initially be carried on a magnetic disk of a remote computer such as host 4982. The remote computer loads the instructions and data into its dynamic memory and sends the instructions and data over a telephone line using a modem. A modem local to the computer system 4900 receives the instructions and data on a telephone line and uses an infra-red transmitter to convert the instructions and data to a signal on an infra-red a carrier wave serving as the network link 4978. An infrared detector serving as communications interface 4970 receives the instructions and data carried in the infrared signal and places information representing the instructions and data onto bus 4910. Bus 4910 carries the information to memory 4904 from which processor 4902 retrieves and executes the instructions using some of the data sent with the instructions. The instructions and data received in memory 4904 may optionally be stored on storage device 4908, either before or after execution by the processor 4902.

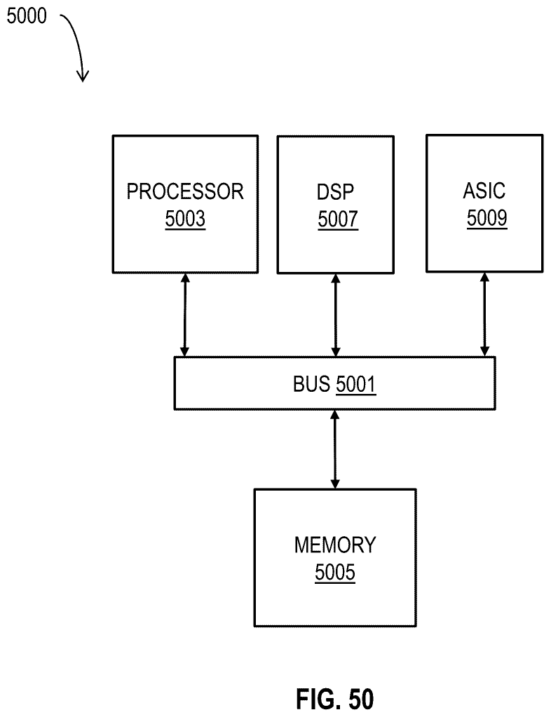

FIG. 50 illustrates a chip set 5000 upon which an embodiment of the invention may be implemented. Chip set 5000 is programmed to perform one or more steps of a method described herein and includes, for instance, the processor and memory components described with respect to FIG. 49 incorporated in one or more physical packages (e.g., chips). By way of example, a physical package includes an arrangement of one or more materials, components, and/or wires on a structural assembly (e.g., a baseboard) to provide one or more characteristics such as physical strength, conservation of size, and/or limitation of electrical interaction. It is contemplated that in certain embodiments the chip set can be implemented in a single chip. Chip set 5000, or a portion thereof, constitutes a means for performing one or more steps of a method described herein.

In one embodiment, the chip set 5000 includes a communication mechanism such as a bus 5001 for passing information among the components of the chip set 5000. A processor 5003 has connectivity to the bus 5001 to execute instructions and process information stored in, for example, a memory 5005. The processor 5003 may include one or more processing cores with each core configured to perform independently. A multi-core processor enables multiprocessing within a single physical package. Examples of a multi-core processor include two, four, eight, or greater numbers of processing cores. Alternatively, or in addition, the processor 5003 may include one or more microprocessors configured in tandem via the bus 5001 to enable independent execution of instructions, pipelining, and multithreading. The processor 5003 may also be accompanied with one or more specialized components to perform certain processing functions and tasks such as one or more digital signal processors (DSP) 5007, or one or more application-specific integrated circuits (ASIC) 5009. A DSP 5007 typically is configured to process real-world signals (e.g., sound) in real time independently of the processor 5003. Similarly, an ASIC 5009 can be configured to performed specialized functions not easily performed by a general purposed processor. Other specialized components to aid in performing the inventive functions described herein include one or more field programmable gate arrays (FPGA) (not shown), one or more controllers (not shown), or one or more other special-purpose computer chips.

The processor 5003 and accompanying components have connectivity to the memory 5005 via the bus 5001. The memory 5005 includes both dynamic memory (e.g., RAM, magnetic disk, writable optical disk, etc.) and static memory (e.g., ROM, CD-ROM, etc.) for storing executable instructions that when executed perform one or more steps of a method described herein. The memory 5005 also stores the data associated with or generated by the execution of one or more steps of the methods described herein.

### REFERENCES

All publications listed below and throughout the specification are hereby incorporated by reference in their entirety. Nothing herein is to be construed as an admission that the invention is not entitled to antedate such disclosure by virtue of prior invention.
1. Südhof, T C & Malenka, R C. “Understanding Synapses: Past, Present, and Future.” Neuron, 2018, 60, 469-476.
2. Abbott, L F, & Regehr, W G. “Synaptic computation.” Nature, 2004, 431, 796-803.
3. Branco, T, & Staras, K. “The probability of neurotransmitter release: Variability and feedback control at single synapses.” Nature Reviews Neuroscience, 2009, 10, 373-383.
4. Regehr, W G. “Short-term presynaptic plasticity.” Cold Spring Harbor Perspectives in Biology, 2012, 4, 1-19.
5. O'Rourke, N A, Weiler, N C, Micheva, K D, & Smith, S J. “Deep molecular diversity of mammalian synapses: Why it matters and how to measure it.” Nature Reviews Neuroscience, 2012, 13, 365-379.
6. Grant, S G N, & Fransén, E. “The Synapse Diversity Dilemma: Molecular Heterogeneity Confounds Studies of Synapse Function.” Front. Synaptic Neurosci., 2020, 12, 1-8.
7. Xu, H, Oses-Prieto, J A, Khvotchev, M, Jain, S, Liang, J, Burlingame, A, & Edwards, R H. “Adaptor protein AP-3 produces synaptic vesicles that release at high frequency by recruiting phospholipid flippase ATP8A1.” Nature Neuroscience, 2023, 26, 1685-1700.
8. Jackman, S L, Turecek, J, Belinsky, J E, & Regehr, W G. “The calcium sensor synaptotagmin 7 is required for synaptic facilitation.” Nature, 2016, 529, 88-91.
9. Paoletti, P, Bellone, C, & Zhou, Q. “NMDA receptor subunit diversity: impact on receptor properties, synaptic plasticity and disease.” Nature Reviews Neuroscience, 2013, 14, 383-400.
10. Carter, A G, & Sabatini, B L. “State-dependent calcium signaling in dendritic spines of striatal medium spiny neurons.” Neuron, 2004, 44, 483-493.
11. Lin, M Z, & Schnitzer, M J. “Genetically encoded indicators of neuronal activity.” Nature Neuroscience, 2016, 19, 1142-1153.
12. Hao, Y, & Plested, A J R. “Seeing glutamate at central synapses.” Journal of Neuroscience Methods, 2022, 375, 1-13.
13. Balaji, J, & Ryan, T A. “Single-vesicle imaging reveals that synaptic vesicle exocytosis and endocytosis are coupled by a single stochastic mode.” PNAS, 2007, 104, 20576-20581.
14. Sakamoto, H, Ariyoshi, T, Kimpara, N, Sugao, K, Taiko, I, Takikawa, K, Asanuma, D, Namiki, S, & Hirose, K. “Synaptic weight set by Munc13-1 supramolecular assemblies.” Nature Neuroscience, 2018, 21, 41-55.
15. Kleinfeld, D, Giovannucci, A, Friedrich, J, et al. “CaImAn an open source tool for scalable calcium imaging data analysis.” eLife, 8, 2019, 1-45.
16. Cai, C, Dong, C, Friedrich, J, Rozsa, M, Pnevmatikakis, E A, & Giovannucci, A. “FIOLA: an accelerated pipeline for fluorescence imaging online analysis.” Nature Methods, 2023, 20, 1417-1425.
17. Cai, C, Friedrich, J, Singh, A, Eybposh, M H, Pnevmatikakis, E A, Podgorski, K, & Giovannucci, A. “VoIPy: Automated and scalable analysis pipelines for voltage imaging datasets.” PLoS Computational Biology, 2021, 17, 1-27.
18. Aggarwal, A, et al. “Glutamate indicators with improved activation kinetics and localization for imaging synaptic transmission.” Nature Methods, 2023, 20, 925-934.
19. Durst, C D, Wiegert, J S, Schulze, C. et al. “Vesicular release probability sets the strength of individual Schaffer collateral synapses.” Nat Commun 2022, 13, 6126.
20. Helassa, N, Durst, C D, Coates, C, Kerruth, S, Arif, U, Schulze, C, Simon Wiegert, J, Geeves, M, Oertner, T G, & Török, K. “Ultrafast glutamate sensors resolve high-frequency release at Schaffer collateral synapses.” PNAS, 2018, 115, 5594-5599.
21. Courtney, N A, Briguglio, J S, Bradberry, M M, Greer, C, & Chapman, E R. “Excitatory and Inhibitory Neurons Utilize Different Ca2+ Sensors and Sources to Regulate Spontaneous Release.” Neuron, 2018, 98, 977-991.
22. Wang, Y, Wang, C, Ranefall, P, Broussard, G J, Wang, Y, Shi, G, Lyu, B, Wu, C T, Wang, Y, Tian, L, & Yu, G. “SynQuant: An automatic tool to quantify synapses from microscopy images.” Bioinformatics, 2020, 36, 1599-1606.
23. Mendonça, PRF, Tagliatti, E, Langley, H, Kotzadimitriou, D, Zamora-Chimal, C G, Timofeeva, Y, & Volynski, K E. “Aynchronous glutamate release is enhanced in low release efficacy synapses and dispersed across the active zone.” Nature Communications, 2022, 13, 1-17.
24. Taschenberger, H, Woehler, A, & Neher, E. “Superpriming of synaptic vesicles as a common basis for intersynapse variability and modulation of synaptic strength.” PNAS, 2016, 113, E4548-E4557.
25. Maschi, D, & Klyachko, V A. “Spatiotemporal dynamics of multi-vesicular release is determined by heterogeneity of release sites within central synapses.” eLife, 2020, 9, 1-20.
26. Tang, A H, Chen, H, Li, T P, Metzbower, S R, MacGillavry, H D, & Blanpied, T A. “A trans-synaptic nanocolumn aligns neurotransmitter release to receptors.” Nature, 2016, 536, 210-214.
27. Kaeser, P S, & Regehr, W G. “The readily releasable pool of synaptic vesicles.” Current Opinion in Neurobiology, 2017, 43, 63-70.
28. Clements, J D, & Silver, R A. “Unveiling synaptic plasticity: a new graphical and analytical approach.” Trends in Neurosciences, 2000, 23, 105-113.
29. Malagon, G, Miki, T, Llano, I, Neher, E, & Marty, A. “Counting vesicular release events reveals binomial release statistics at single glutamatergic synapses.” Journal of Neuroscience, 2016, 36, 4010-4025.
30. Glasgow, S D, McPhedrain, R, Madranges, J F, Kennedy, T E, & Ruthazer, E S. “Approaches and limitations in the investigation of synaptic transmission and plasticity.” Frontiers in Synaptic Neuroscience, 2019, 11, 1-16.
31. Sakamoto, H, Ariyoshi, T, Kimpara, N, Sugao, K, Taiko, I, Takikawa, K, Asanuma, D, Namiki, S, & Hirose, K. “Synaptic weight set by Munc13-1 supramolecular assemblies.” Nature Neuroscience, 2018, 21, 41-55.
32. Kusick, G F, Chin, M, Raychaudhuri, S, Lippmann, K, Adula, K P, Hujber, E J, Vu, T, Davis, M W, Jorgensen, E M, & Watanabe, S. “Synaptic vesicles transiently dock to refill release sites.” Nature Neuroscience, 2020, 23, 1329-1338.
33. McInnes, L, Healy, J, Saul, N & Großberger, L. “UMAP: uniform manifold approximation and projection.” J. Open Source Softw. 2018, 3, 861.
34. Volk, L, Chiu, S L, Sharma, K, & Huganir, R L. “Glutamate Synapses in Human Cognitive Disorders.” Annual Review of Neuroscience, 2015, 38, 127-149.
35. Fan, L Z, Nehme, R, Adam, Y, Jung, E S, Wu, H, Eggan, K, Arnold, D B, & Cohen, A E. “All-optical synaptic electrophysiology probes mechanism of ketamine-induced disinhibition.” Nature Methods, 2018, 15, 823-831.
36. Dana, H, et al. “Sensitive red protein calcium indicators for imaging neural activity.” eLife, 2016, 5, 1-24.
37. Los, G V, Encell, L P, Mcdougall, M G, Hartzell, D D, Karassina, N, Simpson, D, Mendez, J, Zimmerman, K, Otto, P, Vidugiris, G, Zhu, J, Darzins, A, Klaubert, D H, Bulleit, R F, & Wood, K V. “HaloTag: A Novel Protein Labeling Technology for Cell Imaging and Protein Analysis.” ACS Chemical Biology, 2008, 3, 373-382.
38. Deo, C, Sheu, S, Seo, J, Clapham, D E, & Lavis, L D. “Isomeric Tuning Yields Bright and Targetable Red Ca2+ Indicators.” JACS, 2019, 141, 13734-13738.
39. Metzbower, S R, Joo, Y, Benavides, D R, & Blanpied, T A. “Properties of Individual Hippocampal Synapses Influencing NMDA-Receptor Activation by Spontaneous Neurotransmission.” eNeuro, 2019, 6, 1-17.
40. Lester, R A J, Clements, J D, Westbrook, G L, & Jahr, C E. “Channel kinetics determine the time course of NMDA receptor-mediated synaptic currents.” Nature, 1990, 346, 565-567.
41. Niswender, C M & Conn, P J. “Metabotropic Glutamate Receptors: Physiology, Pharmacology, and Disease.” Annu Rev Pharmacol Toxicol. 2010, 50, 295-322.
42. Barri, A, Wiechert, M T, Jazayeri, M, & DiGregorio, D A. “Synaptic basis of a sub-second representation of time in a neural circuit model.” Nature Communications, 2022, 13, 1-18.
43. Chen, J J, Kaufmann, W A, Chen, C, Arai, I, Kim, O, Shigemoto, R, & Jonas, P. “Developmental transformation of Ca2+ channel-vesicle nanotopography at a central GABAergic synapse.” Neuron, 2024, 112, 755-771.
44. Bradberry, M M & Chapman, E R. “All-optical monitoring of excitation secretion coupling demonstrates that SV2A functions downstream of evoked Ca2+ entry.” J. Physiology, 2022, 600, 645-654.
45. Almada, P, Pereira, P M, Culley, S, Caillol, G, Boroni-Rueda, F, Dix, C L, Charras, G, Baum, B, Laine, R F, Leterrier, C, & Henriques, R. “Automating multimodal microscopy with NanoJ-Fluidics.” Nature Communications, 2019, 10, 1-9.
46. Dharmasri, P A, Levy, A D, & Blanpied, T A. “Differential nanoscale organization of excitatory synapses onto excitatory vs. inhibitory neurons.” PNAS, 2024, 121, 1-11.
47. Padfield D, O'Sullivan H, Pawar S. “rTPC and nls.multstart: A new pipeline to fit thermal performance curves in R.” Methods Ecol Evol. 2021, 12, 1138-1143.

What is claimed is:

1. A system for measuring single synaptic signals under varying experimental conditions, the system comprising:
a video recording microscope configured to view, and capture as a video frame, an imaged area in a sample holder disposed on a stage wherein the sample holder is configured to contain a sample of neuronal tissue combined with at least one fluorophore that emits a corresponding electromagnetic wavelength in a synapse during synaptic activity;
at least one processor; and
at least one memory including one or more sequences of instructions,
wherein the at least one memory and the one or more sequences of instructions configured to, with the at least one processor, cause the processor to perform at least the following:
control the video recording microscope to capture, at a plurality of different times, a first imaged area in the sample holder when the sample holder is disposed on the stage and holds the sample of neuronal tissue combined with the fluorophore;
determine at least one synaptic region of interest based on a group of a plurality of pixels in the first imaged area that record electromagnetic emissions from the at least one fluorophore at the plurality of different times;
record an ordered time series of emission intensity in each of the at least one synaptic region of interest at the plurality of times;
correct peak emission intensity values in the ordered time series for known fluorophore transients; and
store the ordered time series with corrected peak emission intensity values in a data structure with a standard format.

2. The system as recited in claim 1, wherein:
the at least one memory and the one or more sequences of instructions are further configured to cause the processor to perform the following steps before said step to correct peak emission intensity values in the time series for transmitter label transients.
remove a first set of outlier emission values from the ordered time series based on a rolling median value to produce a first baseline time series in each of the at least one synaptic region of interest;
record a rolling average time series of the first baseline time series in each of the at least one synaptic region of interest;
use the rolling average to determine a corrected normalized ordered time series of emission intensity and any potential fluorescence signal in each of the at least one synaptic region of interest;
record a new baseline ordered time series of emission intensity based on a rolling average excluding outliers and potential signals; and
use new baseline ordered time series of emission intensity to record a new normalized time ordered time series for each synapse for each fluorophore of the at least one fluorophore in each of the at least one synaptic region of interest.

3. The system as recited in claim 1, wherein:
the stage is moveable; and
the at least one memory and the one or more sequences of instructions are further configured to cause the processor to cause the moveable stage to move such that the video recording microscope views, and capture as a video frame, a second different imaged area in the sample holder.

4. The system as recited in claim 1, wherein:
the video recording microscope is a confocal microscope configured to bring into focus a selectable depth in the sample held in the sample holder on the stage; and
the at least one region of interest is a three dimensional region of interest comprising multiple depths in the sample.

5. The system as recited in claim 1, further comprising:
an electrical stimulation apparatus configured to apply an electrical voltage across a sample in the sample holder on the stage;
wherein the at least one memory and the one or more sequences of instructions are configured to, with the at least one processor, cause the processor to operate the electrical stimulation apparatus.

6. The system as recited in claim 1, further comprising:
a perfusion apparatus configured to introduce at least one fluid into the sample holder on the stage;
wherein the at least one memory and the one or more sequences of instructions configured to, with the at least one processor, cause the processor to operate the perfusion apparatus.

7. The system as recited in claim 6, wherein the fluid comprises a solution of calcium ions.

8. The system as recited in claim 1, wherein a first fluorophore of the at least one fluorophore is configured to emit a corresponding electromagnetic wavelength upon contact with glutamate.

9. The system as recited in claim 8, wherein the first fluorophore is iGluSnFR3.

10. The system as recited in claim 1, wherein a first fluorophore of the at least one fluorophore is bound to a protein in a wall of a vesicle that holds a neurotransmitter.

11. The system as recited in claim 10, wherein the first fluorophore is synaptophysin-mRuby.

12. The system as recited in claim 1, wherein a first fluorophore of the at least one fluorophore is iGluSnFR3 and a second fluorophore of the at least one fluorophore is synaptophysin-mRuby.

13. A method for operating a video recording microscope for measuring single synaptic signals under varying experimental conditions, the method comprising:
controlling a video recording microscope to capture, at a plurality of different times, a first imaged area in a sample holder as a video frame when the sample holder is disposed on a stage and holds a sample of neuronal tissue combined with at least one fluorophore that emits a corresponding electromagnetic wavelength in a synapse during synaptic activity;
determining at least one synaptic region of interest based on a group of a plurality of pixels in the first imaged area that record electromagnetic emissions from the at least one fluorophore at the plurality of different times;
recording an ordered time series of emission intensity in each of the at least one synaptic region of interest at the plurality of times;
correcting peak emission intensity values in the time series for known fluorophore transients; and
storing the ordered time series with corrected peak emission intensity values in a data structure with a standard format.

14. A non-transitory computer readable medium for operating a video recording microscope for measuring single synaptic signals under varying experimental conditions, the non-transitory computer readable medium carrying one or more sequences of instructions, wherein execution of the one or more sequences of instructions by one or more processors causes the one or more processors to perform the steps of:
controlling a video recording microscope to capture, at a plurality of different times, a first imaged area in a sample holder as a video frame when the sample holder is disposed on a stage and holds a sample of neuronal tissue combined with at least one fluorophore that emits a corresponding electromagnetic wavelength in a synapse during synaptic activity;
determining at least one synaptic region of interest based on a group of a plurality of pixels in the first imaged area that record electromagnetic emissions from the at least one fluorophore at the plurality of different times;
recording an ordered time series of emission intensity in each of the at least one synaptic region of interest at the plurality of times;
correcting peak emission intensity values in the time series for known fluorophore transients; and
storing the ordered time series with corrected peak emission intensity values in a data structure with a standard format.
